(19) United States
(12) Patent Application Publication
(10) Pub. No.: US 2026/0248608 A1
(43) Pub. Date: Aug. 27, 2026

(54) PROSTHETIC VALVE DOCKING DEVICE HAVING A GUARD MEMBER

(71) Applicant: Edwards Lifesciences Corporation, Irvine, CA (US)

(72) Inventors: Jocelyn Chau, Buena Park, CA (US); Kevin Gantz, Laguna Beach, CA (US); Bethany Jo Hall, Newport Beach, CA (US); Tri D. Tran, Fountain Valley, CA (US); Darshin S. Patel, Lake Forest, CA (US); Sean Chow, Irvine, CA (US); Paolo Mario Tartara, Newport Beach, CA (US); Tram Ngoc Nguyen, Santa Ana, CA (US); Kurt Kelly Reed, Costa Mesa, CA (US); Gianfranco Melino Pellegrini, Trabuco Canyon, CA (US)

(21) Appl. No.: 19/644,500

(22) Filed: Apr. 10, 2026

Related U.S. Application Data
(63) Continuation of application No. PCT/US2024/052205, filed on Oct. 21, 2024.
(60) Provisional application No. 63/592,503, filed on Oct. 23, 2023.

(51) Int. Cl. A61F 2/24

(52) U.S. Cl. CPC A61F 2/2409; A61F 2230/0091; A61F 2250/0063

(57) ABSTRACT

A docking device for securing a prosthetic valve at a native valve is provided. The docking device can include a coil and a guard member. The coil includes a plurality of helical turns when in a deployed orientation. The guard member is attached to the coil by being coupled to at least a portion of a helical turn thereof. The guard member includes a scaffold with a spine and a plurality of arms extending from the spine. The plurality of arms is coupled to a flap. The guard member is movable between a radially compressed state in a delivery orientation and a radially expanded state in the deployed orientation.

### CROSS-REFERENCE TO RELATED APPLICATIONS

This application is a continuation of PCT Patent Application No. PCT/US2024/52205 filed on October 21, 2024, which claims the benefit of U.S. Provisional Application No 63/592,503, filed October 23, 2023, each of these applications being incorporated herein by reference in its entirety.

### FIELD

The present disclosure concerns examples of guard members and coils for a docking device that is configured to improve placement and retention of the docking device and a prosthetic valve that fits into the docking device at a native heart valve, as well as methods of assembling such devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic valves can be used to treat cardiac valvular disorders. Native heart valves (for example, the aortic, pulmonary, tricuspid and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (for example, a native aortic valve). As such, the THV may not be suitable for implantation at another native valve (for example, a native mitral valve) and/or in a patient with a larger native valve. Additionally, or alternatively, the native tissue at the implantation site may not provide sufficient structure for the THV to be secured in place relative to the native tissue. Accordingly, improvements to THVs and the associated transcatheter delivery apparatus are desirable.

### SUMMARY

The present disclosure relates to methods and devices for treating valvular regurgitation and/or other valve issues. Specifically, the present disclosure is directed to a docking device configured to receive a prosthetic valve and the methods of assembling the docking device and implanting the docking device. The disclosed prosthetic heart valves, delivery apparatus, and methods can, for example, provide improved docking device stability in the transient stage of implantation and a lower delivery profile. As such, the devices and methods disclosed herein can, among other things, overcome one or more of the deficiencies of typical prosthetic heart valves and their delivery apparatus.

In some examples, a docking device is provided for securing a prosthetic valve at a native valve. The docking device includes a coil and guard member. The coil can include a plurality of helical turns when in a deployed orientation. The guard member can be attached to the coil by being coupled to at least a portion of a helical turn thereof. The guard member can be configured to include a scaffold with a spine and a plurality of arms extending from the spine. The plurality of arms is coupled to a flap to form the guard member. The guard member is movable between a radially compressed state in a delivery orientation and a radially expanded state in the deployed orientation. Accordingly, the scaffold can include a shape memory material set in the deployed orientation, which is compressible into the delivery orientation.

In some examples, a method for making the docking device of one of the examples is provided. The method includes obtaining the scaffold with the spine and plurality of arms connected to and extending radially outwardly from the spine. The method can include coupling the scaffold to the flap to form the guard member. The method can include obtaining the coil, and coupling the guard member to the coil.

In some examples, a method of configuring a docking device for delivery to a native valve is provided. The method can include providing the docking device of one of the examples, and compressing the guard member by compressing the arms to fold the flap into the delivery orientation. The method can include inserting the guard member in the delivery orientation into a dock sleeve of a dock delivery system.

In some examples, a method of implanting a docking device into a native valve is provided. The method can include providing the docking device of one of the examples, and delivering the docking device to a native valve while the docking device is in a delivery orientation. The method can include deploying the coil of the docking device at an annulus of the native valve, and then deploying the guard member into the deployed orientation at a position at the native valve so that the guard member overlays or presses against the native valve and/or native heart chamber associated with the native valve.

In some examples, a method of implanting a prosthetic valve is provided. The method can include providing the docking device of one of the examples and delivering the docking device to a native valve. The method can include deploying the docking device at an annulus of the native valve so that the guard member expands into the deployed orientation at a position at the native valve so that the guard member overlays or presses against the native valve and/or native heart chamber associated with the native valve. The method can include deploying a prosthetic valve within the docking device. During the delivery, the coil remains in a substantially straight delivery orientation when delivering the docking device and moves to a helical configuration after the docking device is in the deployed orientation. The guard member remains in a folded delivery orientation when delivering the docking device and moves to an unfolded deployed orientation after the docking device is deployed.

In some examples, a coil for a docking device for securing a prosthetic valve, the coil comprises a longitudinal axis extending through a lumen of the coil from an inflow side to an outflow side; a first coil region defining a first lumen diameter and configured to be disposed on the inflow side of a native annulus and to stabilize the coil relative to the native annulus; and a second coil region extending from a distal end of the first coil region and comprising one or more helical turns each defining a second lumen diameter and configured to be disposed on an outflow side of a native annulus and to receive a prosthetic valve, wherein a proximal end portion of the first coil region is lifted relative to a plane defined by the first coil region and normal to the longitudinal axis and wherein a proximal end of the first coil region is above the plane defined by the first coil region by less than 12 mm.

In some examples, the coil further comprises a leading coil extending from the distal end of the second coil region and extending radially outward from the second diameter. In some examples, the proximal end portion of the first coil region is lifted at an angle relative to a plane defined by the first coil region and normal to the longitudinal axis. In some examples, the angle is within a range of 10 to 50 degrees. In some examples, an attachment portion comprising one or more eyeholes is disposed at the proximal end portion of the first coil region.

In some examples, the first lumen diameter and the second lumen diameter are substantially equal. In some examples, the first lumen diameter is in a range of 10 percent to 30 percent greater than the second lumen diameter. In some examples, the first lumen diameter is in a range of 25 mm to 30 mm and the second lumen diameter is in a range of 20 mm to 25 mm. In some examples, the first coil region comprises a single stabilization turn.

In some examples, a docking device comprises a guard member coupled at least partially to an outflow side of the stabilization turn wherein the guard member is movable between a radially compressed state and a radially expanded state. In some examples, the guard member comprises a scaffold and the scaffold comprises a spine, a plurality of arms, and one or more terminal lobes. In some examples, the spine of the scaffolding further comprises a kickout portion configured to wrap around the stabilization turn. In some examples, the guard member comprises a lateral terminal lobe and a medial terminal lobe, and wherein the kickout portion is located proximal to the medial terminal lobe.

In some examples, the scaffold further comprises one or more retention elements. In some examples, the retention elements comprise tines, wherein the tines are attached to the arms at a base portion and wherein the tines taper to a point at a tip portion.

In some examples, the coil comprises: a longitudinal axis extending through a lumen of the coil from an inflow side to an outflow side; a first coil region configured to be disposed on an inflow side of a native annulus and to stabilize the coil relative to the native annulus; and a second coil region extending from a distal end of the first coil region and comprising one or more helical turns configured to be disposed on an outflow side of a native annulus and to receive a prosthetic valve wherein the coil omits a raised stabilization portion.

In some examples, the coil comprises a leading coil extending from the distal end of the second coil region and extending radially outward from a diameter of the second coil region.

In some examples, a proximal end portion of the first coil region is lifted at an angle relative to a plane defined by the first coil region and normal to the longitudinal axis. In some examples, the angle is within a range of 10 to 30 degrees. In some examples, the proximal end of the first coil region is above the plane defined by the first coil region by less than 12 mm.

In some examples, the first coil region defines a first lumen diameter and the second coil region defines a second lumen diameter. In some examples, the first lumen diameter and the second lumen diameter are substantially equal. In some examples, the first lumen diameter is in a range of 10 percent to 30 percent greater than the second lumen diameter. In some examples, the first lumen diameter is in a range of 25 mm to 30 mm and the second lumen diameter is in a range of 20 mm to 25 mm.

In some examples, a docking device comprises the coil of any example herein, and further comprising a guard member coupled at least partially to an outflow side of the stabilization coil wherein the guard member is movable between a radially compressed state and a radially expanded state. In some examples, the guard member comprises a scaffold and the scaffold comprises a spine, a plurality of arms, and one or more terminal lobes. In some examples, the guard member is coupled to the outflow side of the stabilization coil and extends circumferentially between 180 and 330 degrees on the outflow side of the stabilization turn. In some examples, the spine of the scaffolding further comprises a kickout portion configured to wrap around the stabilization turn. In some examples, a portion of the guard member wraps around the stabilization turn and extends circumferentially between 30 and 135 degrees on an inflow side of the stabilization turn.

In some examples, the scaffold further comprises one or more retention elements. In some examples, the retention elements comprise tines, wherein the tines are coupled to the arms at a base portion and wherein the tines taper to a point at a tip portion.

In some examples, a docking device for securing a prosthetic implant at a native valve, the docking device comprises: a coil defining a longitudinal axis extending through a lumen of the coil from an inflow side to an outflow side and comprising a plurality of helical turns when deployed at the native valve, wherein at least one of the helical turns comprises a first coil region configured to be disposed on the inflow side of a native annulus and to stabilize the coil relative to the native annulus wherein a proximal end portion of the first coil region is lifted relative to a plane defined by the first coil region and normal to the longitudinal axis and wherein a proximal end of the first coil region is above the plane defined by the first coil region by less than 12 mm and at least one of the helical turns comprises a second coil region extending from a distal end of the first coil region and configured to be disposed on the outflow side of a native annulus and to receive a prosthetic valve; and a guard member coupled at least partially to the outflow side of the first coil region wherein the guard member is movable between a radially compressed state and a radially expanded state.

In some examples, a portion of the guard member is coupled at least 225 degrees around a circumference of the first coil region. In some examples, a portion of the guard member is coupled at least 270 degrees around a circumference of the first coil region. In some examples, a portion of the guard member is coupled to the outflow side of the first coil region at least 270 degrees around a circumference of the first coil region and a portion of the guard member is disposed on the inflow side of the first coil region at least 15 degrees around a circumference of the of the first coil region.

In some examples, the guard member comprises a scaffold and the scaffold comprises a spine, a plurality of arms, and one or more terminal lobes. In some examples, the spine of the scaffold further comprises a kickout portion configured to wrap around the stabilization turn. In some examples, the scaffold further comprises one or more retention elements. In some examples, the retention elements comprise tines, wherein the tines are coupled to the arms at a base portion and wherein the tines taper to a point at a tip portion.

In some examples, a method comprises: delivering a docking device of any one of to a native valve; deploying the docking device at an annulus of the native valve; and deploying a prosthetic valve within the docking device, wherein the coil remains in a substantially straight configuration when delivering the docking device and moves to a helical configuration after the docking device is deployed.

In some examples, a coil for a docking device for securing a prosthetic valve comprises: a core comprising a plurality of helical turns and defining a longitudinal axis extending through a lumen of the plurality of helical turns from an inflow side to an outflow side when deployed at a native valve, wherein at least one of the helical turns comprises a first region configured to be disposed on the inflow side of a native annulus and to stabilize the coil relative to the native annulus and at least one of the helical turns comprises a second region extending from a distal end of the first region and configured to be disposed on the outflow side of a native annulus and to receive a prosthetic valve; and a cover encompassing at least a portion of the core and comprising a first outer diameter and a second outer diameter which is larger than the first outer diameter, wherein the second region comprises the second outer dim.

In some examples, at least a portion of the first region is covered by a cover of the second diameter. In some examples, the cover comprises a first cover with a first outer diameter and as second cover with a second outer diameter, wherein the first cover and the second cover are two separate pieces. In some examples the coil comprises a transition region, in which the first cover is flared radially outward such that it overlaps with the second cover in an axial direction. In some examples, a coupling member is wrapped around the first cover. In some examples, the coupling member comprises suture.

In some examples, a guard member, for a docking device for securing a prosthetic implant at a native valve, the guard member comprising a scaffold with a spine and a plurality of arms extending from the spine; and one or more retention elements coupled to one of the arms of the plurality of arms; wherein the guard member is configured to be attached to a coil by being coupled to at least a portion of a helical turn thereof, wherein the guard member is movable between a radially compressed state in a delivery orientation and a radially expanded state in a deployed orientation.

In some examples, the guard member comprises a flap wherein the plurality of arms are coupled to the flap and at least a portion of the scaffold is encompassed by the flap, and wherein the one or more retention elements extend though the flap. In some examples, the retention elements are tines configured to engage heart tissue to help ensure device stability. In some examples, the tines comprise a base portion which is coupled to the arm and has a first width, and a tip portion comprising a second width, wherein the second width is smaller than the first width. In some examples, the tines comprise a tip portion that is a point.

In some examples, when the guard member is in the radially expanded state in the deployed orientation, the plurality of arms and flap extend radially outward away from the coil and circumferentially along a portion of the coil. In some examples, when the guard member is in the radially expanded state, the retention elements extend from the arms in a clockwise direction. In some examples, the tines extend out of a plane defined by the scaffold. In some examples, an angle in a range of 0 to 90 degrees is formed between the tines and the plane defined by the scaffold. In some examples, an angle in a range of 0 to 45 degrees is formed between the tines and the plane defined by the scaffold. In some examples, an angle in a range of 90 to 180 degrees is formed between the tines and the plane defined by the scaffold.

The methods described herein can be performed on a living animal or on a simulation, such as on a cadaver, cadaver heart, anthropomorphic ghost, simulator (for example, with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

FIG. 1A is a cutaway view of the human heart in a diastolic phase.

FIG. 1B is a cutaway view of the human heart in a systolic phase.

FIG. 2A schematically illustrates a first stage in an exemplary mitral valve replacement procedure where a guide catheter and a guidewire are inserted into a vasculature of a patient and navigated through the vasculature and into a heart of the patient, towards a native mitral valve of the heart.

FIG. 2B schematically illustrates a second stage in the exemplary mitral valve replacement procedure where a docking device delivery apparatus extending through the guide catheter is used to deploy a docking device at the native mitral valve.

FIG. 3A schematically illustrates a third stage in the exemplary mitral valve replacement procedure where the docking device of FIG. 2B is fully implanted at the native mitral valve of the patient and the docking device delivery apparatus has been removed from the patient.

FIG. 3B schematically illustrates a fourth stage in the exemplary mitral valve replacement procedure where a prosthetic heart valve delivery apparatus extending through the guide catheter is used to deploy a prosthetic heart valve within the implanted docking device at the native mitral valve.

FIG. 4A schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4B schematically illustrates a sixth stage in the exemplary mitral valve replacement procedure where the guide catheter and the guidewire have been removed from the patient.

FIG. 5A includes a top view that illustrates an example of a coil of a docking device.

FIG. 5B includes a top view that illustrates an example of a scaffold of a guard member of a docking device.

FIG. 5C includes a top view that illustrates an example of a flap of a guard member of a docking device.

FIG. 6A includes a perspective view that illustrates an example of an assembled docking device having the coil and guard member.

FIG. 6B includes a top view of the docking device of FIG. 6A.

FIG. 6C includes a cross-sectional profile as marked with the arrows at 6C in FIG. 6A.

FIG. 6D includes a cross-sectional profile as marked with the arrows at 6D in FIG. 6A.

FIG. 7A includes a top view of a guard member in a radially expanded state in a deployed orientation.

FIG. 7B includes a top view of the guard member of FIG. 7A in a substantially straight configuration in a transition towards a delivery orientation.

FIG. 7C includes a top view of the guard member of FIG. 7B being radially compressed in the delivery orientation.

FIG. 7D includes a top view of the guard member of FIG. 7C in the delivery orientation in a delivery sleeve of a delivery device.

FIG. 8A includes a top view of an example of a docking device having a guard member with arm sleeves.

FIG. 8B includes a top view of an example of a docking device having a guard member with arms coupled with a flap.

FIG. 9A includes a top view of an example of a scaffold of a guard member.

FIG. 9B includes a top view of another example of a scaffold with fewer arms.

FIG. 9C includes a top view of another example of a scaffold with only arms.

FIG. 9D includes a top view of another example of a scaffold with a serpentine arm.

FIG. 9E includes a top view of another example of a scaffold with arms without heads.

FIG. 9F includes a top view of another example of a scaffold with arms formed as lobes.

FIG. 10A includes a top view of an example of a scaffold having markers.

FIG. 10B includes a cross-sectional view of an example of a coupling of a scaffold to a coil of a docking device.

FIG. 10C includes a cross-sectional view of an example of a coupling of a scaffold to a coil of a docking device.

FIG. 11A includes a top view of an example of a scaffold of a guard member having a peripheral lip with an edge protector.

FIG. 11B includes a perspective view of an example of a docking device with a coil having a reduced thickness at the guard member.

FIG. 12. shows the docking device being implanted into the mitral valve so that the guard member is on the left atrium side, with the proximal coil region extending into the left atrium, wherein as shown, the arms are extended in the deployed orientation so that the panels provide cover over the mitral valve anatomy, which can block a paravalvular leak (PVL).

FIG. 13 shows the docking device being implanted into the mitral valve so that the guard member is on the left atrium side, with the proximal coil region extending into the left atrium, wherein compared to FIG. 12, the mitral anatomy in FIG. 13 is smaller.

FIG. 14A shows a top view of an example of a docking device with a guard member, which is shown without the valve device.

FIG. 14B shows a top view of the example of the docking device with a guard member of FIG. 14A, which is shown with the valve device.

FIG. 15 shows a view of a scaffold where each of the arms taper from a wider base to a narrower end region adjacent to the head.

FIGS. 16 and 16A show examples of a scaffold with two terminal lobes. In FIG. 16, the scaffold is flat and planar. In FIG. 16A, one of the lobes is bent out of plane to form a scooped tip (e.g., ski tip).

FIG. 16B shows a perspective view and a side view of the example of FIG. 16A.

FIGS. 17A-17B depict a coil for a docking device with an attachment portion disposed at a proximal end portion of the stabilization turn.

FIG. 18 depicts a coil with a stabilization turn which has a larger diameter than the central region.

FIGS. 19A-19B depict a docking device comprising the coil of FIGS. 17A-17B with a guard member attached.

FIGS. 20A-20B depicts a heart valve disposed within a central lumen of the docking device of FIGS. 19A-19B.

FIG. 21A depicts a scaffold with a plurality of arms, two terminal lobes, and a kickout portion.

FIG. 21B-21E depicts a scaffold with plurality of arms, two terminal lobes, and retention elements.

FIG. 22 depicts a coil for a docking device with covers of different outer diameters which cover different segments of the coil.

FIG. 23 depicts a coil for a docking device with covers of different outer diameters which cover different segments of the coil, according to another example.

FIG. 24A-24B depict segments of a coil, such as the coils depicted in FIGS. 22-23 with covers of different diameters.

FIGS. 25A-25B depict a docking device comprising the coil of FIG. 22 with a guard member attached.

FIG. 26 depicts an example of a portion of a cover for a coil, including a transition region for covers of different diameters.

FIG. 27 depicts an example of a transition region for segments of a coil cover with different diameters.

FIG. 28 depicts an example of a transition region for segments of a coil cover with different diameters, according to another example.

### DETAILED DESCRIPTION

### General Considerations

The disclosed examples can be adapted to deliver and implant prosthetic devices in any of the native annuluses of the heart (for example, the pulmonary, mitral, and tricuspid annuluses), and can be used with any of various delivery approaches (for example, retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (for example, mechanically or chemically) coupled or linked and does not exclude the presence of intermediate elements between the coupled or associated items absent specific contrary language. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part, and the object remains the same. In the context of the present application, the terms “lower” and “upper” are used interchangeably with the terms “inflow” and “outflow”, respectively. Thus, for example, typically the lower end of a valve or docking station as depicted in the figures is its inflow end and the upper end of the valve or docking station is its outflow end unless explicitly described otherwise.

The terms “longitudinal” and “axial” refer to an axis extending in the upstream and downstream directions, or in the proximal and distal directions, unless otherwise expressly defined.

Although there are alternatives for various components, features, parameters, operating conditions, etc., set forth herein, that does not mean that those alternatives are necessarily equivalent and/or perform equally well. Nor does it mean that the alternatives are listed in a preferred order unless stated otherwise.

As used herein, the terms “integrally formed” and “unitary construction” refer to a construction that does not require any sutures, fasteners, or other securing means to attach two portions of the construction together.

As used herein, the term “about” can refer to a number that is within plus or minus 5% of the value indicated.

### Exemplary Transcatheter Heart Valve Replacement Procedure

Described herein are various systems, apparatuses, methods, or the like, that can be used in or with delivery apparatuses to deliver a prosthetic implant (for example, a prosthetic valve, a docking device, etc.) into a patient body.

In certain examples, a delivery apparatus can be configured to deliver and implant a docking device at an implantation site, such as a native valve annulus. The docking device can be configured to more securely hold an expandable prosthetic valve implanted within the docking device, at the native valve annulus. For example, a docking device can provide or form a more circular and/or stable anchoring site, landing zone, or implantation zone at the implant site, in which a prosthetic valve can be expanded or otherwise implanted. By providing such anchoring or docking devices, replacement prosthetic valves can be more securely implanted and held at various valve annuluses, including at the mitral annulus which does not have a naturally circular cross-section.

In some examples, the docking device can be arranged within an outer shaft of the delivery apparatus. A sleeve shaft can cover or surround the docking device within the delivery apparatus and during delivery to a target implantation site. A pusher shaft can be disposed within the outer shaft, proximal to the docking device, and configured to push the docking device out of the outer shaft to position the docking device at the target implantation site. The sleeve shaft can also surround the pusher shaft within the outer shaft of the delivery apparatus. After positioning the docking device at the target implantation site, the sleeve shaft can be removed from the docking device and retracted back into the outer shaft of the delivery apparatus.

Fluid (for example, a flush fluid, such as heparinized saline or the like) can be provided to a pusher shaft lumen defined within an interior of the pusher shaft, a delivery shaft lumen defined between the sleeve shaft and the outer shaft of the delivery apparatus, and a sleeve shaft lumen defined between the pusher shaft and the sleeve shaft. By providing a consistent flow of fluid through these lumens of the delivery apparatus, stagnation of blood within the delivery apparatus can be reduced or avoided, thereby reducing a risk of thrombus formation.

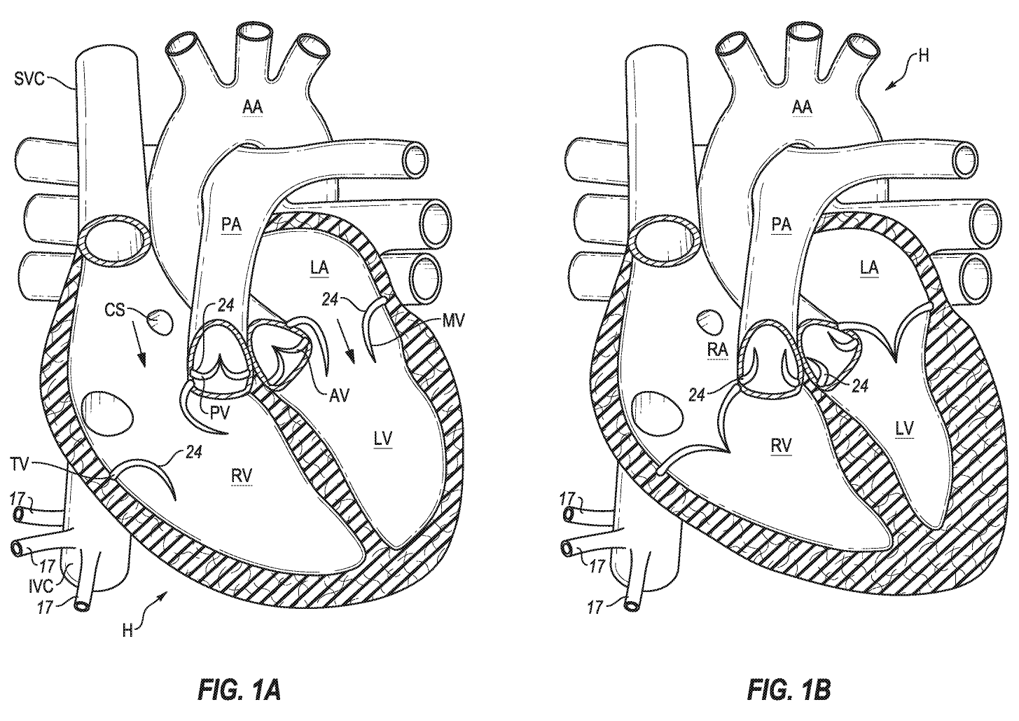

FIGS. 1A and 1B are cutaway views of the human heart (H) in diastolic and systolic phases, respectively. The right ventricle (RV) and left ventricle (LV) are separated from the right atrium (RA) and left atrium (LA), respectively, by the tricuspid valve (TV) and the mitral valve (MV); i.e., the atrioventricular valves. Additionally, the aortic valve (AV) separates the left ventricle (LV) from the ascending aorta (AA) and the pulmonary valve (PV) separates the right ventricle from the pulmonary artery (PA). Each of these valves has flexible leaflets extending inward across the respective orifices that come together or “coapt” in the flowstream to form one-way, fluid-occluding surfaces. The docking stations of the present application are described, for illustration, primarily with respect to the inferior vena cava (IVC), superior vena cava (SVC), mitral valve MV, and aorta/aortic valve. A defective mitral valve can suffer from insufficiency and/or regurgitation.

The blood vessels, such as the aorta, inferior vena cava IVC, superior vena cava SVC, pulmonary artery PA, may be healthy or may be dilated, distorted, enlarged, have an aneurysm, or be otherwise impaired. Anatomical structures of the right atrium RA, right ventricle RV, left atrium LA, and left ventricle LV will be explained in greater detail. The devices described herein can be used in various areas whether explicitly described herein or not, e.g., in the inferior vena cava IVC and/or superior vena cava SVC, in the aorta (e.g., an enlarged aorta) as treatment for a defective mitral valve, in other areas of the heart or vasculature, in grafts, etc.

The right atrium RA receives deoxygenated blood from the venous system through the superior vena cava SVC and the inferior vena cava IVC, the former entering the right atrium from above, and the latter from below. The hepatic veins 17 carry blood from the liver to the inferior vena cava IVC. The coronary sinus (CS) is a collection of veins joined together to form a large vessel that collects deoxygenated blood from the heart muscle (myocardium), and delivers it to the right atrium RA. During the diastolic phase, or diastole, seen in FIG. 1A, the deoxygenated blood from the inferior vena cava IVC, superior vena cava SVC, and coronary sinus CS that has collected in the right atrium RA passes through the tricuspid valve TV and into the right ventricle RV as the right ventricle RV expands, while blood from the left atrium LA passes through the mitral valve MV into the left ventricle LV. In the systolic phase, or systole, seen in FIG. 1B, the right ventricle RV contracts to force the deoxygenated blood collected in the right ventricle RV through the pulmonary valve PV and pulmonary artery into the lungs, while the left ventricle LV contracts to force blood in the left ventricle through the mitral valve MV into the left atrium LA.

The devices described herein can be used to supplement the function of a defective mitral valve. During systole, the leaflets of a normally functioning mitral valve MV close to prevent the blood from regurgitating back into the left atrium LA. When the mitral valve MV does not operate normally, blood can backflow or regurgitate into the left atrium LA. Blood regurgitating backward into the left atrium LA increases the volume of blood in the atrium and the blood vessels that direct blood to the heart. This can cause the left atrium LA to enlarge and cause blood pressure to increase in the left atrium LA and blood vessels, which can cause damage to and/or swelling of the liver, kidneys, legs, other organs, etc. A transcatheter valve (THV) implanted in the mitral valve MV can inhibit blood from backflowing into the left atrium LA during the systolic phase.

The left atrium LA receives oxygenated blood from the left and right pulmonary veins, which then travels through the mitral valve to the left ventricle. During the diastolic phase, or diastole, seen in FIG. 1A, the oxygen rich blood that collects in the left atrium LA passes through the mitral valve MV and into the left ventricle LV as the left ventricle LV expands. In the systolic phase, or systole, seen in FIG. 1B, the left ventricle LV contracts to force the oxygen rich blood through the aortic valve AV and aorta into the body through the circulatory system. In certain examples, the devices described herein can be used to supplement or replace the function of a defective mitral valve MV.

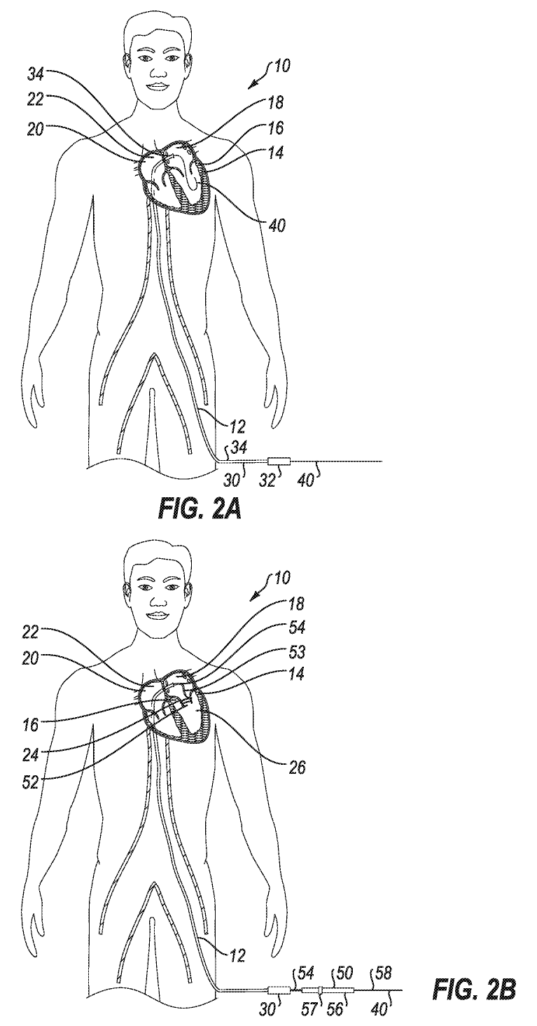

An exemplary transcatheter heart valve replacement procedure which utilizes a first delivery apparatus to deliver a docking device to a native valve annulus and then a second delivery apparatus to deliver a prosthetic transcatheter heart valve (for example, THV) inside the docking device is depicted in the schematic illustrations of FIGS. 2A-4B.

As introduced above, defective native heart valves may be replaced with THVs. However, in certain instances, such THVs may not be able to sufficiently secure themselves to the native tissue (for example, to the leaflets and/or annulus of the native heart valve) and may undesirably shift around relative to the native tissue, leading to paravalvular leakage (PVL), valve malfunction, and/or other issues. Thus, a docking device may be implanted first at the native valve annulus and then the THV can be implanted within the docking device to help anchor the THV to the native tissue and provide a seal between the native tissue and the THV.

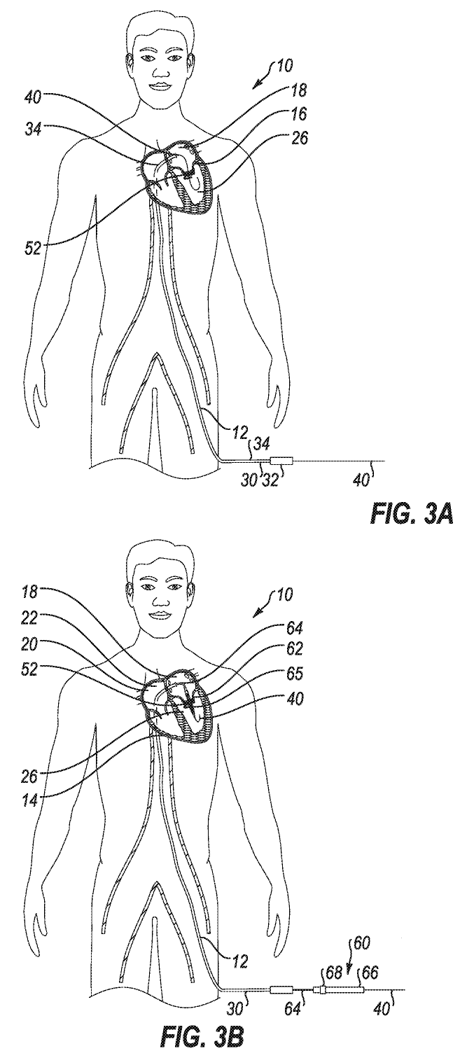

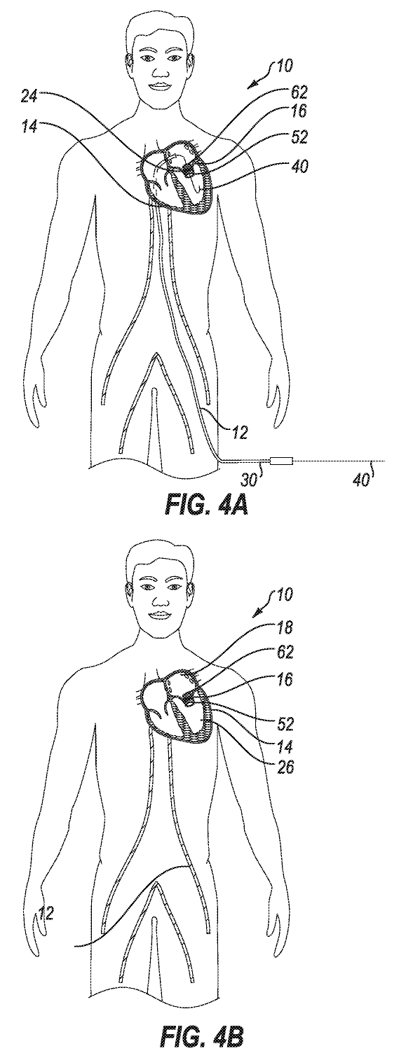

FIGS. 2A-4B depict an exemplary transcatheter heart valve replacement procedure (for example, a mitral valve replacement procedure) which utilizes a docking device 52 (e.g., with guard member as described herein) and a prosthetic heart valve 62, according to one example. During the procedure, a user can create a pathway to a patient’s native heart valve using a guide catheter 30 (FIG. 2A). The user can deliver and implant the docking device 52 at the patient’s native heart valve using a docking device delivery apparatus 50 (FIG. 2B) and then removes the docking device delivery apparatus 50 from the patient 10 after implanting the docking device 52 (FIG. 3A). The user can then implant the prosthetic heart valve 62 within the implanted docking device 52 using a prosthetic valve delivery apparatus 60 (FIG. 3B). Thereafter, the user can remove the prosthetic valve delivery apparatus 60 from the patient 10 (FIG. 4A), as well as the guide catheter 30 (FIG. 4B).

FIG. 2A depicts a first stage in a mitral valve replacement procedure, according to one example. As shown, the guide catheter 30 and a guidewire 40 can be inserted into a vasculature 12 of a patient 10 and navigated through the vasculature 12, into a heart 14 of the patient 10, and toward the native mitral valve 16 (e.g., through heart tissue wall between right atrium RA to left atrium LA as shown). Together, the guide catheter 30 and the guidewire 40 can provide a path for the docking device delivery apparatus 50 and the prosthetic valve delivery apparatus 60 to be navigated through and along, to the implantation site (for example, the native mitral valve 16 or native mitral valve annulus).

Initially, the user may first make an incision in the patient’s body to access the vasculature 12. For example, as illustrated in FIG. 1, the user may make an incision in the patient’s groin to access a femoral vein. Thus, in such examples, the vasculature 12 may include a femoral vein.

After making the incision to access the vasculature 12, the user may insert the guide catheter 30, the guidewire 40, and/or additional devices (such as an introducer device or transseptal puncture device) through the incision and into the vasculature 12. The guide catheter 30 (which can also be referred to as an “introducer device,” “introducer,” or “guide sheath”) can be configured to facilitate the percutaneous introduction of various implant delivery devices (for example, the docking device delivery apparatus 50 and the prosthetic valve delivery apparatus 60) into and through the vasculature 12 and may extend through the vasculature 12 and into the heart 14 but may stop short of the native mitral valve 16. The guide catheter 30 can comprise a handle 32 and a shaft 34 extending distally from the handle 32. The shaft 34 can extend through the vasculature 12 and into the heart 14 while the handle 32 can remain outside the body of the patient 10 and can be operated by the user in order to manipulate the shaft 34 (FIG. 2A).

The guidewire 40 can be configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the vasculature 12 and into a left atrium 18 of the heart 14 (and in some examples, through the native mitral valve 16 and into a left ventricle 26 of the heart 14) (FIG. 2A).

In some instances, a transseptal puncture device or catheter can be used to initially access the left atrium 18, prior to inserting the guidewire 40 and the guide catheter 30. For example, after making the incision to access the vasculature 12, the user may insert a transseptal puncture device through the incision and into the vasculature 12. The user may guide the transseptal puncture device through the vasculature 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the vasculature 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the vasculature 12 and advance the guide catheter 30 into the left atrium 18 over the guidewire 40 (FIG. 2A).

In some instances, an introducer device can be inserted through a lumen of the guide catheter 30 prior to inserting the guide catheter 30 into the vasculature 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2B depicts a second stage in the exemplary mitral valve replacement procedure where a docking device 52 can be implanted at the native mitral valve 16 of the heart 14 of the patient 10 using a docking device delivery apparatus 50 (which may also be referred to as an “implant catheter,” or a “docking device delivery device,” or simply “delivery apparatus”).

In general, the docking device delivery apparatus 50 can include a delivery shaft 54 (which may also be referred to as an “outer shaft”), a handle 56, and a pusher assembly 58 (which may also be referred to as a “pusher shaft”). The delivery shaft 54 can be configured to be advanced through the patient’s vasculature 12 and to the implantation site (for example, native mitral valve 16) by the user, and may be configured to retain the docking device 52 in a distal end portion 53 of the delivery shaft 54. In some examples, the distal end portion 53 of the delivery shaft 54 can retain the docking device 52 therein in a substantially straight delivery orientation.

The handle 56 of the docking device delivery apparatus 50 can be configured to be gripped and/or otherwise held by the user to advance the delivery shaft 54 through the patient’s vasculature 12. Specifically, the handle 56 can be coupled to a proximal end of the delivery shaft 54 and can be configured to remain accessible to the user (for example, outside the body of the patient 10) during the docking device implantation procedure. In this way, the user can advance the delivery shaft 54 through the patient’s vasculature 12 by exerting a force on (for example, pushing) the handle 56. In some examples, the delivery shaft 54 can be configured to carry the pusher assembly 58 and/or the docking device 52 with it as it advances through the patient’s vasculature 12. In this way, the docking device 52 and/or the pusher assembly 58 can advance through the patient’s vasculature 12 in lockstep with the delivery shaft 54 as the user grips the handle 56 and pushes the delivery shaft 54 deeper into the patient’s vasculature 12.

In some examples, the handle 56 can comprise one or more articulation members 57 that are configured to aid in navigating the delivery shaft 54 through the vasculature 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the vasculature 12 and/or within the heart 14.

The pusher assembly 58 can be configured to deploy and/or implant the docking device 52 at the implantation site (for example, the native mitral valve 16). For example, the pusher assembly 58 can be configured to be adjusted by the user to push the docking device 52 out of the distal end portion 53 of the delivery shaft 54. A pusher shaft of the pusher assembly 58 can extend through the delivery shaft 54 and can be disposed adjacent to the docking device 52 within the delivery shaft 54. In some examples, the docking device 52 can be releasably coupled to the pusher shaft of the pusher assembly 58 via a connection mechanism of the docking device delivery apparatus 50 such that the docking device 52 can be released after being deployed at the native mitral valve 16. Because the docking device 52 is retained by, held, and/or otherwise coupled to the pusher assembly 58, the docking device 52 can advance in lockstep with the pusher assembly 58 through and/or out of the delivery shaft 54.

In addition to the pusher shaft, in certain instances, the pusher assembly 58 can also include a sleeve shaft. The pusher shaft can be configured to advance the docking device 52 through the delivery shaft 54 and out of the distal end portion 53 of the delivery shaft 54, while the sleeve shaft, when included, can have a distal dock sleeve configured to cover the docking device 52 within the delivery shaft 54 and while pushing the docking device 52 out of the delivery shaft 54 and positioning the docking device 52 at the implantation site. In some examples, the pusher shaft can be covered, at least in part, by the sleeve shaft.

In some examples, the pusher assembly 58 can comprise a pusher handle that is coupled to the pusher shaft and that is configured to be gripped and pushed by the user to translate the pusher shaft axially relative to the delivery shaft 54 (for example, to push the pusher shaft into and/or out of the distal end portion 53 of the delivery shaft 54). The dock sleeve can be configured to be retracted and/or withdrawn from the docking device 52, after positioning the docking device 52 at the target implantation site. For example, the pusher assembly 58 can include a sleeve handle that is coupled to the sleeve shaft and is configured to be pulled by a user to retract (for example, axially move) the sleeve shaft relative to the pusher shaft, thereby retracting the dock sleeve.

The pusher assembly 58 can be removably coupled to the docking device 52, and as such can be configured to release, detach, decouple, and/or otherwise disconnect from the docking device 52 once the docking device 52 has been deployed at the target implantation site. As just one example, the pusher assembly 58 may be removably coupled to the docking device 52 via a thread, string, yarn, suture, or other suitable material that is tied or sutured to the docking device 52.

In some examples, the pusher assembly 58 can include a suture lock assembly (also referred to as a “suture lock”) that is configured to receive and/or hold the thread or other suitable material that is coupled to the docking device 52 via a suture. The thread or other suitable material that forms the suture can extend from the docking device 52, through the pusher assembly 58, to the suture lock assembly. The suture lock assembly can also be configured to cut the suture to release, detach, decouple, and/or otherwise disconnect the docking device 52 from the pusher assembly 58. For example, the suture lock assembly can comprise a cutting mechanism that is configured to be adjusted by the user to cut the suture.

Referring again to FIG. 2B, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the vasculature 12 along the guidewire 40 until the delivery shaft 54 reaches the left atrium 18, as illustrated in FIG. 2B. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the vasculature 12 and the heart 14, the user may adjust the one or more articulation members 57 of the handle 56 to navigate the various turns, corners, constrictions, and/or other obstacles in the vasculature 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2B that is configured to be positioned within a left ventricle 26 and/or on the ventricular side of the native mitral valve 16), the user may then deploy the remaining portion of the docking device 52 (for example, an atrial portion of the docking device 52 having the brim feature) from the delivery shaft 54 within the left atrium 18 by retracting the delivery shaft 54 away from the medial commissure of the native mitral valve 16. For example, the user can maintain the position of the pusher assembly 58 (for example, by exerting a holding and/or pushing force on the pusher shaft) while retracting the delivery shaft 54 proximally so that the delivery shaft 54 withdraws and/or otherwise retracts relative to the docking device 52 and the pusher assembly 58. In this way, the pusher assembly 58 can hold the docking device 52 in place while the user retracts the delivery shaft 54, thereby releasing the docking device 52 from the delivery shaft 54. In some examples, the user can also remove the dock sleeve from the docking device 52, for example, by retracting the sleeve shaft. The brim feature that is described in more detail below can help facilitate retention of the docking device in the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50 (for example, by cutting the suture tied to the docking device 52), the user may retract the docking device delivery apparatus 50 out of the vasculature 12 and away from the patient 10 so that the user can deliver and implant a prosthetic heart valve 62 within the implanted docking device 52 at the native mitral valve 16.

FIG. 3A depicts a third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2B). As such, the guidewire 40 can help to guide the prosthetic valve delivery apparatus 60 through the annulus of the native mitral valve 16 and at least partially into the left ventricle 26.

As illustrated in FIG. 3A, the docking device 52 can comprise a plurality of helical turns that wrap around the leaflets 24 of the native mitral valve 16 (within the left ventricle 26). The implanted docking device 52 can have a more cylindrical shape than the annulus of the native mitral valve 16, thereby providing a geometry that more closely matches the shape or profile of the PHV to be implanted. As a result, the docking device 52 with the brim feature can provide a tighter fit, and thus a better seal, between the prosthetic heart valve and the native mitral valve 16, as described further below.

FIG. 3B depicts a fourth stage in the mitral valve replacement procedure where the user is delivering and/or implanting a prosthetic heart valve 62 within the docking device 52 using a prosthetic valve delivery apparatus 60.

As shown in FIG. 3B, the prosthetic valve delivery apparatus 60 can comprise a delivery shaft 64 and a handle 66. The delivery shaft 64 can extend distally from the handle 66. The delivery shaft 64 can be configured to extend into the patient’s vasculature 12 to deliver, implant, expand, and/or otherwise deploy the prosthetic heart valve 62 within the docking device 52 at the native mitral valve 16. The handle 66 can be configured to be gripped and/or otherwise held by the user to advance the delivery shaft 64 through the patient’s vasculature 12.

In some examples, the handle 66 can comprise one or more articulation members 68 that are configured to aid in navigating the delivery shaft 64 through the vasculature 12 and the heart 14. Specifically, the articulation members 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the vasculature 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3B, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 2D, the prosthetic heart valve 62 can be mounted around the expansion mechanism 65 (for example, the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (for example, the delivery shaft 64) into the patient 10 through the guide catheter 30 and over the guidewire 40. The user can continue to advance the prosthetic valve delivery apparatus 60 along the guidewire 40 (for example, through the vasculature 12) until the distal end portion of the delivery shaft 64 reaches the native mitral valve 16, as illustrated in FIG. 2D. More specifically, the user can advance the delivery shaft 64 of the prosthetic valve delivery apparatus 60 by gripping and exerting a force on (for example, pushing) the handle 66. While advancing the delivery shaft 64 through the vasculature 12 and the heart 14, the user can adjust the one or more articulation members 68 of the handle 66 to navigate the various turns, corners, constrictions, and/or other obstacles in the vasculature 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 2D, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3B), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52. In some examples, the user can lock the prosthetic heart valve 62 in its fully expanded position (for example, with a locking mechanism) to prevent the prosthetic heart valve 62 from collapsing.

FIG. 4A shows a fifth stage in the mitral valve replacement procedure where the prosthetic heart valve 62 in its radially expanded configuration and implanted within the docking device 52 in the native mitral valve 16. As shown in FIG. 4A, the prosthetic heart valve 62 can be received and retained within the docking device 52.

As also shown in FIG. 4A, after the prosthetic heart valve 62 has been fully deployed and implanted within the docking device 52 at the native mitral valve 16, the prosthetic valve delivery apparatus 60 (including the delivery shaft 64) can be removed from the patient 10 such that only the guidewire 40 and the guide catheter 30 remain inside the patient 10.

FIG. 4B depicts a sixth stage in the mitral valve replacement procedure, where the guidewire 40 and the guide catheter 30 have been removed from the patient 10. The docking device 52 with the brim feature can be configured to provide a seal between the prosthetic heart valve 62 and the leaflets 24 of the native mitral valve 16 to reduce paravalvular leakage around the prosthetic heart valve 62. Specifically, the docking device 52 can initially constrict the leaflets 24 of the native mitral valve 16, where the brim feature sits on top on the left atrial side. The prosthetic heart valve 62 can then push the leaflets 24 against the docking device 52 as it radially expands within the docking device 52. Thus, the docking device 52 and the prosthetic heart valve 62 can be configured to sandwich the leaflets 24 of the native mitral valve 16 when the prosthetic heart valve 62 is expanded within the docking device 52. In this way, the docking device 52 can provide a seal between the leaflets 24 of the native mitral valve 16 and the prosthetic heart valve 62 to reduce paravalvular leakage around the prosthetic heart valve 62.

In some examples, one or more of the docking device delivery apparatus 50, the prosthetic valve delivery apparatus 60, and/or the guide catheter 30 can comprise one or more fluid ports that are configured to supply flushing fluid to the lumens thereof to prevent and/or reduce the likelihood of blood clot (for example, thrombus) formation. Example fluid ports that can be used to inject flushing fluid into a docking device delivery apparatus are described further below.

Although FIGS. 2A-4B specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient’s vasculature along the guidewire 40 until the prosthetic heart valve 62 is positioned or disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 2A-4B depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

Additional examples of the docking device delivery apparatus, including its variants, and methods of implanting a docking device and implanting a prosthetic valve within the docking device are described in International Publication Nos. WO 2020/247907 and WO 2022/087336, and U.S. Patent Publication Nos. US2018/0318079, US2018/0263764, and US2018/0177594, which are all incorporated by reference herein in their entireties.

### Exemplary Prosthetic Valves

Details regarding the prosthetic heart valves described herein and various valve components are described U.S. Patent No. 11,185,406, which is incorporated herein by reference. Additional example prosthetic valves are described in International Patent Application Publication No. WO 2018/222799, U.S. Patent No. 9,155,619, and U.S. Patent Publication No. 2018/0028310, all of which are incorporated herein by reference in their entireties.

In some examples, the prosthetic heart valve comprises a plastically expandable material, which can be metal alloys, polymers, or combinations thereof. Example metal alloys can comprise one or more of the following: nickel, cobalt, chromium, molybdenum, titanium, or other biocompatible metal. In some examples, the prosthetic heart valve can comprise stainless steel, cobalt-chromium, nickel-cobalt-chromium, a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

In some examples, the prosthetic heart valve can be a self-expandable prosthetic valve with a frame made from a self-expanding material, such as nickel-titanium alloy or Nitinol. When the prosthetic valve is a self-expanding valve, the balloon of the delivery apparatus can be replaced with a sheath or similar restraining device that retains the prosthetic valve in a radially compressed state for delivery through the body. When the prosthetic valve is at the implantation location, the prosthetic valve can be released from the sheath, and therefore allowed to expand to its functional size. It should be noted that any of the delivery apparatuses disclosed herein can be adapted for use with a self-expanding valve.

### Overview of Docking Devices

Docking devices according to examples of the disclosure can, for example, provide a stable anchoring site, landing zone, or implantation zone at the implant site in which prosthetic valves can be expanded or otherwise implanted. Many of the disclosed docking devices comprise a circular or cylindrically-shaped portion, which can (for example) allow a prosthetic heart valve comprising a circular or cylindrically-shaped valve frame to be expanded or otherwise implanted into native locations with naturally circular cross-sectional profiles and/or in native locations with naturally with non-circular cross sections. In addition to providing an anchoring site for the prosthetic valve, the docking devices can be sized and shaped to cinch or draw the native valve (for example, mitral, tricuspid, etc.) anatomy radially inwards. In this manner, one of the main causes of valve regurgitation (for example, functional mitral regurgitation), specifically enlargement of the heart (for example, enlargement of the left ventricle, etc.) and/or valve annulus, and consequent stretching out of the native valve (for example, mitral, etc.) annulus, can be at least partially offset or counteracted. Some examples of the docking devices further include features which, for example, are shaped and/or modified to better hold a position or shape of the docking device during and/or after expansion of a prosthetic valve therein. By providing such docking devices, replacement valves can be more securely implanted and held at various valve annuluses, including at the mitral valve annulus which does not have a naturally circular cross-section.

In some instances, a docking device can comprise a paravalvular leakage (PVL) guard (also referred to herein as “a guard member”). The PVL guard can, for example, help reduce regurgitation and/or promote tissue ingrowth between the native tissue and the docking device.

The PVL guard can, in some examples, be movable between a delivery orientation (or radially compressed state) and a deployed orientation (or radially expanded state). When the PVL guard is in the delivery orientation, the PVL guard can extend along and adjacent the coil. When the PVL guard is in the deployed orientation, the PVL guard can rotate about a central longitudinal axis of the coil and extend radially outwardly from the coil.

### Exemplary Docking Devices

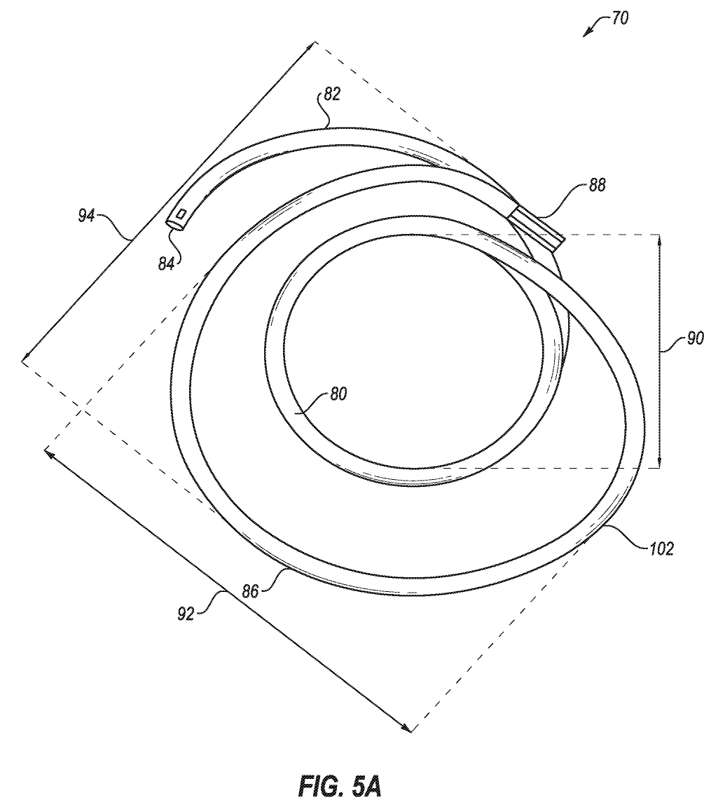

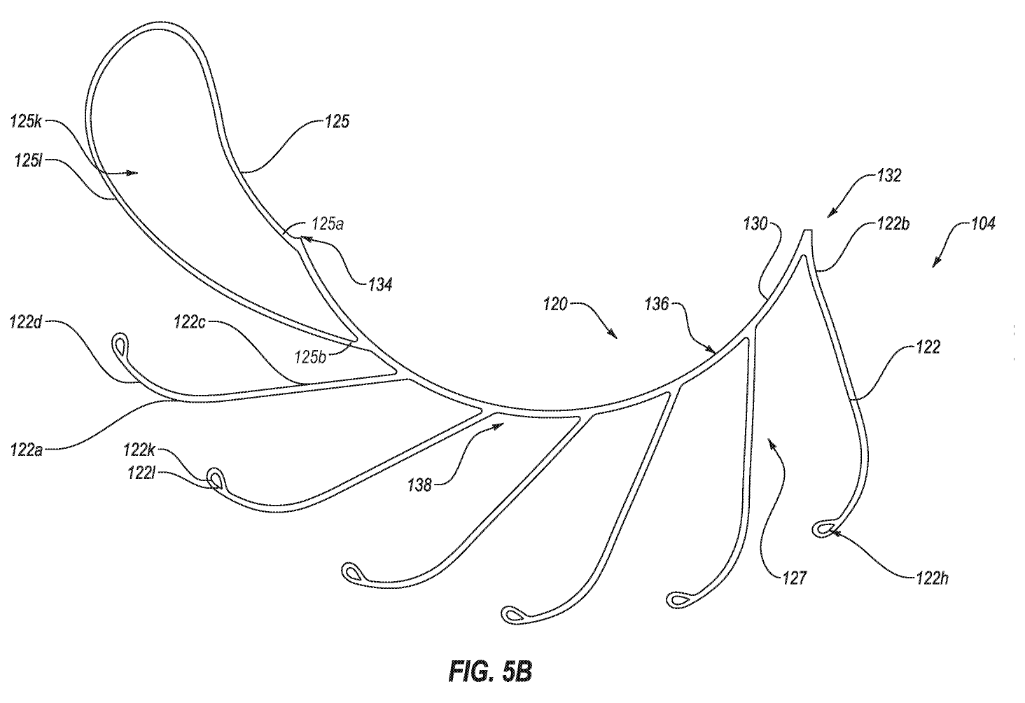

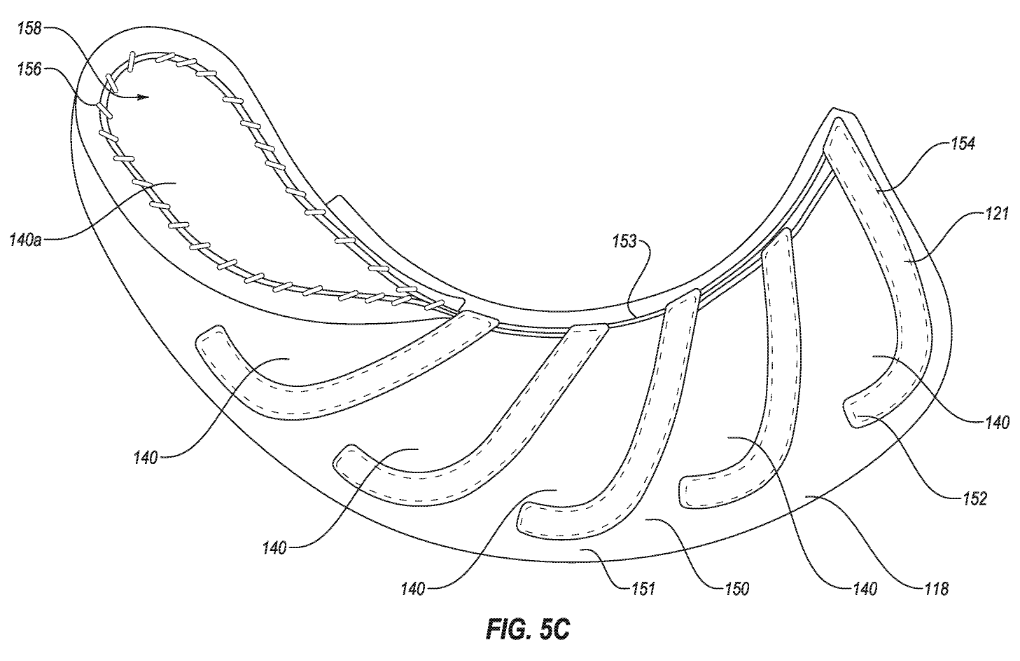

FIG. 5A illustrates a top view of an example of a hybrid docking device 70 or a core of a hybrid docking device 70 in accordance with various examples. The docking device 70 can be configured to fit at the mitral position but can be shaped and/or adapted similarly or differently in other examples for better accommodation at other native valve positions as well, such as at the tricuspid valve. Advantageously, the docking device geometries of the present disclosure provide for engagement with the native anatomy that can provide for increased stability and reduction of relative motion between the docking device, the prosthetic valve docked therein, and the native anatomy. Reduction of such relative motion can prevent material degradation of components of the docking device and/or the prosthetic valve docked therein and can prevent damage/trauma to the native tissues as well as preventing PVL.

The docking device 70 of many examples includes a central region 80 with a coil, coiled portion, or multiple coils (e.g., 1 coil, 2 coils, 3 coils, 4 coils, between 1–5 coils, or more). The coiled portion or coils of the central region 80 can be similarly sized and shaped or vary in size and/or shape. In some implementations, the central region 80 comprises three or approximately three full coil turns having substantially equal inner diameters. The central region 80 of the docking device 70 serves as the main landing region or holding region for holding the expandable prosthetic valve when the docking device 70 and the valve prosthesis are implanted into a patient’s body. In some examples, the docking device 70 has a central region 80 with more or less than three coil turns, depending for example, on the patient’s anatomy, the amount of vertical contact desired between the docking device 70 and the valve prosthesis (e.g., transcatheter heart valve or THV), and/or other factors. The coiled portion or coil(s) of the central region 80 can also be referred to as the “functional coils” or “functional turns” since the properties of these coils contribute the most to the amount of retention force generated between the valve prosthesis, the docking device 70, and the native mitral leaflets and/or other anatomical structures.

Various factors can contribute to the total retention force between the docking device 70 and the prosthetic valve held therein. For example, the shape of the valve to be implanted in the docking device, such as a valve with flared inflow and outflow which may naturally sit with the coil positioned in its narrower center. A main factor is the number of turns included in the functional coils, while other factors include, for example, an inner diameter of the functional coils, friction force (e.g., between the coils and the prosthetic valve), and the strength of the prosthetic valve and the radial force the valve applies on the coil. A docking device can have a variety of numbers of coils and/or turns. The number of functional turns can be in ranges from just over a half turn to 5 turns, or one full turn to 5 turns, or more. In one example with three full turns, an additional one-half turn is included in the ventricular portion of the docking device. In another example, there can be three full turns total in the docking device. In one example, in the atrial portion of the docking device, there can be one-half to three-fourths turn or one-half to three-fourths of a circle. While a range of turns is provided, as the number of turns in a docking device is decreased, the dimensions and/or materials of the coil and/or the wire that the coil is made from can also change to maintain a proper retention force. For example, the diameter of the wire can be larger and/or the diameter of the function coil turn(s) in a docking device with fewer coils. There can be a plurality of coils in the atrium and in the ventricle.

A size of the functional coils or coils of the central region 80 is generally selected based on the size of the desired THV to be implanted into the patient. Generally, the inner diameter 90 of the functional coils/turns (e.g., of the coils/turns of the central region 80 of the docking device 70) will be smaller than the outer diameter of the expandable heart valve, so that when the prosthetic valve is expanded in the docking device, additional radial tension or retention force will act between the docking device and the prosthetic valve to hold the prosthetic valve in place. The retention force needed for adequate implantation of a prosthetic valve varies based on the size of the prosthetic valve and on the ability of the assembly to handle mitral pressures of approximately 180 mm Hg. For example, based on hemodynamic data using a prosthetic valve with a 29 mm expanded outer diameter, a retention force of at least 15.8 N can be needed between the docking device and the prosthetic valve in order to securely hold the prosthetic valve in the docking device and to resist or prevent valve regurgitation or leakage. However, under this example, to meet this 15.8 N retention force requirement with statistical reliability, a target average retention force should be substantially greater, for example, approximately 30 N.

In many examples, the retention force between the docking device and the valve prosthesis reduces dramatically when a difference between the outer diameter of the prosthetic valve in its expanded state and the inner diameter of the functional coils is less than about 5 mm, since the reduced size differential can be too small to create sufficient retention force between the components. For example, when, in one example, a prosthetic valve with a 29 mm expanded outer diameter was expanded in a set of coils with a 24 mm inner diameter, the retention force observed was about 30 N, but when the same prosthetic valve was expanded in a set of coils with a 25 mm inner diameter (e.g., only 1 mm larger), the retention force observed dropped significantly to only 20 N. Therefore, in some examples, in order to create a sufficient retention force between the docking device and a 29 mm prosthetic valve, the inner diameter of the functional coils should be 24 mm or less. Often, the inner diameter of the functional coils (e.g., central region 80 of the docking device 70) should be selected to be at least about 5 mm less than the prosthetic valve that is selected for implantation, though other features and/or characteristics (e.g., friction enhancing features, material characteristics, etc.) can be used to provide better retention if other sizes or size ranges are used, as various factors can affect retention force.

However, diameter of the functional coils should be selected based on consideration and balancing of several factors to obtain optimal results. For example, the native anatomy between the mitral annulus at the mitral plane and the papillary muscle heads forms a generally trapezoidal shape, and the tissue of the mitral leaflets is thicker near the mitral plane and thins the further below the mitral plane. Smaller diameters of the central region 80 may encourage the docking device 70 to install further below the mitral plane than desirable (a similar effect can be observed at the tricuspid valve as well). When docking occurs at a location where the mitral leaflets are thinner, this may result in a suboptimal anchoring position for the prosthetic valve. Accordingly, size, diameters, and other features that help hold the prosthetic valve higher on the leaflets can be beneficial. In addition, a size of the inner diameter of the functional coils or central region 80 can also be selected to draw the native anatomy closer together, in order to at least partially offset or counteract valve regurgitation that is caused by stretching out of the native valve annulus as a result of, for example, left ventricular enlargement.

It is noted that the desired retention forces discussed above are applicable to examples for mitral valve replacements. Therefore, other examples of the docking device that are used for replacement of other valves can have different size relationships based on the desired retention forces for valve replacement at those respective positions. In addition, the size differentials can also vary, for example, based on the materials used for the valve and/or the docking device, whether there are any other features to prevent expansion of the functional coils or to enhance friction/locking, and/or based on various other factors.

In examples where the docking device 70 is used at the mitral position, the docking device can first be advanced and delivered to the native mitral valve annulus, and then set at a desired position, prior to implantation of the prosthetic heart valve. In some examples, the docking device 70 is flexible and/or made of a shape memory material, so that the coils of the docking device 70 can be straightened for delivery via a transcatheter approach as well. In some examples, the coil is made of another biocompatible material, such as stainless steel. Some of the same catheters and other delivery tools can be used for both delivery of the docking device 70 and the prosthetic valve, without having to perform separate preparatory steps, simplifying the implantation procedure for the end user.

Since the functional coils/turns or coils/turns of the central region 80 of the docking device 70 are kept relatively small in diameter (e.g., the central region 80 in one example can have an inner diameter of between approximately 21-24 mm (e.g., ± 2 mm) or another diameter smaller than the prosthetic valve and/or the native annulus) in order to increase retention force with the prosthetic valve, it might be difficult to advance the docking device 70 around the existing leaflets and/or chordae tendineae to a desired position relative to the native mitral annulus. This is especially true, if the entire docking device 70 is made to have the same small diameter as the central region 80. Therefore, the docking device 70 can have a distal or lower region 82 that comprises and/or consists of a leading coil/turn (sometimes referred to as an encircling turn or a leading ventricular coil/turn) of the docking device 70, which has a lower diameter that is greater than the diameter of the functional coils/turns or of the coils/turns of central region 80.

Features of the native anatomy, especially in the right and left ventricles, have variable dimensions. For example, native mitral anatomy can have an approximately 25 mm to 65 mm greatest width on a long axis. The diameter or width of the encircling turn or leading coil/turn (e.g., ventricular coil/turn) of the lower region 82 can be selected to be larger to more easily navigate a distal or leading tip 84 of the docking device 70 around and encircle the features of the native anatomy (e.g., leaflets and/or chordae tendineae).

Various sizes and shapes are possible, for example, in one example, the diameter could be any size from 25 mm to 75 mm. The term “diameter” as used in this disclosure does not require that a coil/turn be a complete or perfectly-shaped circle but is generally used to refer to a greatest width across opposing points of the coil/turn. For example, with respect to the leading coil/turn, diameter can be measured from the distal tip 84 to the opposite side, as if the lower region or leading coil/turn 82 formed a complete rotation.

In various examples, the docking device 70 can also include an enlarged proximal or upper region 86 that comprises and/or consists of a stabilizing coil/turn (e.g., which can be an atrial coil/turn) of the docking device 70. During a transient or intermediate stage of the implantation procedure, that is, during the time between the deployment and release of the docking device 70 and final delivery of the prosthetic valve, there is a possibility that the coil could be shifted and/or dislodged from its desired position or orientation, for example, by regular heart function. Shifting of the docking device 70 could potentially lead to a less secure implantation, misalignment, and/or other positioning issues for the prosthetic valve. A stabilization feature or coil can be used to help stabilize the docking device in the desired position. For example, the docking device 70 can include the upper region 86 with an enlarged stabilization coil/turn (e.g., an enlarged atrial coil/turn having a greater diameter 92 and/or 94 than the functional coils) intended to be positioned in the circulatory system (e.g. in the left atrium) such that it can stabilize the docking device. For example, the upper region 86 or stabilization coil/turn can be configured to abut or push against the walls of the circulatory system (e.g., against the walls of the left atrium), in order to improve the ability of the docking device 70 to stay in its desired position prior to the implantation of the prosthetic valve.

The stabilization coil/turn (e.g., atrial coil/turn) at the upper region 86 of the docking device 70 in the examples shown can extend up to about one full turn or rotation, and terminates at a proximal tip 88. In other examples, the stabilization coil/turn (e.g., atrial coil) can extend for more or less than one turn or rotation, depending for example on the amount of contact desired between the docking device and the circulatory system (e.g., with the walls of the left atrium) in each particular application. The radial size of the stabilization coil/turn (e.g., atrial coil) at the upper region 86 can also be significantly larger than the size of the functional coils in the central region 80, so that the stabilization coil/turn (e.g., atrial coil or atrial turn) flares or extends sufficiently outwardly in order to contact the walls of the circulatory system (e.g., the walls of the left atrium). In some examples, a core diameter in the upper region 86 can be varied which can result in this section of the coil being more or less stiff than other sections of the coil and/or conforming to the anatomy. Additionally, the stabilization coil/turn of various examples will be configured to be less abrasive to the native tissue and/or anatomy. For example, the surface texture can be made smoother and/or softer, such that movement of the docking device against the native anatomy will not damage the native tissue.

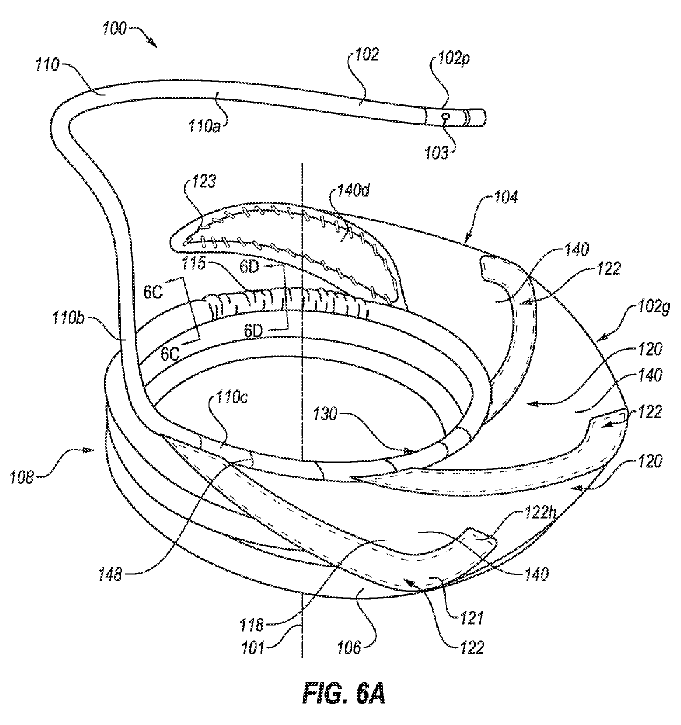

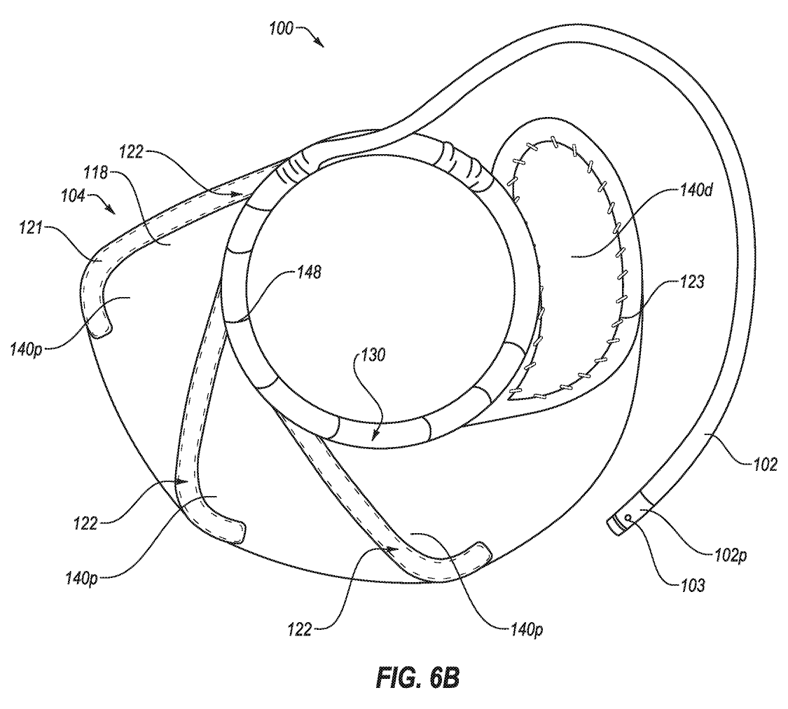

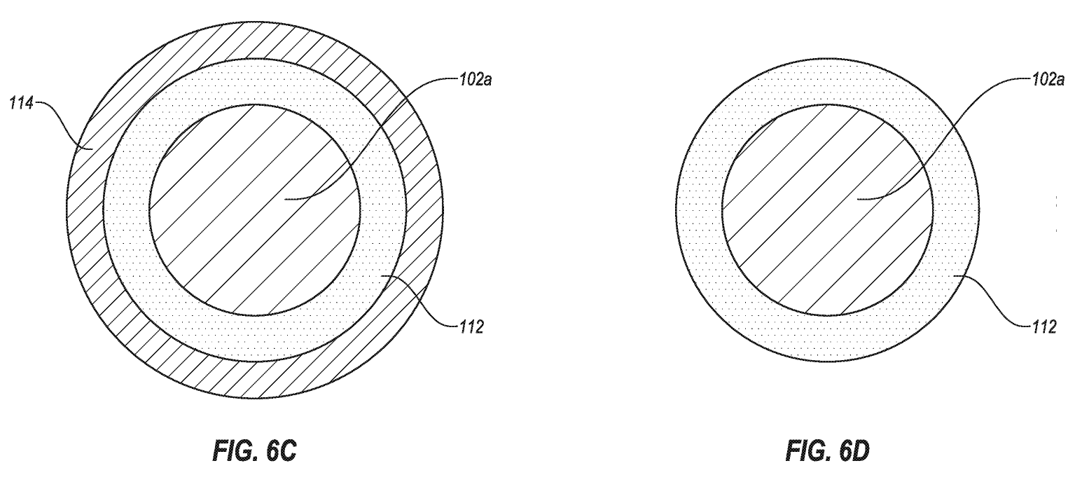

FIG. 5B illustrates a scaffold 120 of a guard member 104 (described herein, FIGS. 6A-6B) that attaches to the coil 102 of the docking device 70. The scaffold 120 includes a spine 130 with a plurality of arms 122 extending therefrom. The arms 122 include a base portion 122b and a head portion 122h. The scaffold 120 includes gaps 127 between the arms 122, where the gaps are used as panels 140 of the guard member 104. The first set of arms 122 can be linear arms as shown. The last arm 125 can be a lobe, also referred to as terminal lobe 125. In some aspects, the arms 122 can have varying widths from the base portion 122b to the head portion 122h, where a narrowing taper of the width from the base portion 122b to the head portion 122h can provide for favorable characteristics when deploying the guard member. That is, the base portion 122b can be wider than the arm 122 near the head portion, where the thickness can be constant. However, the width could be constant and the thickness could narrow from the base portion 122b to the head portion 122h. In some examples, the base portion 122b can be a width in a range of 0.2 mm to 0.3 mm. In some examples the arm 122 near the head portion 122h can comprise a width in a range of 0.07 mm to 0.15 mm. In some examples, the arms may vary in width from 0.25 mm at the base portion 122b to 0.12 mm at the arm 122 near the head portion 122h. This tapered arm 122 may provide adequate retention strength while allowing the arms to better conform to the native anatomy of the valve region.

The guard member 104 can be configured to fit at the mitral position over the mitral valve to provide a cover over the mitral leaflets and perimeter of the mitral valve region. However, the guard member 104 can be shaped and/or adapted similarly or differently in other examples for better accommodation at other native valve positions as well, such as at the tricuspid valve, which can be configured along with the docking device 70. Advantageously, the guard member 104 geometries of the present disclosure provide for engagement with the native anatomy at the mitral valve that can provide for increased stability and reduction of relative motion between the docking device 70 and/or guard member 104 with respect to the native anatomy. Reduction of such relative motion can prevent gaps to form to allow for leakage around the guard member 104 and can prevent damage/trauma to the native tissues. Accordingly, the guard member 104 can be configured to provide an adaptive fit to the docking device 70 to inhibit movement relative to the mitral valve anatomy and inhibit leakage of blood, such as inhibit PVL. Accordingly, the number of arms 122 as well as the length, thickness, and head features can be modulated for different sized anatomies, such as from children through adults, and the various sizes thereof. Also, the flexibility of the guard member 104 due to the flexibility of the arm 122 can contribute with shaping and contouring of the guard member 104 with the adjacent anatomy at the mitral valve. The thickness of the scaffold, or spine or arms thereof can be varied in dimension to be bigger at the base of the arm and narrower at the head or end of the arm.

The guard member 104 can include the spine 130 in a shape that corresponds with the coil 102 of the docking device 70, such that both the spine 130 and coil 102 have substantially the same coil or diameter so that the bodies thereof match and can be coupled together. For example, the spine 130 from one end to the other can be cooperative with a region of the coil 102 such that they fit together and have the same curvature, such as without gaps when the spine 130 is placed on the coil 102.

In examples where the docking device 70 is used at the mitral position, the docking device 70 with the guard member 104 can first be advanced and delivered to the native mitral valve annulus, and then set at a desired position with the guard member covering the mitral leaflets and perimeter anatomy, prior to implantation of the prosthetic heart valve. In some examples, the guard member 104 is flexible and/or made of a shape memory material, so that the spine 130 coils with the docking device 70 and can be straightened for delivery via a transcatheter approach as well. In some examples, the scaffold 120 is made of shape memory material (e.g., nitinol) or another biocompatible material, such as stainless steel. Some of the same catheters and other delivery tools can be used for both delivery of the docking device 70 with the guard member 104 and the prosthetic valve, without having to perform separate preparatory steps, simplifying the implantation procedure for the end user.

Since spine 130 is configured to be shaped to match the coils/turns of the docking device 70, such as at the central region 80, the effective diameter of the spine 130 can be kept relatively small in diameter (e.g., to match the central region 80 in one example can have an inner diameter of between approximately 21-24 mm ± 2 mm or another diameter smaller than the prosthetic valve and/or the native annulus) in order to increase retention force with the prosthetic valve. Also, the guard member 104 can be placed at a location on the central region 80 where it inhibits further advancing of the docking device 70 around the existing leaflets and/or chordae tendineae, and guard member 104 is shaped to help deliver the docking device 70 to a desired position relative to the native mitral annulus.

The spine 130 can include a leading end 132 and a trailing end 134 with a concave side 136 therebetween. The concave side 136 can be shaped to match the coil 102 of the docking device 70. The arms 122 extend from a convex side 138 of the spine 130, and thereby away from the coil 102 of the docking device 70. While the spine 130 can be shaped to match the coil 102, the spine 130 may or may not be directly coupled with the coil 102. In some examples, the coil 102 can include a material that can be coupled with the material of the spine 130, such as be sewing, stitching, suturing, brazing, welding, adhesive, or the like. In some examples, the docking device 70 may include a material cover around the base coil, and the spine 130 may also include a material cover (e.g., flap 118) that can be coupled with the cover of the coil. That is, the covers of the two components can be coupled together, such as by suturing, sowing, adhesive, clipping, or otherwise affixing the guard member 104 to the docking device 70, which is discussed in more detail herein. For example, the guard member 104 can be stitched to the coil 102 with sutures to couple the guard member to the coil.

The length of the spine 130 can be modulated depending on the design of the docking device 70. Accordingly, the spine 130 can be configured to cover a certain percentage of a full coil turn or even a full 360 degree turn or more. The spine length can be tailored so that is matches with the mitral valve anatomy and provides a sufficient length for arms 122 extending therefrom to engage and overlap the anatomy to provide a cover. The length of the spine 130 can be relative to the central region 80 of the docking device 70. In some aspects, the length of the spine 130 from the leading end 132 to the trailing end 134 can be from about 20 mm to about 150 mm, from about 30 mm to about 100 mm, from about 40 mm to about 90 mm, from about 45 mm to about 80 mm, or about 50 mm to about 75 mm. In some examples, the length can be about 54 mm.

The thickness of the scaffold 120 can also be varied as needed or desired. When thickness is being referred to, the Z dimension relative to the X-Y area of the page. The thickness is the height if the scaffold 120 is laid on its side with the arms 122 extending across the horizontal plane. The thickness can range from about 0.1 mm to about 0.8 mm, from about 0.2 mm to about 0.6 mm, from about 0.3 mm to about 0.5 mm, from about 0.4 to about 0.45 mm. In some aspects, the spine 130 and the arms 122 can have the same thickness. In other aspects, the spine 130 may have a larger thickness compared with the arms 122.

The width of the spine 130 and arms 122 can also vary. The width, which is orthogonal with the thickness, defines dimension in the X-Y plane of the component. The width of the spine 130 is between the concave side 136 and the convex side 138. The corresponding dimension of the arms 122 is also considered the width. The width of the spine 130 and/or arms 122 can independently range from about 0.05 mm to about 0.5 mm, from about 0.1 mm to about 0.4 mm, from about 0.13 mm to about 0.3 mm, from about 0.16 to about 0.25 mm, or from about 0.15 mm to about 0.20 mm. In a tapered example, the arm can taper from about 0.35 mm to about 0.05 mm, or from about 0.25 mm to about 0.1 mm.

The arms 122 can be distributed along the convex side 138 of the spine 130 as shown in FIG. 5B. The arms 122 are shown to have the base portion 122b attached to the spine 130 with the head portion 122h on the opposite end of the arms 122. The arm 122 can include a straight portion 122c extending from the base portion 122b for a certain length, which then turn into an arc 122a that bends the arm 122 in a bend region 122d around so that the arm 122 has the bend region 122d that is somewhat parallel with the spine 130. That is, the arc 122a turns the direction of the arm 122 so that the bend region 122d is oriented in about the same direction as the spine 130. Also, it is noted that the bend region 122d and head portion 122h are oriented away from the leading end 132 and pointed toward the trailing end 134.

The arms 122 can range from about four arms to about ten arms, from about 3 arms to about 20 arms, or about 4 arms to about 15 arms, or about 5 arms to about 10 arms, or about 6-8 arms. The arms 122 can vary in length from the base of the base region 122b (e.g., from spine 130) to the tip of the head region 122h from about 10 mm to about 60 mm, from about 20 mm to about 50 mm, from about 25 mm to about 45 mm, from about 30 mm to about 43 mm, or about 35 mm to about 40 mm. The straight portion 122c can have a length of about 6 mm to about 50 mm, from about 8 mm to about 40 mm, from about 10 mm to about 30 mm, from about 15 mm to about 20 mm. The arc 122a can have an angle from about 20 degrees to about 90 degrees, from about 30 degrees to about 80 degrees, from about 40 degrees to about 70 degrees, from about 50 degrees to about 60 degrees. The bend region 122d and the head region 122h may be the dimension of the arm 122 minus the dimension straight portion 122c. However, the lengths of the arm can vary across different examples or across the different arms of the same scaffold. In some aspects, the size of the scaffold component is larger in diameter than the coil of the docking station. When the guard member is attached to the docking station, the guard member has more radial coverage with the flap of the guard member. Also, the flap member of the guard member is cut larger than the docking station (e.g., 33 mm diameter of flap as compared to the dock diameter of 29 mm after implant) so the textile materials of the flap are stretched tight during attachment, mitigating wrinkles in the textiles.

The arms 122 can be separated from each other by a dimension of about 0.3 mm to about 15 mm, from about 0.75 mm to about 10 mm, from about 1 mm to about 8 mm, from about 1.25 mm to about 6 mm.

The head portion 122h may also be referred to as the head 122h herein. As such, the head 122h can have a rounded shape with or without an aperture. The head 122h can include a loop 122l shape that defines an aperture 122k, which dimensions can vary. The loop 122l and aperture 122k is shown to have a teardrop shape; however, the shape can be completely circular, oval, or other variation of roundedness. The head 122h can be configured so that it does not have any sharp ends or points, which can minimize puncturing of the flap 118 or the mitral valve tissue or related anatomy. The head 122h provides for a rounded feature that is blunted to inhibit any puncturing.

The scaffold 120 may also have a terminal lobe 125, which may also be referred to as a petal herein or in the incorporated references. However, two terminal lobes 125 can be placed on the scaffold, with one at each end. The terminal lobe 125 is shown to have two ends 125a, 125b attached to the spine 130 to form the loop 125l and aperture 125k. However, only a single end may be attached to the spine 130, such as at or near the trailing end 134. The terminal lobe 125 can have various dimension and may be oblong or somewhat teardrop shaped. The terminal lobe 125 can have a length of about 5 mm to about 50 mm, from about 10 mm to about 40 mm, or from about 20 to about 30, or about 21 mm. The terminal lobe 125 can have a width from about 3 mm to about 30 mm, from about 5 to about 25 mm, from about 10 mm to about 20 mm, or from about 11 mm to about 15 mm, or about 10.5 mm.

In some examples, the scaffold 120 can include a shape memory material that is shape set and/or pre-configured to expand the guard member 104 to the radially expanded state when unconstrained (for example, when deployed at a native valve location). For example, the scaffold 120 can contain a shape memory alloy with super-elastic properties, such as Nitinol. In some examples, the scaffold 120 can contain a ternary shape memory alloy with superelastic properties, such as NiTiX where X can be chromium (Cr), cobalt (Co), zirconium (Zr), hafnium (Hf), etc.

In some examples, the scaffold 120 can comprise a metallic material that does not have the shape memory properties. In such circumstances, the scaffold 120 can have a biasing mechanism (for example, using springs, etc.) configured to bias the scaffold 120 (and the guard member 104) to the radially expanded state. Examples of such metallic material include cobalt-chromium, stainless steel, etc. In one specific example, the scaffold 120 can comprise nickel-free austenitic stainless steel in which nickel can be completely replaced by nitrogen. In another specific example, the scaffold 120 can comprise cobalt-chromium or cobalt-nickel-chromium-molybdenum alloy with significantly low density of titanium.

FIG. 5C illustrates a flap 118 of the guard member 104, as described herein. The flap 118 is adapted to fit over the scaffold 120 to form the guard member 104. The flap 118 can include a at least one flap sheet 150 that has sleeves 121 formed of sleeve sheet 152 coupled with the flap sheet 150, such as with sheet stiches 154, adhesive, or other attachment means. The flap 118 is shown to include five panels 140 and one terminal panel 140a configured as a terminal petal, with a petal shape (e.g., teardrop-like shape). Here, the flap sheet 150 can be one or more sheets, and may be configured as at least two sheets coupled (e.g., stitched) together to form a cover that with a cavity slips over the arms to cover both sides so that the arms are in the cavity between the sheets. Any example of the one or more flap sheets 150 is considered herein. In some aspects, the sleeves 121 can be tubes, such as braded tubes, which can fit over the arms like a sock. Other sleeve configurations can also be used.

In some examples, the flap 118 of FIG. 5C is fit onto the scaffold 120 of FIG. 5B to form the guard member 104. The guard member is then coupled to the coil 102 of FIG. 5A to form the docking device having the guard member 104.

The flap 118 can be a single flap sheet 150 or a plurality of flap sheets 150 affixed to the scaffold 120 by any means. The affixing can be via the flap 118 being sutured to the scaffold 120, such as by loop stitches 156. Alternatively, another sheet, whether flat or tubular (e.g., sock), can be configured as a sleeve 121 that receives the arms 122 and is coupled with the flap sheet 150 via sheet stiches. Accordingly, different coupling systems can be used to couple the flap sheet 150 to the scaffold 120.

In some examples, at least one arm 122 is coupled to the flap 118 by having the flap sheet 150 coupled with a sleeve sheet 152 with the arm 122 therein with sheet stiches 154 coupling the flap sheet 150 to the sleeve sheet 152. In some aspects, the flap 118 is a flat sheet of material, such as a fabric, film, membrane, plastic sheet, foil, or the like. In some aspects, the sleeve sheet 152 is a flat sheet of material, which can be the same or different material from the flap 118. In a combination of the flat flap sheet 150 and the flat sleeve sheet 152, the arm 122 is fit between the flat flap sheet 150 and flat sleeve sheet 152 with sheet stitches 154 stitching each side of the arm 122 to form the sleeve 121. The arm 122 is able to freely move inside of the sleeve component that protects the arms, which enhances the ability to compress the guard member into the catheter tube.

In some examples, at least one arm 122 is coupled to the flap 118 by having the sleeve sheet 152 formed as a tube (e.g., two open ends) or sock (e.g., one open end) slipped over the arm 122 and stitched (e.g., 154) to the flap sheet 150. Here, the sleeve sheet 152 encapsulates the arm 122 to provide additional protection, which can be beneficial to the mitral valve tissue. The tubular or sock sleeve sheet 152 can also be made from the same materials as the flat flap sheet 150, but may or may not be the same material in a particular example.

In some examples, at least one arm 122 is coupled to the flap 118 by having loop stiches 156 stitching a single arm 122 to a flap sheet 150. The loop stiches 156 can go through the flap sheet 150 and around the respective arm 122 and back through the flap sheet 150 on the other side of the arm 122 to form a looping stitch around the arm 122. Various types of loop stiches 156 can be used so long as the stitching forms a loop coupling the arm 122 to the flap sheet 150. For example, the terminal lobe 125 is basically an arm with both ends coupled to the spine 130. As such, the terminal lobe 125 may not be adapted to receive the tube or sock sleeve configuration. Also, such a terminal lobe 125 does not have any sharp points, ends or edges. Accordingly, the arm of the terminal lobe 125 can be loop stitched to the flap sheet 150. In some aspects, the guard member can include stitching around the base of the terminal lobe (e.g., loop), whereas at the end of the terminal lobe it is only stitched on the inner diameter. Therefore, the lobe can move within the flap member to allow the terminal lobe to collapse into the catheter.

In some examples, at least one arm 122 is coupled to the flap 118 by having loop stiches 156 stitching a single arm 122 between a flap sheet 150 and flat sleeve sheet 152, where loop stiches 156 are used. The loop stiches 156 can go through the flap sheet 150 and the flat sleeve sheet 152 around the respective arm 122 and back through the flat sleeve sheet 152 and flap sheet 150 on the other side of the arm 122 to form a looping stitch around the arm 122 and the flap sheet 150 and flat sleeve sheet 152. Various types of loop stiches 156 can be used so long as the stitching forms a loop coupling the arm 122 to the flap sheet 150.

When the arm is the terminal lobe 125, the flat sleeve sheet 152 can be configured as a flat lobe sheet 158. The flat lobe sheet 158 can be loop stitched with the terminal lobe 125 between the flap sheet 150 and flat lobe sheet 158. As shown in FIG. 5C, the flap sheet 150 is loop stitched via the loop stiches 156 to the flat lobe sheet 158 with the terminal lobe 125 therebetween. In some aspects, the flat lobe sheet 158 can be configured as a terminal petal, with a petal shape (e.g., teardrop-like shape), as shown in FIG. 5C.

As described, the guard member 104 is shown to include five panels 140 and one terminal panel 140a formed from the scaffold 120 and the flap 118. The panels 140 can be regions of the flap sheet 150 between the arms 122. The panels 140 can function as flat umbrella panels that can fold up when the scaffold 120 is folded into a delivery orientation and then expand once the scaffold 120 is released into a deployed orientation. The panels 140 can be various shapes and sizes for different configurations. The panels 140 extend from the spine 130 out past the arms 122 to provide a brim feature with respect to the delivery device 70. The panels 140 can provide a barrier that is flexible and can contour with the mitral valve anatomy. The panels 140 can inhibit fluid flow from passing the guard member 104, and thereby can function to guard against paravalvular leakage. The panels 140 may also allow for cellular ingrowth depending on the type of material, which can facilitate implantation and longevity of beneficial function.

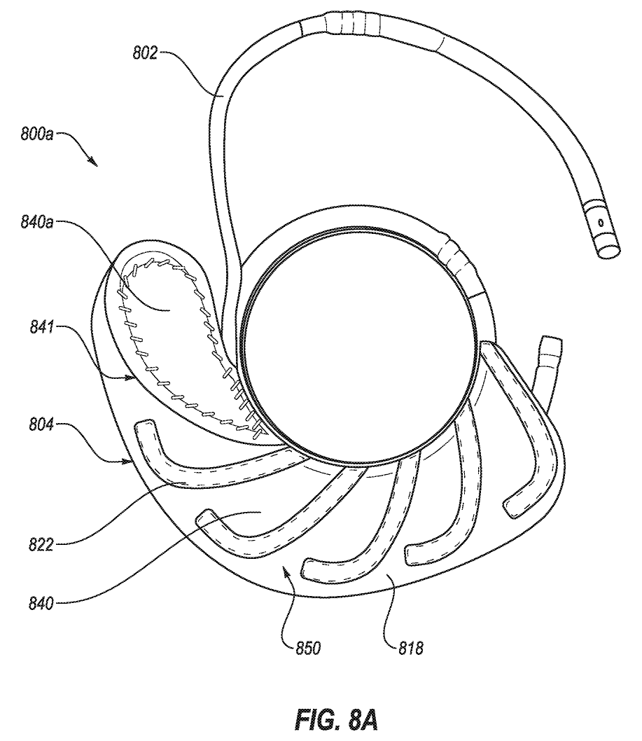

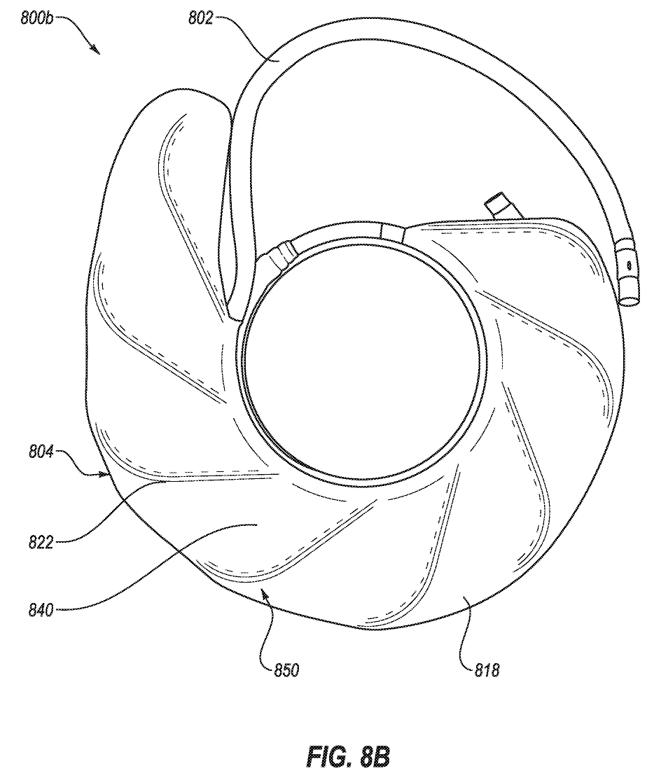

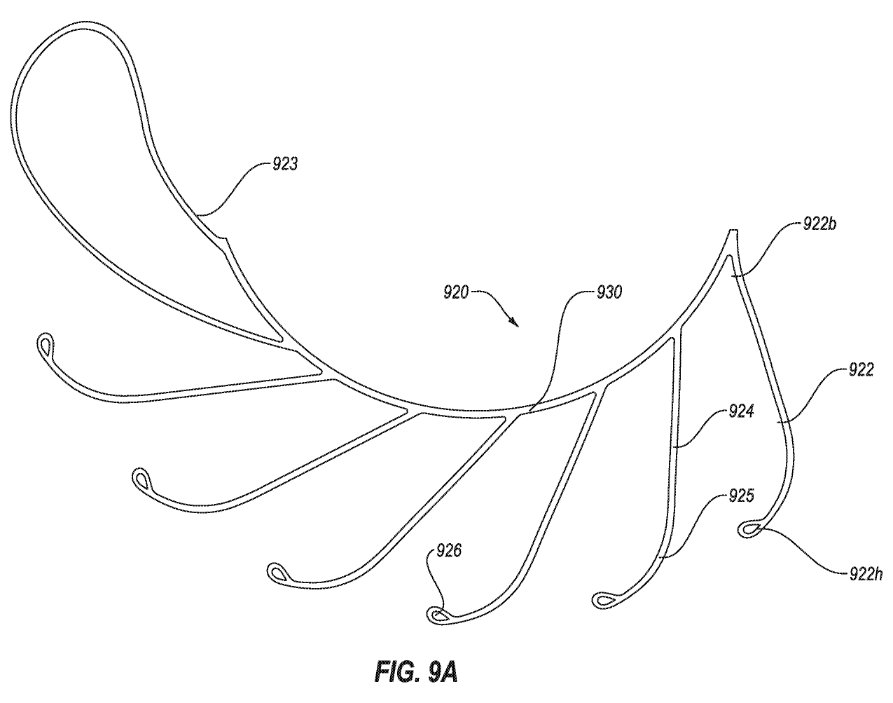

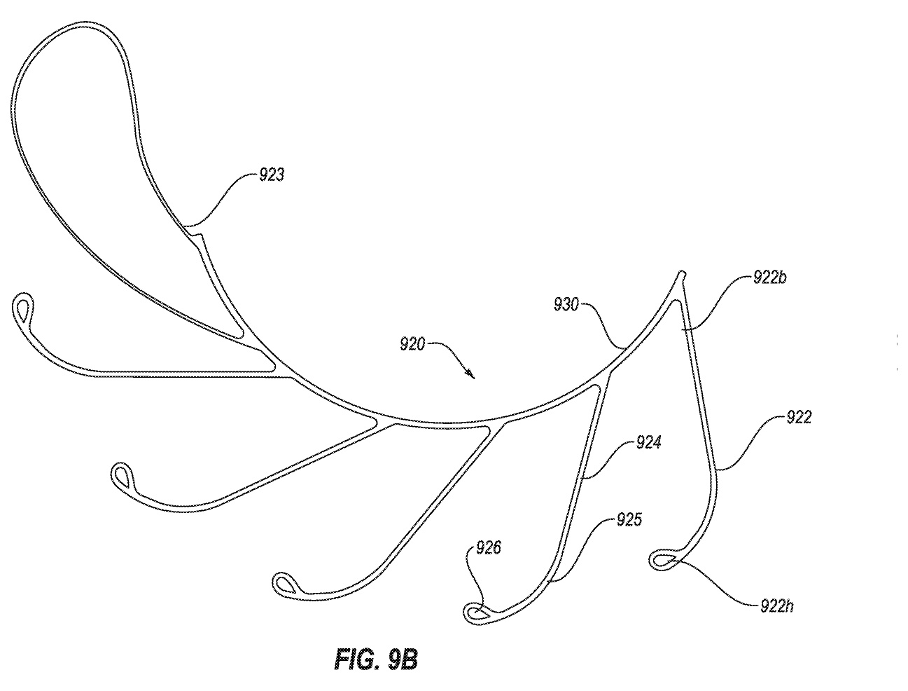

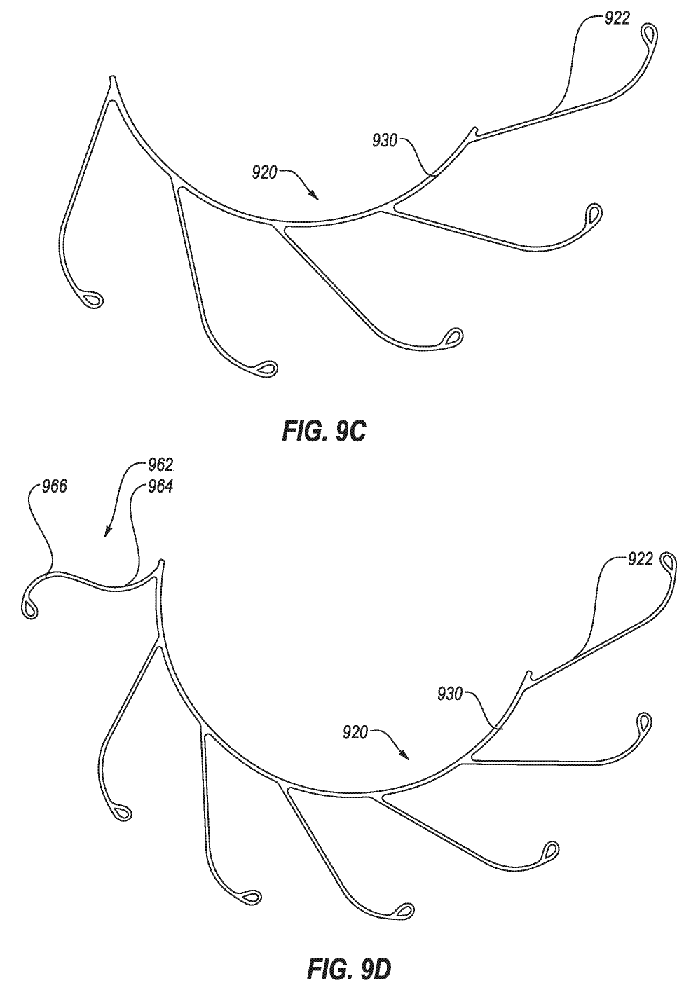

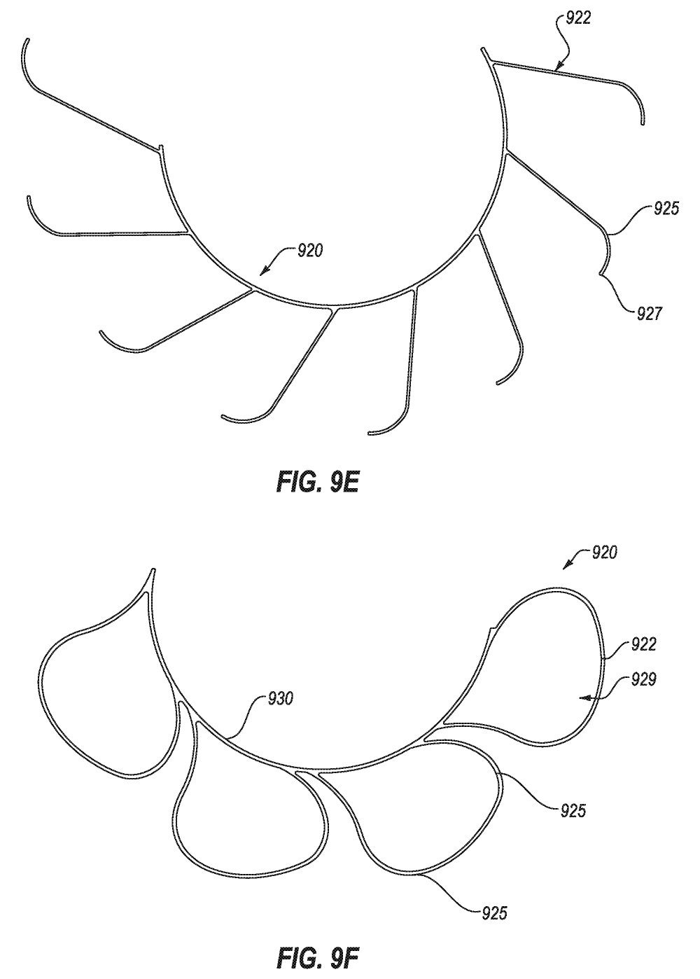

As shown in FIGS. 5B-5C, the arms 122, sleeves 121, terminal lobe 125 and lobe sleeve 158 can be oriented clockwise or counter-clockwise in the deployed orientation. That is, these component can be oriented with respect to the coil 102 of the docking device 70 as illustrated in clockwise deployment or in the opposite direction in counter-clockwise deployment. While all of the arms 121 and terminal lobe 125 are oriented in the same direction as illustrated, the terminal lobe 125 may be oriented in the opposite direction (e.g., counter-clockwise) while the arms 121 are still in clockwise orientation. Additionally, one or more arms 121 may be substituted with lobes, which can be internal lobes. Also, the terminal lobe 125 can be substituted with a terminal arm. Therefore, various configurations can be provided, such as shown in FIGS. 8A-8B, and FIGS. 9A-9F, and described in more detail herein.

The flap 118 may also be coupled with the spine 130 of the scaffold 120. The flap 118 may be affixed with the spine 130 in a similar manner as the flap sheet 150 is fixed to an arm 122. The flap 118 can be loop stitched with the spine 130 so that the flap 118 covers the spine 130. The stitching can also secure the guard member to the coil member. In another aspect, the flap sheet 150 can be looped around the spine itself and then stitched or loop stitched, which forms an interrupted tubular covering of the flap sheet 150 around the portions of the spine 130 between the arms 122, which interrupted tubular cover can be referred to as a spine sleeve 153. In some aspects, another sheet material can be used for forming the spine sleeve 153, which can be performed similar to the arm as described herein.

In some examples, the flap 118 can be configured to be so elastic that when the guard member 104 moves from the delivery orientation to the deployed orientation, the flap 118 can accommodate the scaffold 120.

In some examples, the flap 118 can be configured to be atraumatic to native tissue and/or promote tissue ingrowth into the flap 118. For example, the flap 118 can have pores to encourage tissue ingrowth. In another example, the flap 118 can be impregnated with growth factors to stimulate or promote tissue ingrowth, such as transforming growth factor alpha (TGF-alpha), transforming growth factor beta (TGF-beta), basic fibroblast growth factor (bFGF), vascular epithelial growth factor (VEGF), and combinations thereof. The flap 118 can be constructed of any suitable material, including foam, cloth, fabric, and/or polymer, which is flexible to allow for compression and expansion of the flap 118. In one example, the flap 118 can include a fabric layer constructed from a thermoplastic polymer material, such as polyethylene terephthalate (PET).

In some examples, the flap 118 can be configured to engage with the prosthetic valve deployed within the docking device so as to form a seal and reduce paravalvular leakage between the prosthetic valve and the docking device after the guard member 104 is radially expanded. The flap 118 can also be configured to engage with the native tissue (for example, the native annulus and/or native leaflets) to reduce PVL between the docking device and/or the prosthetic valve and the native tissue.

Additionally, the flap 118 can include an edge protector 151 at a peripheral lip that is the region peripheral to the arms 122 and/or sleeves 121. The edge protector 151 can be a part of the flap sheet 150 or a separate member coupled with the flap sheet 150. Additional examples of the flap 118 are described herein.

FIGS. 6A-6D show a docking device 100 with a coil 102 coupled to a guard member 104, according to one example. The docking device 100 can, for example, be implanted within a native valve annulus. The docking device 100 can be configured to receive and secure a prosthetic valve (for example, prosthetic heart valve 62), thereby securing the prosthetic valve at the native valve annulus.

The docking device 100 can comprise a coil 102 and a guard member 104 (which can also be referred to as “a PVL guard” or “a sealing member” or a “brim feature”) extending along at least a portion of the coil 102. In certain examples, the coil 102 can include a shape memory material (for example, nickel titanium alloy or Nitinol) such that the docking device 100 (and the coil 102) can move from a substantially straight configuration (also referred to as “delivery orientation”) when disposed within a delivery sheath of a delivery apparatus (for example, docking device delivery apparatus 50) to a helical configuration (also referred to as “deployed orientation,” as shown in FIG. 6A) after being removed from the delivery sheath. In the delivery orientation, the arms 122 of the guard member 104 are folded up against the spine 130 so that the panels are folded inward. In the deployed orientation, the arms 122 are released and extend out away from the spine 130, which provides a brim feature for covering the mitral valve anatomy.

During delivery of the docking device 100 and after initial deployment of the docking device 100 at the implantation site, the guard member 104 can be retained in a radially compressed state by a dock sleeve of the delivery apparatus. After the docking device 100 is deployed at the implantation site, the dock sleeve can be removed so as to expose the guard member 104, thereby allowing the guard member 104 to move to a radially expanded state, such as in FIGS. 6A-6B.

In some examples, when the docking device 100 is in the deployed orientation and the guard member 104 is in the radially expanded state, the guard member 104 can extend circumferentially, radially, or laterally relative to a central longitudinal axis 101 of the docking device 100. The guard member 104 can extend around the circumference of a turn in the coil 102 from 180 degrees to 400 degrees, or from 210 degrees to 330 degrees, or from 250 degrees to 290 degrees, or from 260 degrees to 280 degrees (for example, 270 degrees) relative to the central longitudinal axis 101. In other words, the guard member 104 can extend circumferentially from about one half of a revolution (for example, 180 degrees) around the central longitudinal axis 101 in some examples to more than a full revolution (for example, 400 degrees) around the central longitudinal axis 101 in other examples, including various ranges in between. As used herein, a range (for example, from 180 degrees to 400 degrees, and between 180 degrees and 400 degrees) includes the endpoints of the range (for example, 180 degrees and 400 degrees), as will all ranges recited herein being inclusive of the endpoints. In some aspects, the guard member can achieve at least 360 degrees of coverage of the valve anatomy in the atrium. The unfolding of the guard member allows for such coverage. Examples can include about 45 degrees of coverage to about 400 degrees of coverage, about 90 degrees of coverage to about 360 degrees of coverage, about 120 degrees of coverage to about 300 degrees of coverage, or about 180 degrees of coverage to about 225 degrees of coverage.

The coil 102 has a proximal end 102p and a distal end 102g, with the guard member 104 therebetween, which also respectively define the proximal and distal ends of the docking device 100. When being disposed within the delivery sheath (for example, during delivery of the docking device into the vasculature of a patient), a body of the coil 102 between the proximal end 102p and distal end 102d along with the guard member 104 can form the generally straight delivery orientation (that is, without any coiled or looped portions, but can be flexed or bent) so as to maintain a small radial profile when moving through a patient’s vasculature. After being removed from the delivery sheath and deployed at an implant position, the coil 102 and guard member 104 can move from the delivery orientation to the helical deployed orientation with the guard member 104 extended laterally from the coil on top of the mitral valve anatomy, and with the coil wrapping around native leaflet tissue adjacent the implant position. For example, when implanting the docking device at the location of a native valve, the coil 102 can be configured to surround native leaflets of the native valve (and the chordae tendineae that connects native leaflets to adjacent papillary muscles, if present) with the guard member 104 on top so as to be over where the leaflets would be, thereby the guard member 104 forming a brim or flap over the mitral valve anatomy in the left atrium.

The docking device 100 can be releasably coupled to a delivery apparatus (for example, docking device delivery apparatus 50). For example, in certain examples, the docking device 100 can be coupled to the delivery apparatus via a release suture that can be configured to be tied to the docking device 100 and cut for removal. In one example, the release suture can be tied to the docking device 100 through an eyelet or eyehole 103 located adjacent the proximal end 102p of the coil. In another example, the release suture can be tied around a circumferential recess that is located adjacent the proximal end 102p of the coil 102.

In some examples, the docking device 100 in the deployed orientation can be configured to fit at the mitral valve position with the guard member 104 covering the mitral anatomy laterally from the coil 102 in the left atrium. The guard member 104 can provide a lateral barrier on a peripheral of the mitral valve anatomy in the left atrium intersection with the mitral valve anatomy. In other examples, the docking device 100 can also be shaped and/or adapted for implantation at other native valve positions as well, such as at the tricuspid valve. As described herein, the geometry of the docking device 100 and guard member 104 thereof can be configured to engage the native anatomy, which can, for example, provide for increased stability and reduction of relative motion between the docking device 100, the prosthetic valve docked therein, and/or the native anatomy. Reduction of such relative motion can, among other things, prevent material degradation of components of the docking device 100 and/or the prosthetic valve docked therein and/or prevent damage or trauma to the native tissue. Also, the guard member 104 can inhibit paravalvular leaking of blood the wrong direction in the valvular pathway.

As shown in FIG. 6A, the coil 102 in the deployed orientation can include a leading turn 106 (or “leading coil”), a central region 108 (e.g. having the guard member 104), and a stabilization turn 110 (or “stabilization coil”) around the central longitudinal axis 101. The central region 108 can possess one or more helical turns having substantially equal inner diameters with one of these turns being coupled with the guard member 104. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations). However, the diameters can be the same in other configurations.

In some examples, the central region 108 can include a plurality of helical turns (for example, the docking device 100 can have three helical turns in the central region 108). Some of the helical turns in the central region 108 can be full turns (that is, rotating 360 degrees). In some examples, the most proximal turn and/or the most distal turn can be partial turns (for example, rotating less than 360 degrees, such as 180 degrees, 270 degrees, etc.). The guard member 104 can be positioned anywhere along the central region 108, and is shown at the proximal turn thereof.

In some examples, the top-most or most proximal helical turn of the central region 108 can include the guard member 104 coupled thereto. This provides the guard member 104 in the region of the coil 102 that is in the left atrium, while the distal helical turns go into the left ventricle around the leaflets.

The size of the docking device 100 and guard member 104 can be generally selected based on the size of the desired prosthetic valve to be implanted into the patient and the size of the anatomy at the left atrium intersection with the mitral valve anatomy. In some examples, the central region 108 can be configured to retain a radially expandable prosthetic valve. For example, the inner diameter of the helical turns in the central region 108 can be configured to be smaller than an outer diameter of the prosthetic valve when the prosthetic valve is radially expanded so that additional radial force can act between the central region 108 and the prosthetic valve to hold the prosthetic valve in place. The helical turns in the central region 108 can also be referred to herein as “functional turns.” The spine 130 of the guard member 104 can be similarly configured for this intended use.

The stabilization turn 110 can be configured to help stabilize the docking device 100 in the desired position. For example, the radial dimension of the stabilization turn 110 can be significantly larger than the radial dimension of the coil in the central region 108, so that the stabilization turn 110 can flare or extend sufficiently outwardly so as to abut or push against the walls of the circulatory system, thereby improving the ability of the docking device 100 to stay in its desired position prior to the implantation of the prosthetic valve. In some examples, the diameter of stabilization turn 110 is desirably larger than the native annulus, native valve plane, and/or native chamber for better stabilization. In some examples, the stabilization turn 110 can be a full turn (that is, rotating about 360 degrees). In some examples, the stabilization turn 110 can be a partial turn (for example, rotating between about 180 degrees and about 270 degrees). In some aspects, the guard member 104 can be opposite of the stabilization turn 110 such that they push out laterally in opposite directions.

In one particularly example, when implanting the docking device 100 at the native mitral valve location, the functional turns in the central region 108 can be disposed substantially in the left ventricle and the stabilization turn 110 can be disposed substantially in the left atrium above the guard member 104. The stabilization turn 110 can be configured to provide one or more points or regions of contact between the docking device 100 and the left atrial wall, such as at least three points of contact in the left atrium or complete contact on the left atrial wall opposite of the guard member contacting the left atrial wall. In some examples, the points of contact between the docking device 100 and the left atrial wall can form a plane that is approximately parallel to a plane of the native mitral valve, and may be parallel to a plane of the guard member 104. In some aspects, the guard member can be configured to stabilize the docking device into the mitral valve. As such, the device can omit an atrial turn in the coil.

In some examples, the stabilization turn 110 can have an atrial portion 110c (attached to the guard member 104 in FIG. 6A) in connection with the central region 108, a raised stabilization portion 110a adjacent to the proximal end 102p of the coil 102, and an ascending portion 110b located between the atrial portion 110c and the raised stabilization portion 110a. The ascending portion 110b can be adjacent with the guard member 104. Both the atrial portion 110c and the raised stabilization portion 110a can be generally parallel to the helical turns in the central region 108, whereas the ascending portion 110b can be oriented to be angular relative to the atrial portion 110c and the raised stabilization portion 110a. For example, in certain examples, the ascending portion 110b and the raised stabilization portion 110a can form an angle from about 45 degrees to about 90 degrees (inclusive). When implanting the docking device 100 at the native mitral valve location, the atrial portion 110c can be configured to abut against a posterior wall of the left atrium and the raised stabilization portion 110a can be configured to flare out and press against an anterior wall of the left atrium, along with the guard member.

In some examples, the coil 102 can omit the stabilization turn, and the proximal region of the coil 102 can be another portion coil 102. In this example, the guard member 104 provides the stabilization of the docking device with respect to the mitral valve anatomy and the left ventricle.

As noted above, the leading turn 106 can have a larger radial dimension than the helical turns in the central region 108. The leading turn 106 can help more easily guide the coil 102 around and/or through the chordae tendineae and/or adequately around all native leaflets of the native valve (for example, the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (that is, rotating about 360 degrees). In some examples, the leading turn 106 can be a partial turn (for example, rotating between about 180 degrees and about 270 degrees). When a prosthetic valve is radially expanded within the central region 108 of the coil, the functional turns in the central region 108 can be further radially expanded. As a result, the leading turn 106 can be pulled in the proximal direction and become a part of the functional turn in the central region 108.

In some examples, at least a portion of the coil 102 can be at least partially surrounded by a cover. The cover can, for example, prevent or reduce trauma to native tissue and/or prevent or reduce damage to the delivery device, reduce friction with the native tissue, increase friction with the native tissue and/or prosthetic heart valve, etc. In some instances, the coil can comprise a plurality of covers and/or a plurality of sections of one or more covers, each configured for a particular purpose. For example, a first cover can be provided over all or at least substantially all of the coil, for example, to prevent or reduce trauma to the native tissue. A second cover can extend over a portion of the first cover and can, for example, be configured to increase friction between the cover and native leaflet tissue. Additional information about the covers is provided below and can be found in International Publication No. WO 2022/087336. This cover can be used to couple the guard member 104 to the coil. As such, material of the guard member 104 can be coupled to material of the cover, such as by adhesive, stitching, loop stitching, or other coupling.

As shown in FIGS. 6C-6D, at least a portion of the core 102a of the coil 102 can be surrounded by an inner cover 112 (which can also be referred to as “a first cover”). Here, the core 102a of the coil 102 is the structural part of the coil 102, which can be referred to as the core 102a. The inner cover 112 can have a tubular shape. In some examples, the inner cover 112 can cover an entire length of the core 102a of the coil 102. In some examples, the inner cover 112 covers only selected portion(s) of the core 102a of the coil 102. Notably, FIGS. 6C-6D show the core 102a.

In some examples, the inner cover 112 can be coated on and/or bonded on the core 102a of the coil 102. In some examples, the inner cover 112 can be a cushioned, padded-type layer protecting the core 102a of the coil 102. The inner cover 112 can be constructed of various natural and/or synthetic materials. In one particularly example, the inner cover 112 can include a foam material (e.g., expanded polytetrafluoroethylene (ePTFE)). In some examples, the inner cover 112 is configured to be fixedly attached to the core 102a of the coil 102 (for example, by means of textured surface resistance, suture, glue, thermal bonding, or any other means) so that relative axial movement between the inner cover 112 and the core 102a of the coil 102 is restricted or prohibited. In some examples, one or more portions of the inner cover 112 (e.g., a distal end portion) can be fixedly attached to the core 102a of the coil 102 and one or more other portions of the inner cover (e.g., an intermediate portion and/or a proximal end portion) can be movable relative to the core 102a of the coil 102. In some aspects, the inner cover 112 is coupled with the flap sheet 150 of the guard member 104.

In some examples, as shown in FIG. 6C, the docking device 100 can also include a retention member 114 (which may also be referred to as “a second cover” or “an outer cover”) surrounding at least a portion of the inner cover 112 (and the core 102a of the coil 102). In some examples, retention member 114 can extend over the entire length of the inner cover 112. In the illustrated example, the retention member 114 extends over only a portion of the inner cover 112 so that one or more portions of the inner cover 112 (e.g., the proximal and/or distal end portions) are exposed. In particular examples, a proximal end of the retention member 114 can be positioned proximal to a proximal end of the guard member 104. For example, the proximal end of the retention member 114 can be disposed at or adjacent the ascending portion 110b of the coil 102. In some examples, a distal end of the retention member 114 can be positioned distal to a distal end of the guard member 104. For example, the distal end of the retention member 114 can be positioned adjacent the leading turn 106. In some examples, the retention member 114 can cover the functional turns of the coil 102 in the central region 108. However, the retention member 114 does not cover the guard member 104. In some aspects, the retention member 114 can be coupled to the guard member 104, such as by being coupled with the flap sheet 150. Thus, when the docking device 100 is deployed at the native valve and the prosthetic valve is radially expanded within the docking device 100, the retention member 114 at the central region 108 can frictionally engage the prosthetic heart valve and/or the native leaflet tissue.

The retention member 114 can be formed of various materials configured to engage the native tissue and/or prosthetic heart valve to increase friction therebetween and/or promote tissue ingrown. For example, the retention member can comprise a biocompatible fabric material (e.g., polyethylene terephthalate (PET)). In some examples, the retention member 114 can comprise a braided material. In some examples, the retention member 114 can include a woven material.

In some examples, the guard member 104 can be fixedly attached to the retention member 114 and/or the inner cover 112, for example, via a guard attachment 148, such as sutures, adhesive, and/or any other suitable means for attaching.

In some examples, the guard member 104 can extend along a portion (for example, the atrial portion) of the stabilization turn 110 of the coil 102. In some examples, the guard member 104 can extend along at least a portion of the central region 108 of the coil 102 (for example, a portion of the most proximal turn). In some examples, the guard member 104 can extend along a majority (or even an entirety) of the functional turns in the central region 108. In one example, when the docking device 100 is deployed at a native atrioventricular valve, the guard member 104 does not extend into the ascending portion 110b.

In various examples, the guard member 104 can move between a radially compressed state and a radially expanded state. Specifically, the guard member 104 can include a plurality of arms 122 which can be radially expandable and compressible. In the example depicted in FIGS. 6A-6B, the guard member 104 has four panels 140, including one panel configured as a distal lobe 140d and three proximal panels 140p. In other examples, the guard member 104 can have two, three, four, five, six, seven, eight, nine, or more than 10 panels 140. The panels 140 can extend circumferentially along a portion of the coil 102 of the docking device 100. The terminal panel 140p can be in the form of a distal petal 140d, which may or may not include an arm, a wire frame, or a loop. The distal petal 140d may have an arm 122 in a sleeve, or it can include a wire frame 123 as shown between two flap sheets, which wire frame 123 can be the same material as the scaffold 120.

When the guard member 104 is in the radially compressed state, the panels 140 can be radially compressed against the coil 102 so that the radial profile of the docking device 100 is smaller than a predefined threshold, for example, between 2 mm and 3 mm, inclusive. When the guard member 104 moves from the radially compressed state to the radially expanded state, the panels 140 can extend radially outwardly relative to the coil 102. The guard member 104 can be biased toward the radially expanded state. Thus, the guard member 104 can be retained in the radially compressed state by a dock sleeve of a delivery apparatus, and automatically return to the radially expanded state after the dock sleeve is removed.

As shown herein, the guard member 104 can include a scaffold 120 and a flap 118 substantially enclosing the scaffold 120. The shape of the scaffold 120 can generally define the shape of the guard member 104. For example, the scaffold 120 can include a spine 130 and a plurality of arms 122 connected to the spine 130. The spine 130 defines an inner edge of the guard member 104 and can be attached to the coil 102. Each arm 122 can extend radially outwardly from the spine 130 within a corresponding panel 140.

As described herein, radial expansion of the guard member 104 can help preventing and/or reducing paravalvular leakage (PVL). Specifically, radial expansion of the guard member 104 can form an improved seal around a prosthetic valve deployed within the docking device 100. In some examples, the guard member 104 can be configured to prevent and/or inhibit leakage at the location where the docking device 100 crosses between leaflets of the native valve (for example, at the commissures of the native leaflets). For example, without the guard member 104, the docking device 100 may push the native leaflets apart at the point of crossing the native leaflets and allow for leakage at that point (for example, along the docking device or to its sides). However, the guard member 104 can be configured to expand to cover and/or fill any opening at that point and inhibit leakage along the docking device 100.

In some examples, the inner cover 112 and/or the retention member 114 can have slack. For example, FIG. 6A shows that the inner cover 112 can be axially compressed to have slack 115 before radially expanding a prosthetic valve within the docking device 100. The inner cover 112 can be constructed with a low density ePTFE so that the inner cover 112 can be axially compressed and the resulting slack 115 does not significantly impact the radial profile of the docking device 100. When radially expanding a prosthetic valve within the docking device 100, the docking device 100 may be further radially expanded, which can cause the coil 102 to rotate within the native annulus (also referred to as “clocking”). During the clocking, the slack 115 allows the inner cover 112 to be axially stretched and not rotate together with the coil 102 (that is, the coil 102 may slide axially relative to the inner cover 112). Because the guard member 104 can be fixedly attached to the retention member 114, the slack 115 can also prevent the guard member 104 from rotating and pinning open the native leaflets during the clocking.

In various examples, the guard member 104 can help cover an atrial side of an atrioventricular valve to prevent and/or inhibit blood from leaking through the native leaflets, commissures, and/or around an outside of the prosthetic valve by blocking blood in the atrium from flowing in an atrial to ventricular direction (that is, antegrade blood flow)—other than through the prosthetic valve. Positioning the guard member 104 on the atrial side of the valve can additionally or alternatively help reduce blood in the ventricle from flowing in a ventricular to atrial direction (that is, retrograde blood flow).

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (that is, retrograde blood flow). Positioning the guard member 104 on the ventricular side of the valve can additionally or alternatively help reduce blood in the atrium from flowing in the atrial direction to ventricular direction (that is, antegrade blood flow)—other than through the prosthetic valve.

Additional examples and characteristics of the guard member are described in the section “Exemplary PVL Guards” below.

Additional examples of the docking device and its variants, including various examples of the coil, guard member, inner cover, and other components of the docking device, are described in International Publication No. WO/2020/247907, the entirety of which is incorporated by reference herein.

### Exemplary PVL Guards

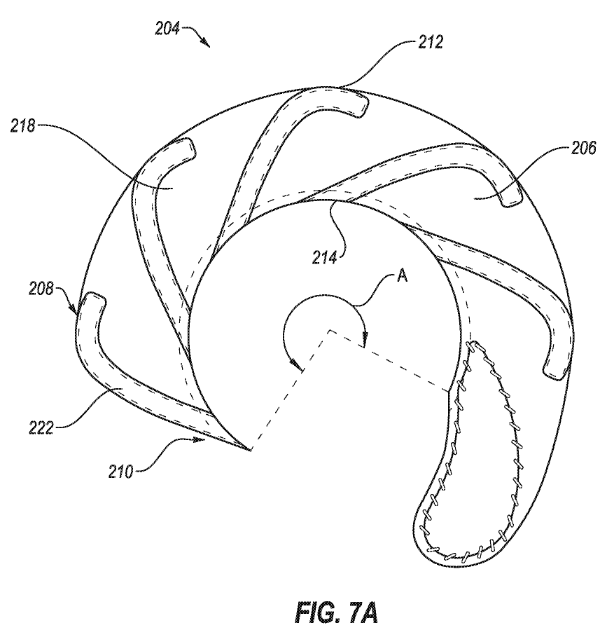

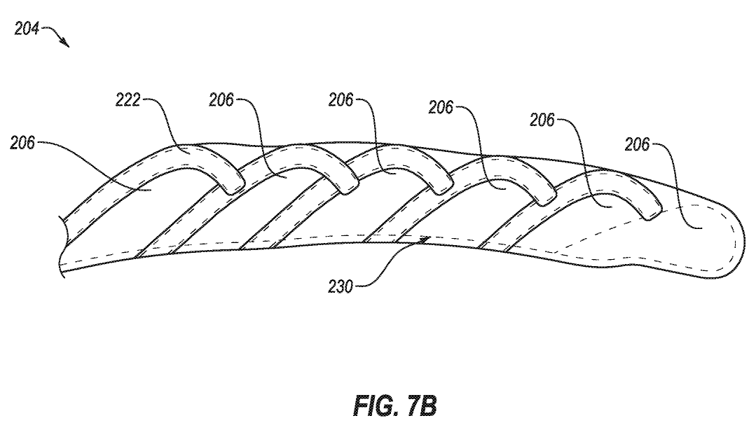

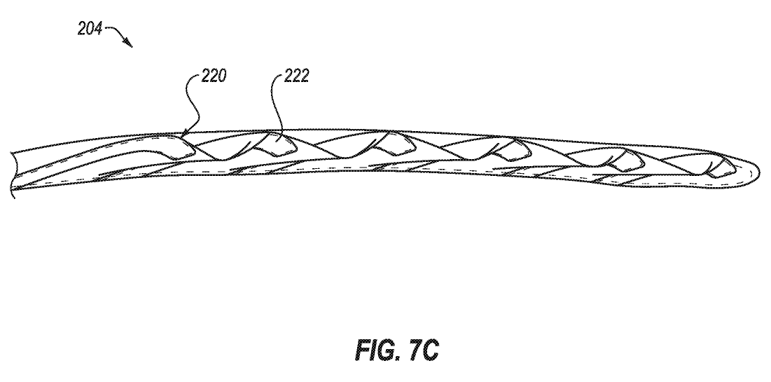

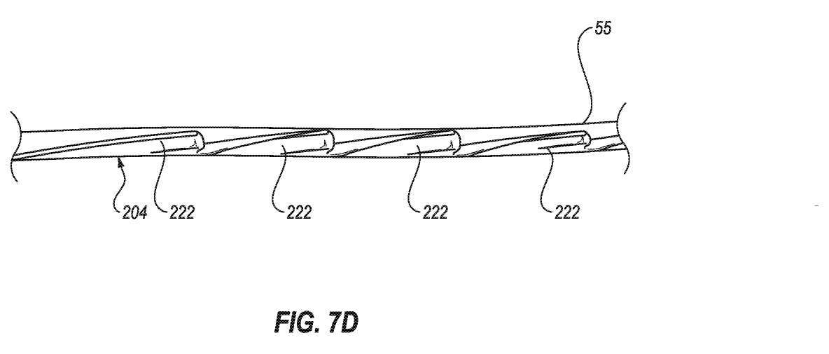

FIGS. 7A-7D depicts an example guard member 204. For a docking device, any of the guard members described herein can be interchangeable. For example, the guard member 204 can replace the guard member 104 of the docking device 100 described above.

The guard member 204 is movable between a radially compresses state (e.g., delivery orientation) and a radially expanded state (e.g., deployed orientation). For example, FIG. 7A shows the guard member 204 in the radially expanded state. FIG. 7B shows the guard member 204 in as being partially collapsed. FIG. 7C show the guard member 204 in the radially compressed state. FIG. 7D shows the guard member in the radially compressed state in a dock sleeve in a delivery orientation that is completely collapsed.

The guard member 204 is also movable between a curved state and a substantially straight state. For example, FIG. 7A shows the guard member 204 in the curved state and FIGS. 7B-7D show the guard member 204 in the substantially straight state.

The guard member 204 is also movable between a folded configuration and an unfolded configuration. For example, FIGS. 7A-7C show the guard member 204 in the unfolded configuration through folded configuration, and FIG. 7D shows the guard member 204 in the folded configuration in the dock sleeve 55 of a delivery apparatus 50 for delivery.

FIG. 7D depicts the guard member 204 in a delivery orientation, for example, when the guard member 204 is retained within a dock sleeve 55 of a delivery apparatus (for example, during delivery of the docking device and after initial deployment of the docking device at the implantation site). In the delivery orientation, the guard member 204 can be folded, radially compressed, and remain substantially straight. FIG. 7A depicts the guard member 204 in a deployed orientation, for example, after the docking device is deployed at the implantation site and the dock sleeve 55 is removed. In the deployed orientation, the guard member 204 can unfold, radially expand, and move to the curved state.

In the example depicted in FIGS. 7A-7D, the guard member 204 includes six panels 206, although the number of panels 206 can be more than six or less than six. The panels 206 can extend circumferentially along a portion of the coil 102. When the guard member 204 is in the radially compressed state (FIGS. 7C-7D), the panels 206 can be radially compressed against the coil 102. When the guard member 204 is in the radially expanded state, the panels 206 can extend radially outwardly relative to the coil 102.

Each panel 206 has an arm 222 with a rounded head portion 208 and a curved elongate base portion 210, which can correspond with the scaffold 120 defined herein. The head portion 208 can be wider than the base portion 210 when the guard member 204 is in the radially expanded state. For each panel 206, the base portion 210 can be attached to the coil 102 (for example, via sutures) and the head portion 208 can extend radially outwardly relative to the base portion 210.

The panels 206 can define an outer edge 212 and an inner edge 214 of the guard member 204. The guard member 204 can be attached to the coil 102 at the inner edge 214.

When the guard member 204 is in the curved state, the inner edge 214 can be curved with an arc angle A. In some examples, the arc angle A is greater than 180 degrees. In some examples, the arc angle A can be between 240 degrees and 360 degrees (for example, about 270 degrees), inclusive.

The overall shape, size, and position of the panels 206 are configured to conform to the native anatomy of the implantation site (for example, the native mitral annulus) and do not puncture or erode through the adjacent native tissue. In some examples, when the guard member 204 is in the radially expanded state, the panels 206 can extend in the same angular direction (for example, clockwise or counterclockwise when viewed from the top or stabilization turn 110 of the docking device). For instance, in FIGS. 7A-7B, all six panels 206 extend in clockwise direction when viewed from above the figures. The panels can be in either the clockwise direction or the counterclockwise direction in different examples.

In some examples, as depicted in FIGS. 7A-7B, the panels 206 have substantially the same size when the guard member 204 is in the radially expanded state. In other examples, at least two of panels 206 can have different sizes when the guard member 204 is in the radially expanded state. In some examples, at least one panel, such as the distal panel can be configured as a petal, with a petal shape.

In some examples, as depicted in FIGS. 7A-7B, the panels 206 have substantially the same shape when the guard member 204 is in the radially expanded state. In other examples, at least two of the panels 206 can have different shapes when the guard member 204 is in the radially expanded state.

When deployed at a native heart valve, the panels 206 can be configured to press against opposing portions of a native heart chamber. For example, when deployed at the mitral valve, the panels 206 can be configured for a surface of the flap 118 to press against an anterior leaflet of the mitral valve and press against a posterior leaflet of the mitral valve, with the perimeter of the flap pressing against the left atrial wall. The arc or rounded shape of the perimeter lip of the flap 218 can be useful for implantation and preventing PVL. In some aspects, the perimeter lip of the flap 218 does not include any indents or scallop shapes, or other features other than being a rounded edge.

The guard member 204 can include a scaffold 220 and a flap 218 substantially enclosing the scaffold 220. The shape of the scaffold 220 generally defines the shape of the guard member 204. For example, the scaffold 220 can include a spine 230 and a plurality of arms 222 connected to the spine 230. The arms 222 can extend radially outwardly from the spine 230. Each arm 222 can extend along a periphery of a corresponding panel 206. The spine 230 can extend along the inner edge 214 of the guard member 204. The spine 230 can be curved to move the guard member 204 to the curved state or straightened to move the guard member 204 to the substantially straight state.

In some examples, the spine 230 and the arms 222 are interconnected to form a unitary piece. For example, the spine 230 and the arms 222 can be laser cut from a single sheet of metal or metal alloy. In other examples, the arms 222 and the spine 230 can be created as separate components and then joined together (for example, via molding, welding, soldering, etc.) to form the scaffold 220.

The scaffold 220 can have the same states or configurations as the guard member 204. For example, the arms 222 can be radially compressed or expanded as the guard member 204 moves between the radially compressed state and radially expanded state. The spine 230 can be curved or straightened as the guard member 204 moves between the curved state and substantially straight state. The spine 230 can also be folded or unfolded as the guard member 204 moves between the folded configuration and unfolded configuration.

Like the scaffold 120, the scaffold 220 can comprise a shape memory material, such as Nitinol. The scaffold 220 can be shape set so that the scaffold 220 is biased toward the deployed orientation. For example, when the guard member 204 is retained within a dock sleeve (for example, the dock sleeve 55) of a delivery apparatus during delivery of the docking device and after initial deployment of the docking device at the implantation site, the arms 222 can be radially compressed and the spine 230 can be substantially straightened and folded. After deploying the docking device and removing the dock sleeve from the guard member 204, the spine 230 can unfold and become curved, and the arms 222 can radially expand under the biasing force.

The flap 218 can be similar to the flap 118. For example, the flap 218 can be configured to be sufficiently elastic so that when the guard member 204 moves from the delivery orientation to the deployed orientation, the flap 218 can accommodate the scaffold 220 (for example, radial expansion of the arms 222 can cause corresponding radial expansion of the flap 218). The flap 218 can also be configured to be atraumatic to native tissue and/or promote tissue ingrowth into the flap 218.

FIG. 8A illustrates an example of a docking device 800a having the coil 802 attached to the guard member 804 with the arms 822 oriented in a clockwise direction. The docking device 800 has five arms 822 with five panels 840 and a terminal petal 840a (e.g., a lobe). As shown, the flap 818 has sleeves 821 for the arms 822, where the sleeves 821 are coupled with the flap sheet 850. In some examples, the flap sheet 850 is folded over the ends of the arms to provide increased protection to native tissue. Also, the terminal petal 840a includes a petal member 841 coupled with the flap sheet 850 to form the flap 118, with stitching (e.g., sutures) forming the lobe shape of the petal member 841.

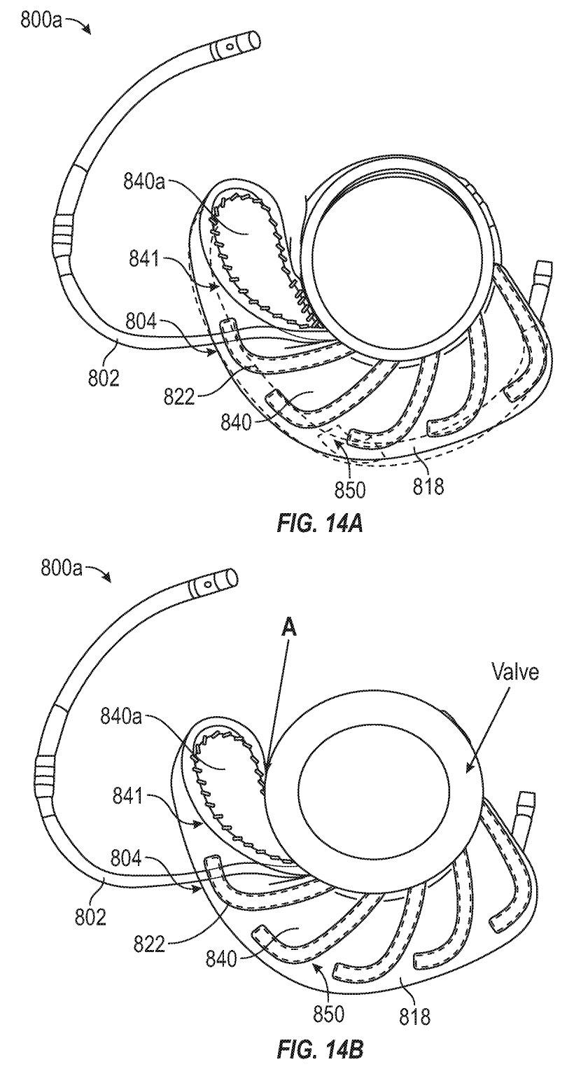

In this example, the terminal petal 840a may include a coil-facing edge that is not connected to, and is spaced apart from, the coil, as shown for example in FIG. 14A. The terminal petal 840a may extend past the atrial turn of the coil to provide increased coverage. Further, referring as well to FIG. 14B, terminal pedal 840a may be curved inwardly in such a way that when the dock is deployed and the prosthetic valve deployed therein, the terminal pedal will contact and/or conform to the outer surface of the valve and provide improved paravalvular sealing, as illustrated by arrow A. The terminal petal 840a may include stitching or a stitch pattern, with the stitching on the inside of the scaffold member as shown in FIG. 14B, to allow the arms 822 to elongate when compressed into the catheter delivery lumen, while the outside is not stitched so that it can expand.

FIG. 8B illustrates an example of a docking device 800b having the coil 802 attached to the guard member 804 with the arms 822 oriented in a clockwise direction. The docking device 800 has eight arms 822 with eight panels 840, where the terminal panel is similar to the other panels (e.g., not a petal). As shown, the flap 818 omits sleeves for the arms 822, where the arms 822 are coupled with the flap sheet 850 via stitching (e.g., sutures), which stitch the arms 822 to the flap 818 by stitching on both sides thereof. Here, the flap sheet 850 can be one, two, or more sheets, where the sheets can be coupled together to form a cavity that receives the arms 822.

FIG. 9A illustrates an example of a scaffold 920 for a guard member. As shown, the scaffold 920 includes arms 922 oriented clockwise, but could be counter clockwise. The scaffold 920 is shown to have six arms 922 and a terminal lobe 923. The arms 922 have a proximal base portion 922b with an elongate proximal region 924 that turns into a medial curved region 925 with a distal head 922h. The distal head 922h is shown to have an aperture 926 formed therein. However, the distal head 922h may be solid and omit the aperture. The aperture 926 can be favorable to provide for less material and weight of the scaffold 920 while still providing a rounded end to inhibit damaging the cover material of the guard member.

FIG. 9B illustrates an example of a scaffold 920 for a guard member. As shown, the scaffold 920 includes arms 922 oriented clockwise, but could be counter clockwise. The scaffold 920 is shown to have five arms 922 and a terminal lobe 923. The five arms 922 of FIG. 9B are thicker in width than the six arms 922 of FIG. 9A, which provides for different mechanical properties for different examples. The wider arms 922 have a proximal base portion 922b with an elongate proximal region 924 that turns into a medial curved region 925 with a distal head 922h. The distal head 922h is shown to have an aperture 926 formed therein. However, the distal head 922h may be solid and omit the aperture. The aperture 926 can be favorable to provide for less material and weight of the scaffold 820 while still providing a rounded end to inhibit damaging the cover material of the guard member. For example, the five arms 922 of FIG. 9B can have width of 0.30 mm, where the six arms 922 of FIG. 9A can have a width of 0.15 mm. Other examples can include widths from about 0.30 mm to about 0.15 mm or other ranges as recited herein. The thickness of each arm 922 in any example can be from about 0.20 mm to about 0.50 mm, or about 0.30 mm, to about 0.40 mm, or any range between any of these points.

FIG. 9C illustrates an example of a scaffold 920 for a guard member. As shown, the scaffold 920 includes arms 922 oriented counterclockwise, but could be clockwise. The scaffold 920 is shown to have five arms 922, with one of the arms being terminal on each end. Here, both ends of the scaffold include an arm 922. As such, no terminal petal is included.

FIG. 9D illustrates an example of a scaffold 920 for a guard member. As shown, the scaffold 920 includes arms 922 oriented counterclockwise, but could be clockwise. The scaffold 920 is shown to have seven arms 922, with one of the arms being terminal on each end. Here, both ends of the scaffold include an arm 922. As such, no terminal petal is included. Also, one of the terminal arms is a serpentine curved arm 962, which has a concave curve 964 and a convex curve 966 relative to the terminal position. While only one arm is shown as a serpentine curved arm 962, any arm can be configured as a serpentine curved arm 962.

FIG. 9E illustrates an example of a scaffold 920 for a guard member. As shown, the scaffold 920 includes arms 922 oriented clockwise, but could be counter-clockwise. The scaffold 920 is shown to have eight arms 922, with one of the arms being terminal on each end. Here, both ends of the scaffold include an arm 922. As such, no terminal petal is included. The arms 922 omit a head and instead have the medial curved region 925 extend to a terminal end 927.

FIG. 9F illustrates an example of a scaffold 920 for a guard member. As shown, the scaffold 920 includes arms 922 that are configured as lobes 929, which are oriented counter-clockwise, but could be clockwise. The lobes 929 are formed by an arm 922 having each end coupled with the spine 930. The scaffold 920 is shown to have eight arms 922 forming four lobes 929, with one of the lobes 929 being terminal on each end. Here, both ends of the scaffold include a lobe. As such, both ends may be considered to include a terminal petal as the lobes may also be referred to as petals. The lobes 929 omit a head and instead are curved.

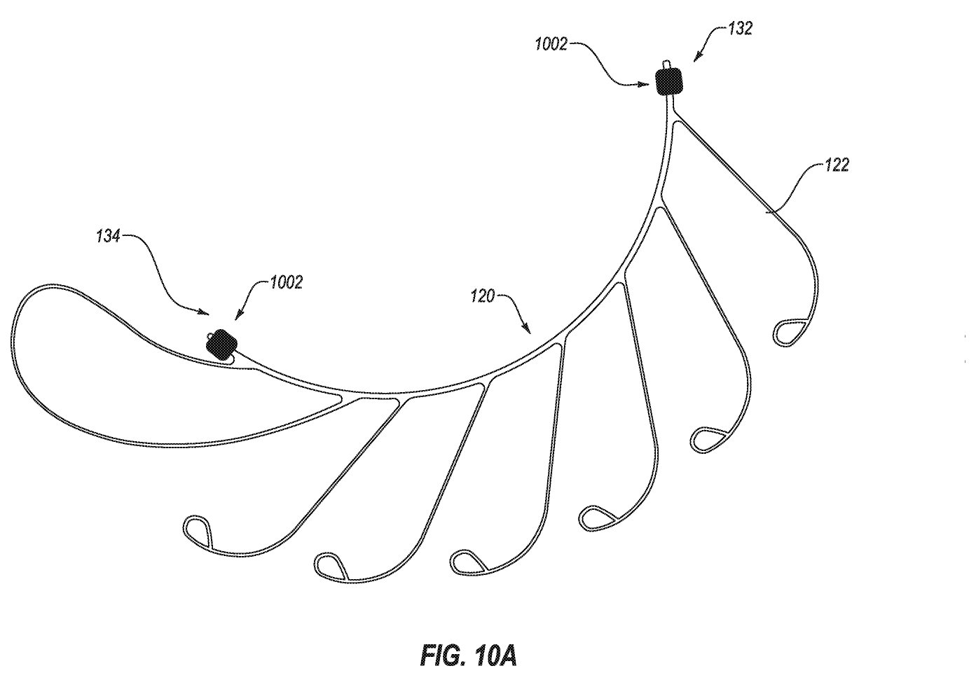

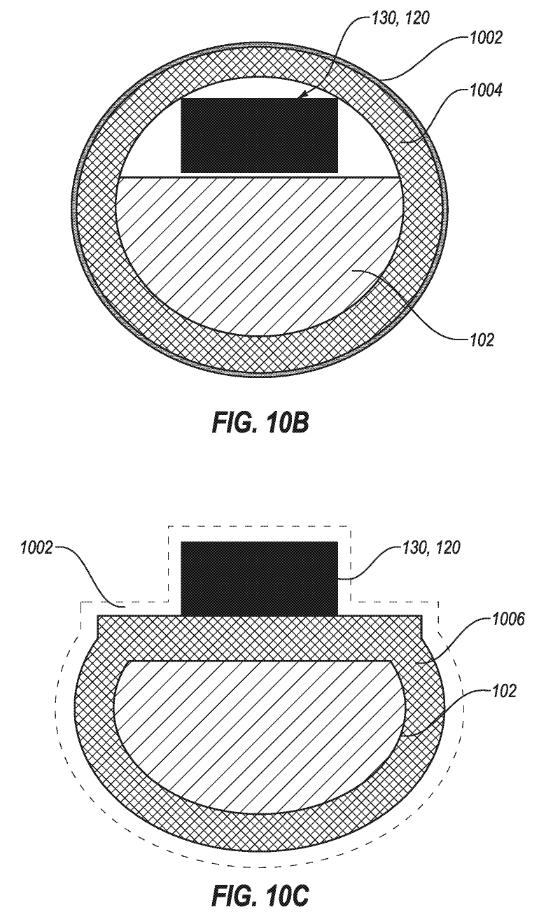

FIG. 10A illustrates an example of a scaffold 120 having a marker 1002 that can be observed with visualization. In some examples, the scaffold 120 can include at least one radiopaque marker 1002 configured to provide visual indication about the location of the guard member 104 relative to its surrounding anatomy, and/or the amount of radial expansion thereof under fluoroscopy. Also, the radiopaque marker 1002 can be used to identify location of the docking device 100 (for example, when a prosthetic valve is subsequently deployed in the docking device 100) under fluoroscopy. For example, one or more radiopaque markers 1002 (e.g., two are shown) can be placed on the scaffold 120. In one particularly example, a radiopaque marker can be disposed at the leading edge 132 and at the trailing edge 134 of the spine 130.

In some examples, the radiopaque marker 1002 can be either replaced with a crimp or can be configured as a crip to illustrate an alternative coupling of the scaffold 120 to the coil 102 of the docking device 100 (e.g., 70). As such, a cover member (e.g., 112, 114) can be formed from a wrapping, which can wrap around the spine 130 of the scaffold 120 and core 102a of the coil 102 so that the arms 122 protrude therefrom. Then, the flap sheet can be installed on the arms. Accordingly, alternative mechanisms of attachment of the scaffold 120 to the coil 102 can be achieved.

FIG. 10B illustrates an example of a cross-sectional profile of the scaffold 120 (e.g., via the spine 130) coupled to the coil 102 with a coating wrap 1004 that is wrapped around the spine 130 and the coil 102 to form a cover. The coating wrap 1004 can be various materials that are biologically compatible, such as expanded polytetrafluorethylene (ePTFE). The coating wrap 1004 can wrap from the leading edge 132 to the trailing edge and then the radiopaque marker 1002 configured as a crimp can be crimped around the ends of the coating wrap 1004 to secure the coating wrap 1004 to the device. This allows the crimp to couple the coating wrap 1004 with the spine 130 and coil 102 together. This configuration can also be shown in FIG. 10A.

FIG. 10C illustrate another example where the spine 130 of the scaffold 120 is outside of the coating 1006 (e.g., small OD ePTFE). Here, the coil 102 is in the coating 1006 with the spine 130 thereon, and the crimp 1008 is formed therearound to secure the spine 130 to the coil 102 so as to be outside the coating. Any number of crimps 1008 can be used, which can be configured and applied as shown with the radiopaque marker 1002 in FIG. 10A. Also, FIG. 10C shows the coil 102 as a dock core that is ground flat on one side to receive the block cross-sectional shaped spine 130.

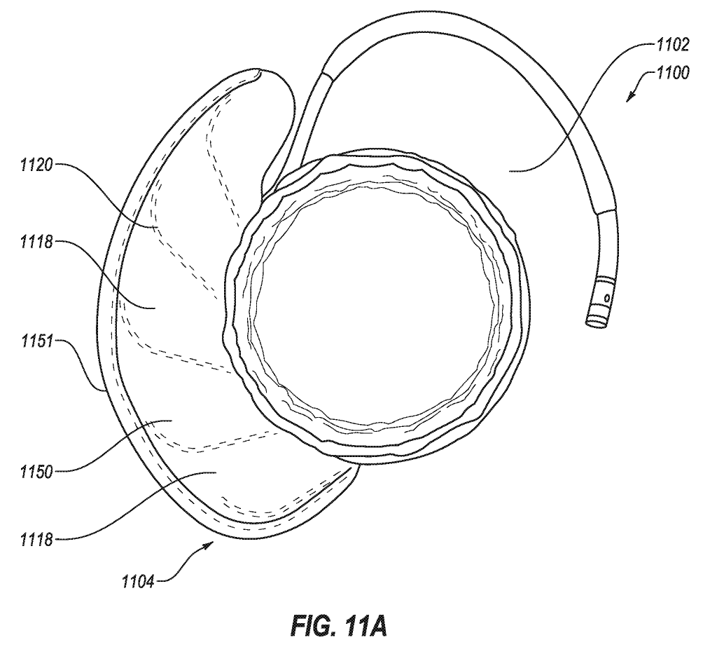

FIG. 11A illustrates an example of a docking station 1100 that includes a coil 1102 with a guard member 1104, which includes a flap 1118 with a flap sheet 1150 over the scaffold 1120. In this example, the flap 1118 includes the flap sheet 1150 having a peripheral lip on a perimeter thereof that includes an edge protector 1151. As such, the edge protector 1151 provides a protective function to the peripheral lip of the flap 1118 and can provide further protection for the arms 1112 and the heads thereof (not shown here). The edge protector 1151 can be a sheet that is folded around the peripheral end of the flap sheet 1150 and stitched thereto. The stitching may be lateral from the head of the arms 1112. Accordingly, the heads or arm ends can be protected from extending past or out of the flap 1118 by the edge protector 1151, which can inhibit the heads or arm ends from poking into the tissue adjacent to the flap 1118 after deployment. The edge protector 1151 can ensure the guard member is atraumatic to the tissue of the left atrium and/or mitral valve anatomy. This can enhance sealing to inhibit PVL. In some aspects, the edge protector 1151 can be a part of the flap material folded over the head of the arms and stitched back to the flap material, which forms the proctor. The flap sheet, such as the bottom sheet can be folded over the tips of the arms, further protecting the anatomy from damage. This may also optimize sealing around the edge of the brim, which is essential for PVL mitigation. Additionally or alternatively, a separate cloth tube or straight piece to be sewn as the edge protector 1151. This adds PVL prevention due to the "3D" nature of the edge with the bulky cloth, mitigation of any tissue damage, but also may improve tissue ingrowth in this area (e.g., enhancing PVL coverage).

The material of the edge protector 1151 can be the same as any type of flap sheet or sleeve described herein. In some examples, the edge protector 1151 can be made from ePTFE, fabric, porous fabric, fuzzy fabric, or any other biocompatible material that can be used as a protective barrier. In some aspects, the material can be selected to promote rapid ingrowth. As such, the material of the edge protector 1151 can include ingrowth hormones, as well as in the material for the rest of the flap 1118.

In some examples, the docking device with the guard member can be configured with a lower profile when in the delivery orientation by reducing the diameter of the coil. That is, the coil core member (e.g., FIGS. 6C-6D 102) can have a reduced diameter to have a lower profile. Also or alternatively, the cover 112 can have a reduced profile by having a reduced diameter. Also or alternatively, the retention member 114 may have a reduced diameter to provide the reduced profile.

As shown in FIG. 11B, a median region 1130 of the coil 1102 can include a thicker diameter region 1132, such as where the coil 1102 loops around the leaflets of the mitral valve. Adjacent thereto, a thinner diameter region 1134 is provided where the guard member 1104 is coupled with the coil 1102. While a gap can be between the thicker diameter region 1132 and the guard member 1104, these components can be adjacent, touching or coupled together. The region of the coil 1102 attached to the guard member 1104 has a reduced dimension to help with folding into the delivery orientation by having the arms 1122 tucked in. As such, the reduced dimension in the thinner diameter region 1134 provides room for the arms 1122 and panels 1140 to fold up. This allows for easier insertion and extraction from the delivery device. For example, the thicker diameter region 1132 can have diameter that ranges from about 1.4 mm to about 2.5 mm, from about 1.5 mm to about 2.2 mm, from about 1.6 mm to about 2.1 mm, or about 1.8 mm to about 2.0 mm, or about 1.9 mm. The thinner diameter region 1134 is always thinner than the thicker diameter region 1132, which can include the thinner diameter region 1134 having a diameter ranging from about 1.1 mm to about 1.6 mm, from about 1.2 mm to about 1.5 mm, from about 1.3 mm to about 1.4 mm. Accordingly, this configuration can be fit into the delivery device with easier installation, and less force is used for deployment of the guard member so that a better installation result can be achieved. The thinner diameter region 1134 can be a smaller covering material, such as a smaller ePTFE material.

Additionally, FIG. 11B illustrates a junction 1137 between the thicker diameter region 1132 and the thinner diameter region 1134. This junction 1137 can include a coupling member 1139 or coupling adhesive that couples the thicker diameter region 1132 to the thinner diameter region 1134. The coupling member 1139 can also include suture material that stiches the thicker diameter region 1132 and the thinner diameter region 1134 together.

In some examples, different techniques can be used to prepare the docking device having the guard member. An exemplary method of making the docking device having the guard member is explained herein; however, variations can be made to achieve the examples illustrated and described herein. In the example, the docking device having the guard member is shown herein (figures) for illustration purposes to show the product of the manufacturing procedure. Although it should be understood that similar or different methods can be used to make docking devices having different guard members by following the patterns and components thereof.

In some examples, the scaffold of the guard member can be obtained by cutting a substrate to form the spine and the arms. Each arm has a head portion and a base portion formed thereon. The base portions are connected to the spine of the scaffold. The head portions are positioned farther away from the spine than the base portions. Each arm can have a tapered shape such that the region of the arm by the head portion is narrower than the base portion. In one specific example, the scaffold can be made by laser cutting a Nitinol sheet. In other examples, the arms and the spine can be created as separate components and then joined together (for example, via molding, welding, soldering, adhesive, etc.) to form the scaffold.

The guard member can be created by enclosing the scaffold into the flap using sleeves in the flap for each arm or lobe. An example method of making the flap can include a base fabric, and then a sleeve fabric can be formed onto the flap to form each sleeve.

In some examples, the scaffold can be connected to the flap via a plurality of sutures stitched therewith. The sutures and stitching can run through specific patterns (e.g., loop stitches) so as to retain the scaffold while also allowing certain movability of the scaffold within the flap.

For example, at least some of the sutures, referred to as loop suture or cross-sutures, can extend across one or more wire segments of the spine or around the spine located at base portions of the lobes of the wireframe. Such cross-sutures can retain each arm within its corresponding sleeve and thereby panel, and restrict lateral movement of the scaffold within the flap.

Additionally, at least some of the sutures, referred to as inner sutures, can extend along and located inwardly of one or more wire segments located at head portions of the arms of the scaffold. As a results, pockets or sleeves can be created between the line of inner sutures and the outer edge of the flap. The pockets or sleeves allow limited sliding movement of the arms within the flap. For example, the head portions of the arms can slide within the pockets or sleeves, thereby allowing the scaffold to move between the radially compressed state and radially expanded state, and/ or between the substantially straight state and curved state. Notably, the cross-sutures do not hinder sliding movement of the arms within the sleeves of the flap.

In some examples, the guard member can be attached to the coil of the docking device. For example, the flap and/or the scaffold of the guard member can be attached to the coil via one or more sutures, loop stiches, wraps, or other fastening feature. The spine and core may be directly bonded together (e.g., adhesive, welding, brazing, etc.), or placed adjacent and wrapped together with a wrapping cover (e.g., FIG. 10B).

Before implanting the docking device, the guard member can be retained within a dock sleeve (for example, the dock sleeve 55). The guard member retained within the dock sleeve can remain in a radially compressed state. For example, the panels can be radially compressed so that they extend along and are substantially parallel to the coil.

### Exemplary Docking Devices

FIG. 14A shows a top view of an example of a docking device with a guard member, which is shown without the valve device. FIG. 14B shows a top view of the example of the docking device with a guard member of FIG. 14A, which is shown with the valve device. These are described in more detail elsewhere in the application.

FIG. 15 shows a plane view of a scaffold 1500 having six arms 1504 where each of the arms tapers/narrows from a wider base 1506 to a narrower end region, e.g. neck 1508, adjacent to head 1510. For example, the arms 1504 may vary in width with a greater width at base 1506 tapering to narrow neck 1508. For example, at least one of the arms, for example, two, three or more of the arms, for example all of the arms, may have a tapering width. For example, at least one of the arms has a wider base width of about 0.2 mm to about 1.0 mm, and a narrower neck width of about 0.1 mm to about 0.5 mm. In one example, at least one of the arms, for example, all of the arms, has a base width of 0.25 and a neck width of 0.12 mm.

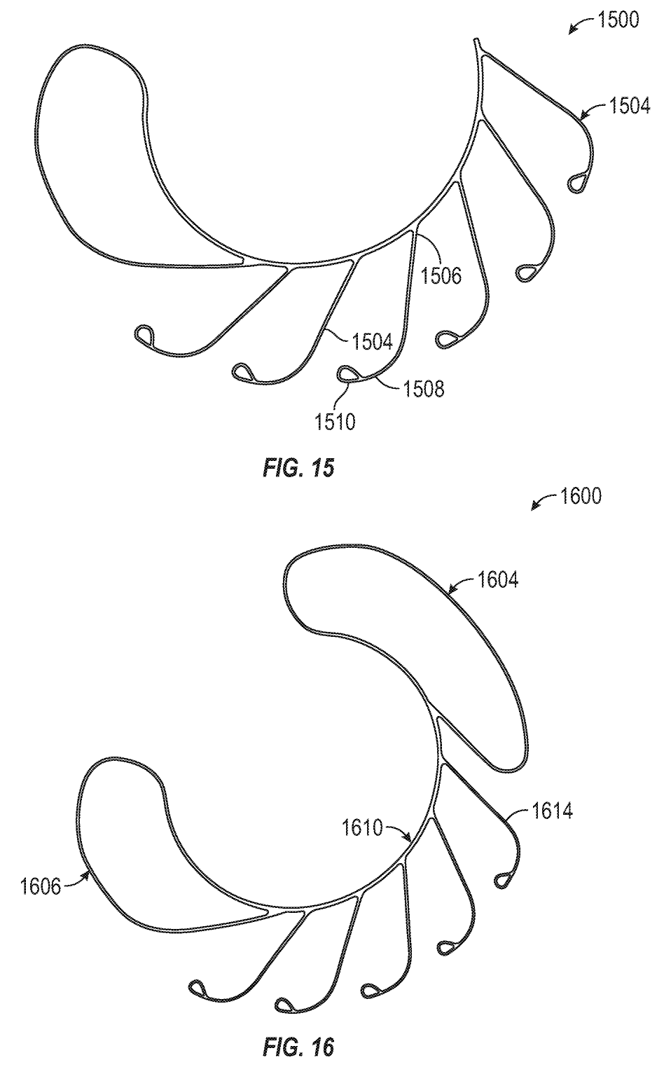

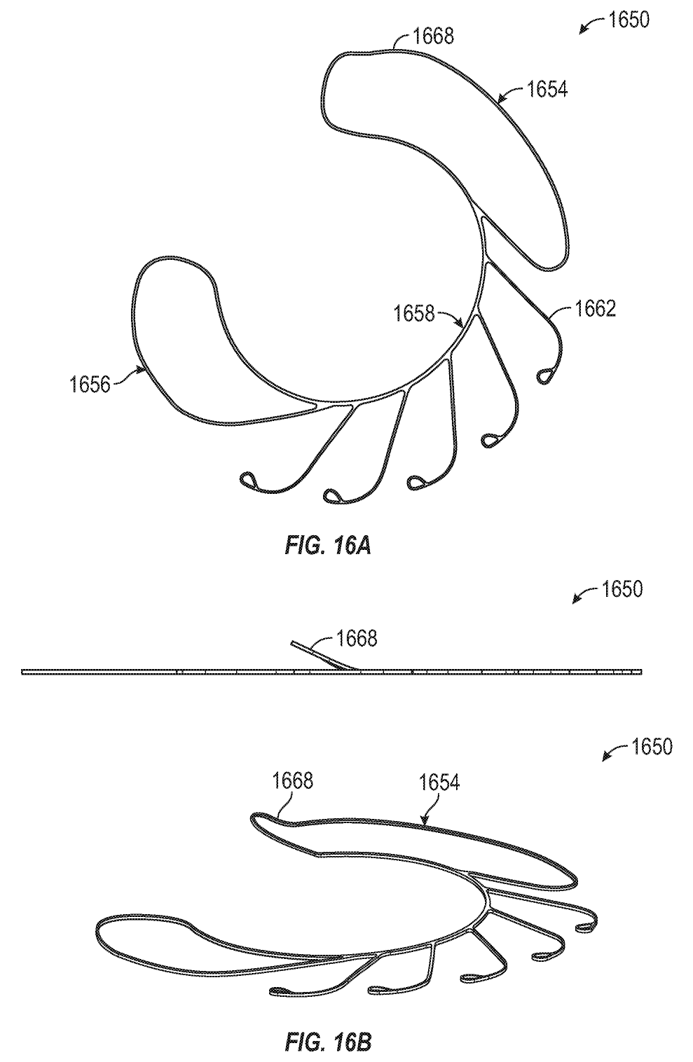

FIGS. 16 and 16A shows alternative examples of a scaffold. Scaffold 1600 shown in FIG. 16 includes two terminal lobes 1604, 1606. Scaffold 1600 is generally flat and/or planar. For example, spine 1610, arms 1614 and lobes 1604, 1606, all define a plane, or lie in a plane, and do not generally extend outside of said plane.

Scaffold 1650 shown in FIG. 16A also includes two terminal lobes 1654 and 1656. Unlike scaffold 1600, scaffold 1650 is not entirely flat or planar. For example, scaffold includes spine 1658 and arms 1662 defining a plane, at least one region, for example, a portion of distal lobe 1654 angled out of the plane defined by spine and arms. For example, distal lobe 1654 may include portion 1668 bent or angled out of the plane to form a scooped tip (e.g., “ski tip”). This feature may be better understood with reference to FIG. 16B. FIG. 16B shows a perspective view and a side view of scaffold 1650 including bent portion 1668 of lobe 1654, to be out of plane with the rest of the scaffold. Bent portion 1668 forms the scooped tip, or “ski tip” structure.

In some examples, not shown, one or both of the terminal lobes 1654, 1656, for example, terminal lobe 1654, may be shape set to fall entirely outside the plane defined by spine and arms. For example, alternative to, or in addition to, “ski tip” portion described with regard to FIGS. 16-16B, the entire terminal lobe 1654 may exist outside said plane, to define an angle of between 5 degrees to 45 degrees. The shape set angled lobe may enable or improve conformation with the anatomy of the patient throughout implant deployment. In some examples, both of the terminal lobes are shape set downward or upward relative to the coil.

It is to be appreciated that each of scaffolds 1500, 1600, 1650 can be fitted with a flap, not shown, as described and shown elsewhere herein, such flap being sized or adapted to fit over the scaffold to form a guard member having one or more of the advantages and features described and shown elsewhere herein.

Turning back to FIGS. 14A and 14B, when the scaffold is in the flap to form a docking device with a guard member with two terminal lobes that are not coupled with the coil, such that the terminal lobes can move relative to the coil. That is, FIGS. 14A and 14B can be modified so that there is a terminal lobe/petal at both ends that is not connected to the coil. As such, when the valve is in the docking device of FIGS. 16A-16B, the valve expands the coil diameter and pushes against the non-attached terminal lobes to compress the valve against the terminal lobes. Accordingly, the compression of the terminal lobe shown in FIG. 14B can be applied to both terminal lobes of the scaffolds of FIGS. 16A-16B.

In some examples, implanting a prosthetic valve at a native annulus involves a two-step implementation procedure. As described above, this procedure includes first implanting a docking device, followed by the deployment of the prosthetic valve within the docking device. In the transient stage of the procedure (following docking device implant and before the prosthetic valve has been implanted), reducing or eliminating movement of the docking device relative to the native anatomy is advantageous. It is also advantageous to improve safety and ease of deployment of the docking device. The docking devices described below may enable increased ease of deployment and maintain positioning relative to the native anatomy. The docking devices described below may simplify the design and reduce implant materials that are not chronically useful (i.e. materials that are not useful beyond the initial implantation procedure). The atrial most functional turn of these designs may have the advantage of increasing attachment surface for the brim feature, therefore increasing docking device stability in the anatomy.

As used herein, the turns of the coil are referred to as defining a lumen diameter and/or as being disposed in a plane that is normal to the longitudinal axis 1701. Because the turns of the coil form a spiral, they do not necessarily define a traditional diameter or plane. The plane referred to herein should be understood to be a plane that bisects the referenced turn of the coil in at a midpoint along the longitudinal axis. In other words, a given turn of the coil will be half above and half below the plane that it is said to be disposed in. A diameter of a given turn of the coil as referred to herein lies in a plane as described above. As mentioned above, the term “diameter” as used in this disclosure does not require that a turn be a complete or perfectly shaped circle but is generally used to refer to a greatest width across opposing points of the turn.

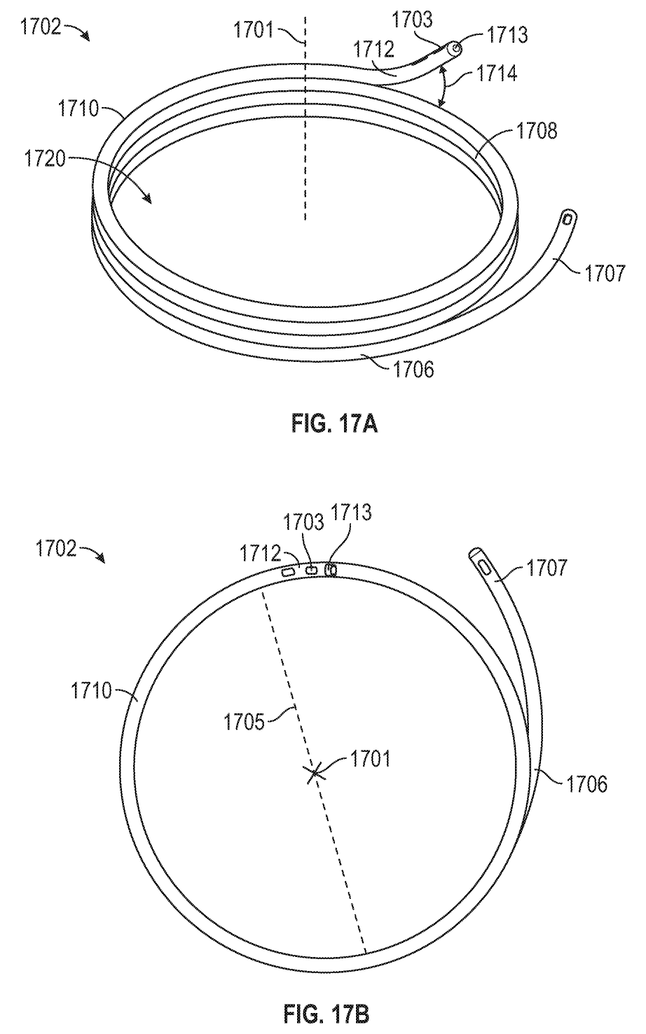

Depicted in FIG. 17A-17B is a coil 1702 shown in the deployed configuration. The coil 1702 can include a stabilization turn 1710 (also referred to as a “stabilization coil” or an “atrial most functional turn” or a “first coil region”), a central region 1708 (also referred to as “functional turns” or a “second coil region”), and a leading turn 1706 (or “leading coil”), each of which are disposed around a longitudinal axis 1701 which extends through a central lumen 1720 of the coil 1702. The central region 1708 can comprise one or more helical turns having substantially equal lumen diameters. The leading turn 1706 can extend from a distal end of the central region 1708 and, in some examples, can have a lumen diameter substantially equal to the lumen diameter of the central region 1708. In some examples, the leading turn can comprise a distal end portion 1707 that extends radially outward. In some examples, the stabilization turn 1710 can extend from a proximal end of the central region 1708 and can have a lumen diameter substantially equal to the lumen diameter of the central region 1708. In some examples, as will be discussed below, the lumen diameter of the stabilization turn can be different than the lumen diameter of the central region 1708.

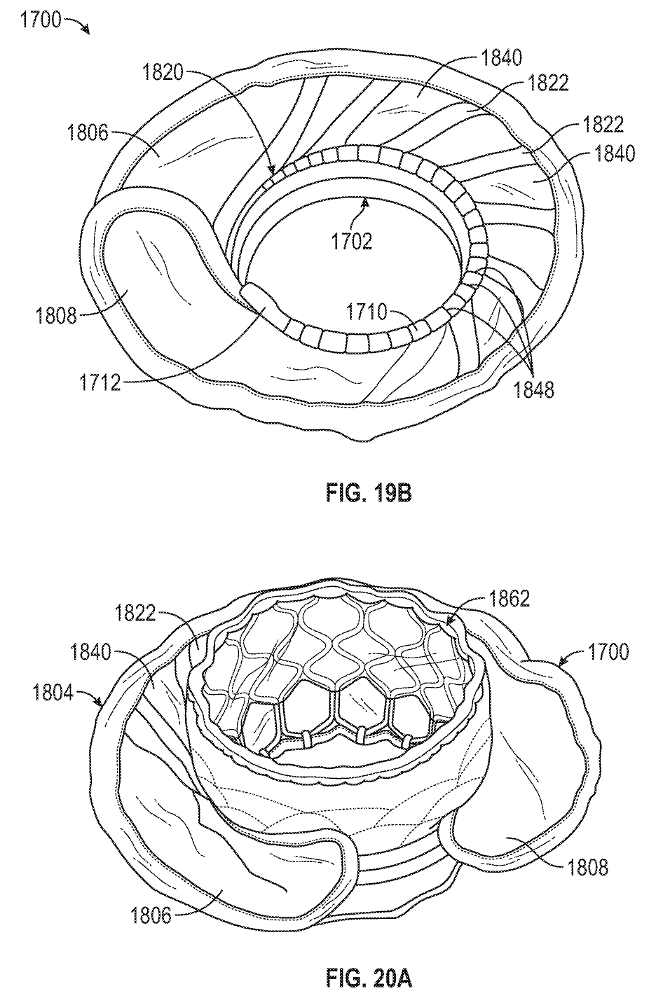

The coil 1702 can be used as a part of a docking device, for example docking device 1700 (FIG. 19A). In some examples, when implanting the docking device at the native mitral valve location, functional turns in a central region 1708 can be disposed substantially in the left ventricle and the stabilization turn 1710 can be disposed substantially in the left atrium. The stabilization turn 1710 can be configured to provide increased stability and improved sealing. In some examples, the points of contact between a docking device comprising the coil 1702 and the left atrial wall can form a plane that is approximately parallel to a plane of the native mitral valve. In some examples, a guard member can be used with the stabilization turn 1710 and configured to provide increased stability to the docking device as it is positioned in the mitral valve.

In some examples, when implanting the coil 1702 at the native mitral valve location, the functional turns in the central region 1708 can be disposed substantially in the left ventricle and the stabilization turn 1710 can be disposed substantially in the left atrium. The stabilization turn 1710 can be configured to provide one or more points or regions of contact between the coil 1702 and the left atrial wall adjacent to the mitral valve, such as at least three points of contact in the left atrium or complete contact on mitral anulus. In some examples, the points of contact between the coil 1702 and the left atrial wall can form a plane that is approximately parallel to a plane of the native mitral valve. In some examples, the contact between the stabilization turn 1710 and the atrial wall can be through an intermediary, such as a guard member.

As seen in the depicted example, the coil 1702 is similar to the coil 102 depicted in FIGS. 6A-6B; however, the coil 1702 omits the ascending portion 110b and the raised stabilization portion 110a. Omitting these portions may reduce required materials, eliminate procedural steps, and/or make deployment easier. Omitting the ascending portion 110b and the raised stabilization portion 110a also may also allow for additional attachment surface for a guard member along the stabilization turn 1710 and can lead to increased stability of the docking device once it is disposed in the anatomy.

In some examples, the central region 1708 can include a plurality of helical turns (for example, the coil 1702 can have three helical turns in the central region 1708). Some of the helical turns in the central region 1708 can be full turns (that is, extending 360 degrees). In some examples, the most proximal turn and/or the most distal turn can be partial turns (for example, extending less than 360 degrees, such as 180 degrees, 270 degrees, etc.). In some examples, the coil 1702 can have more than three helical turns or fewer than three helical turns in the central region 1708.

The lumen 1720 of the central region 1708 can be configured to receive and retain a radially expandable prosthetic valve. The size of the coil 1702 and therefore the lumen diameter 1705 of the lumen 1720 can be generally selected based on the size of the desired prosthetic valve to be implanted into the patient. For example, the lumen diameter of the helical turns in the central region 1708 can be configured to be smaller than an outer diameter of the prosthetic valve when the prosthetic valve is radially expanded so that additional radial force can act between the central region 1708 and the prosthetic valve to hold the prosthetic valve in place.

The stabilization turn 1710 can be configured to help stabilize the coil 1702 in the desired position. In some examples, the radial dimension of the stabilization turn 1710 can be substantially the same as the radial dimension of the coil in the central region 1708. In some examples, as will be described below, the diameter of stabilization turn 1710 is desirably larger than the native annulus, native valve plane, and/or native chamber for better stabilization. In some examples, the stabilization turn 1710 can be a full turn (that is, extending 360 degrees). In some examples, the stabilization turn 1710 can be a partial turn. In some examples the partial turn can extend in a range of 90 degrees to 360 degrees. In some examples the partial turn can extend in a range of 180 degrees to 360 degrees.

In the depicted example, an attachment portion 1712 is disposed at a proximal end portion of the stabilization turn 1710 and can be lifted the axial direction. This attachment portion terminates in a proximal end 1713. The upward flare of the proximal end portion 1712 can be define an angle 1714 which is formed with a plane defined by the helical turns in the central region 1708. In some examples, the angle 1714 can comprise and angle greater than 5-degrees but less than 90 degrees. In some examples, the angle 1714 can comprise and angle greater than 15-degrees but less than 70-degrees. In some examples, the angle 1714 can comprise an angle greater than 10 degrees but less than 50-degrees. In some examples, the angle 1714 is 45 degrees. The proximal end 1713 does not extend more than 12 mm in the axially proximal direction from the stabilization turn, that is the proximal end portion does not extend more than 12 mm in an axial direction from a plane defined by the stabilization turn 1710 that is orthogonal to the longitudinal axis and 1701.

The attachment portion 1712 can be configured to releasably couple the coil 1702 to a delivery apparatus (for example, docking device delivery apparatus 50). The upward flare of the proximal end portion 1712 can be advantageous in coupling the coil 1702 to the delivery apparatus, for example by helping to ensure that the attachment portion 1712 is not obstructed (i.e. by the guard member) and helps to ensure easier access to the attachment portion 1712. In some examples, as discussed above, the coil 1702 can be coupled to the delivery apparatus via a release suture that can be configured to be tied to the coil 1702 and cut for removal. In one example, the release suture can be tied to the coil 1702 through one or more eyelets or eyeholes 1703 located at the attachment portion 1712 of the coil 1702. In some examples, the release suture can be tied around a circumferential recess that is located adjacent the attachment portion 1712 of the coil 1702.

As noted above, in some examples the leading turn 1706 can have a diameter substantially equal to the diameter of the central region 1708. In some examples, a distal end portion 1707 of the leading turn 1706 extends radially outward from the rest of the leading turn 1706. The leading turn 1706 can help more easily guide the coil 1702 around and/or through the chordae tendineae and/or adequately around all native leaflets of the native valve (for example, the native mitral valve, tricuspid valve, etc.). For example, once the leading turn 1706 is navigated around the desired native anatomy, the remaining coil (such as the functional turns) of the coil 1702 can also be guided around the same features. In some examples, the leading turn 1706 can be a full turn (that is, extending 360 degrees circumferentially). In some examples, the leading turn 1706 can be a partial turn. In some examples the partial turn can extend in a range of 90 degrees to 360 degrees. In some examples the partial turn can extend in a range of 180 degrees to 360 degrees. In some examples, the distal end portion of the leading turn 1706 be configured to not extend radially outward from the rest of the leading turn 1706. When a prosthetic valve is radially expanded within the central region 1708 of the coil, the functional turns in the central region 1708 can be further radially expanded. As a result, the leading turn 1706 can be pulled in the proximal direction and become a part of the functional turns in the central region 1708.

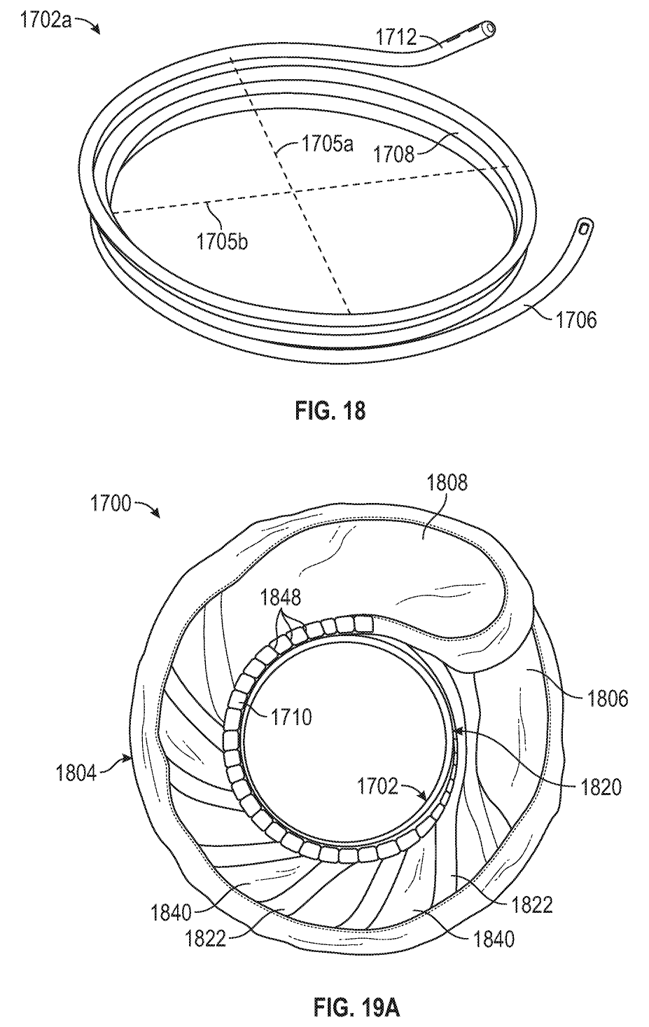

FIG. 18 depicts a coil 1702a which is substantially similar to the coil 1702. Several differences between the coil 1702a and 1702 are described below. The coil 1702a has a stabilization turn 1710a with a first lumen diameter 1705a which can be larger than a second lumen diameter 1705b of the coils in the central region 1708, so that the stabilization turn 1710a can extend radially outwardly so as to abut or push against the walls of the circulatory system, thereby improving the ability of the coil 1702a to stay in its desired position prior to the implantation of the prosthetic valve. This feature may enhancing dock stability (e.g., in larger anatomies). In some examples, the first lumen diameter 1705a is between 5 percent and 50 percent greater than the second lumen diameter 1705b. In some examples, the first lumen diameter 1705a is between 10 percent and 25 percent greater than the second lumen diameter 1705b. In some examples, the first lumen diameter 1705a can be between 25 mm and 30 mm and the second lumen diameter 1705b can be between 20 mm and 25 mm. In some examples, the second lumen diameter 1705b can be 22.7 mm and the first lumen diameter 1705a can be 25 mm. In some examples, the second lumen diameter 1705b can be 22.7 mm and the first lumen diameter 1705a can be 28 mm.

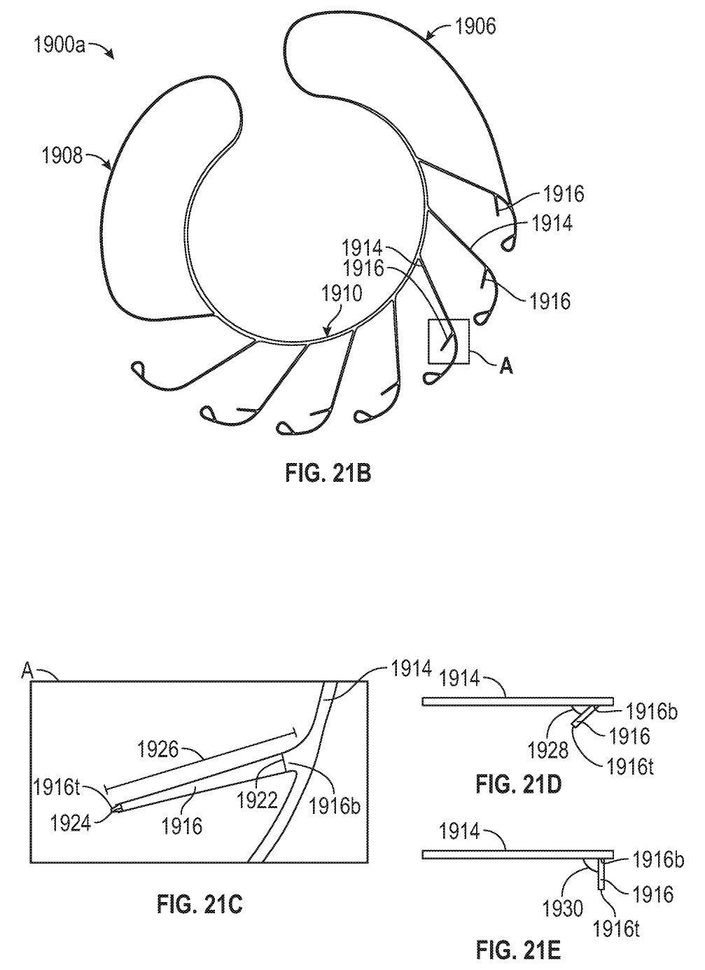

In some examples, a docking device can comprise a coil, such as the coil 1702 in use with a guard member. In some examples, the guard member can comprise a braided sleeve as described in International Publication No. WO2022/087336 which is incorporated by reference herein in its entirety. Additional examples of guard members and other components of the docking device are described in International Application No. WO/2024/37038, which is incorporated by reference herein in its entirety. In some examples, the guard member can comprise any of the guard members described herein. In some examples, the guard member comprises the scaffold 1900 (see FIG. 21A) with two terminal lobes depicted in FIG. 21. In some examples, the guard member can extend along a portion of the stabilization turn 1710 of the coil 1702.

FIGS. 19A-19B depict a docking device 1700 comprising the coil 1702 and a guard member 1804 coupled to the stabilization turn 1710 of the coil 1702. The guard member 1804 can move between a radially compressed state and a radially expanded state. The guard member 1804 can include a plurality of arms defining panels which can be radially expandable and compressible. The guard member can also comprise terminal lobes. In the depicted example, the guard member 1804 has sleeves 1822 with panels 1840 and two terminal lobes, a medial terminal lobe 1806 and a lateral terminal lobe 1808. As shown, the guard member 1804 comprises sleeves 1822 for the arms of the scaffold (such as, for example, arms 1914 of scaffold 1900 depicted in FIG. 21A), where the sleeves 1822 are coupled with the flap sheet. In some examples, the flap sheet is folded over the ends of the arms to provide increased protection to native tissue. Also, the medial terminal lobe 1806 and the lateral terminal lobe 1808 each include a petal member coupled with the flap sheet, with stitching (e.g., sutures) forming the lobe shape of the petal member. As depicted, the medial terminal lobe 1806 and the lateral terminal lobe 1808 can circumferentially overlap.

In some examples, the guard member 1804 is coupled at least partially to an outflow side (the underside as depicted in FIGS. 19A-19B) of the stabilization coil 1710. In some examples, the guard member 1804 is coupled to the outflow side of the stabilization coil 1710 extending circumferentially between 180 and 330 degrees. In some examples, the guard member 1804 is coupled to the outflow side of the stabilization coil 1710 extending circumferentially between 225 and 315 degrees. In some examples, the guard member 1804 is coupled to the outflow side of the stabilization coil extending circumferentially approximately 270 degrees.

In some examples, coil 1702 can comprise a cross-sectional profile similar to that depicted in FIGS. 6C-6D. The guard member 1804 can be fixedly attached to the retention member 114 and/or the inner cover 112, for example, via sutures, adhesive, and/or any other suitable means for attaching. In the depicted example, the guard member 1804 is coupled to the stabilization turn 1710 by a plurality of stiches of suture 1848. In some examples, the guard member can be coupled to the stabilization turn by other means such as thread, stapples, tape, etc.

Attaching the guard member 1804 to the outflow side of the stabilization turn 1710 means that it may rest on top of the native leaflets instead of on top of the stabilization turn 1710 in its final implanted position. This may improve the function of the guard member and its ability to seal to PVL. This may also help to reduce risk of tissue damage (e.g., from suture stitches contacting the native leaflets). When the guard member 1804 is attached to the underside of the stabilization turn, the suture stitches do not touch the native leaflets or any of the patient’s anatomy. Positioning at least a portion of the stabilization turn on top of the guard member may also allow for easier access to the proximal tip 1713 and the attachment portion 1712 of the stabilization turn 1710 for the delivery apparatus.

In some examples, a portion of the guard member may wrap around the stabilization turn 1710 and extend on a proximal side of the stabilization coil. In some examples, the scaffold of the guard member may comprise one or more features which assist the guard member to wrap around the stabilization turn 1710, some of these features are depicted in FIG. 21. In some examples, the portion of the guard member wraps around the stabilization turn 1710 and extends on the proximal side circumferentially between 30 and 135 degrees. In the depicted example, the guard member 1804 axially crosses the stabilization turn 1710 proximal to the medial terminal lobe 1806 and the medial lobe 1808 extends along the proximal side of the stabilization turn 1710. In some examples, this may help to ensure that the medial terminal lobe 1806 of the guard member still maintains positioning above dock core. The medial terminal lobe 1806 of the guard member may be wrapped around the adjacent portion of the stabilization turn 1710, from the outflow side to the inflow side, and secured with suture to the top of the stabilization turn 1710. This means that the functionality of the medial terminal lobe 1806 is not changed with the attachment of a portion of the guard member 1804 on the outflow side of the stabilization turn 1710.

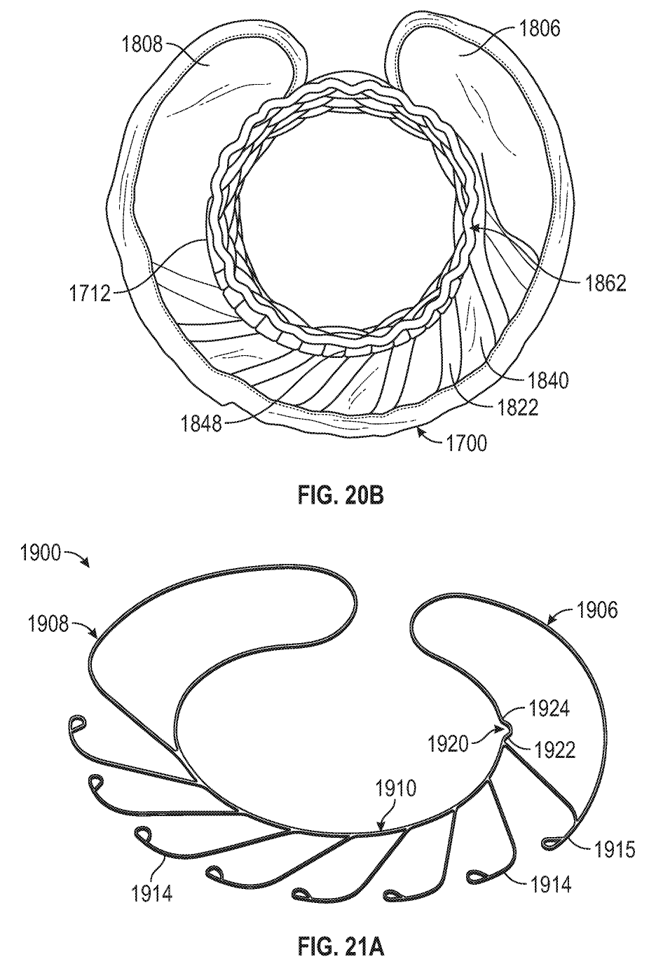

FIGS. 20A-20B depict a coil 1702, a guard member 1804, and a prosthetic valve frame 1862 (the prosthetic valve can be any of the prosthetic valves discussed herein, for example, prosthetic heart valve 62) installed therein. For purposes of illustration, the valve structure (e.g., leaflets) are omitted from FIGS. 20A-20B. The prosthetic valve frame can be configured to be initially inserted into the lumen 1720 of the docking device 1700 in a radially compresses state (e.g., delivery orientation) and then expanded to a radially expanded state. The central region 1708 can be configured to receive and retain a radially expanded prosthetic valve. The size of the coil 1702 and therefore the lumen 1720 can be generally selected based on the size of the desired prosthetic valve to be implanted into the patient. As depicted in FIGS. 20A-20B, the lumen diameter of the helical turns in the central region 1708 can be configured to be smaller than an outer diameter of the prosthetic valve frame 1862 when the prosthetic valve is radially expanded the functional turns in the central region 1708 can be radially expanded. This has the result that radial force can act between the central region 1708 and the prosthetic valve to hold the prosthetic valve frame 1862 in place. As can be seen in FIGS. 20A-20B, with the prosthetic valve frame 1862 expanded within the docking device 1700, the coil expands outwardly such that the terminal lobes 1806, 1808 of the guard member 1804 no longer circumferentially overlap one another.

FIG. 21A shows an example of a scaffold 1900 which comprises a spine 1910, a plurality of arms 1914 and two terminal lobes, a medial terminal lobe 1906 and a lateral terminal lobe 1908. In the depicted example, the plurality of arms 1914 comprises seven arms 1914. In some examples, the medial terminal lobe 1906 can also comprise a radially extending partial arm 1915. Scaffold 1900 is depicted as generally flat and/or planar. For example, spine 1910, arms 1914 and lobes 1906, 1908, as depicted all define a plane, or lie in a plane, and do not generally extend outside of said plane. In some examples, the scaffold 1900 may have some or all of the features described above with respect to other scaffolds described herein, for example the scaffold 120. However, the scaffold 1900 need not include all of the components described above for any other scaffold.

In some examples, the scaffold can be not entirely flat or planar. In some examples, the scaffold is shape set to bring the scaffold to its final form. In some examples, the spine 1910 and arms 1914 may define a plane and at least one region, for example, a portion of one or more of the terminal lobes 1906, 1908 can extend out of the plane defined by the spine 1910 and arms 1914. In some examples, the scaffold 1900 may have a kickout portion 1920 adjacent to the medial lobe 1906. The kickout portion 1920 may have the advantage of more easily wrapping the spine 1910 from the outflow side (bottom as depicted) to the inflow side (top as depicted) of the stabilization turn 1710. In some examples, the kickout portion 1920 can be positioned at location 1820 where the guard member 1804 transitions from the outflow side of the stabilization turn 1710 to the top inflow side of the stabilization turn 1710. In some examples, the kickout portion 1920 can be shape set to be sloped such that it is it is lower on the first side 1922 of the kickout portion 1920 so that it can be attached to the bottom of the stabilization turn 1710, and higher on the second side 1924 where it rests on the top of the stabilization turn 1710.

The scaffold 1900 can be fitted with a flap, not shown in FIG. 21A but as described and shown elsewhere herein to form a guard member. The flap can be sized or adapted to fit over the scaffold. Guard members comprising the scaffold 1900 can have one or more of the advantages and features described and shown elsewhere herein.

FIG. 21B-21E depict an example of a scaffold 1900a which can be similar to the scaffold 1900 except for the differences described below. For example, the scaffold 1900a can comprise a spine 1910, a plurality of arms 1914 and two terminal lobes, a medial terminal lobe 1906 and a lateral terminal lobe 1908. In the depicted example, the plurality of arms comprises six arms. The medial terminal lobe 1906 can also comprise a radially extending partial arm 1915. In some examples, the scaffold 1900a may have some or all of the features described above with respect to other scaffolds described herein, for example the scaffold 1900. For example, though not depicted, the scaffold 1900a may comprise a kickout portion similar to the kickout portion 1920 of scaffold 1900. However, that the scaffold 1900 need not include all of the components described above for any other scaffold.

The scaffold 1900a can comprise one or more retention elements which can engage with heart tissue to help ensure device stability before valve deployment and implant anchoring. In the depicted example, the retention elements comprise tines 1916 which can be coupled to the plurality of arms 1914 and/or the medial terminal lobe 1906. The tines 1916 can extend from a base portion 1916b which is coupled to the arm 1914 or the medial terminal lobe, wherein the base portion 1916b has a first width 1922. The tines 1916 can terminate in a tip portion 1916t which is free and has a second width 1924. In some examples, the first width 1922 is larger than the second width 1924, such that each tine 1916 tapers. In some examples, the tine 1916 tapers to a point. In some examples, the retention elements may be sharp or blunted in order to balance between being atraumatic to the anatomy while still being effective at maintaining implant stability. The tine 1916 may define a length 1926. The retention feature may change in length, width (consistent width along length or tapered width along length), thickness, and tip shape (for example, pointed, rounded, flat, etc.). In some examples, the retention elements are constructed of Nitinol. In some examples, the retention elements are unitarily constructed out of the same sheet of Nitinol as the remainder of the scaffold 1900a.

Scaffold 1900a is depicted as generally flat and/or planar. For example, spine 1910, arms 1914 and lobes 1906, 1908, as depicted all define a plane, or lie in a plane, and do not generally extend outside of the plane defined by the scaffold. In some examples, the scaffold can be not entirely flat or planar. In some examples, as depicted in FIGS. 21D-E, the tines 1916 may extend out of the plane of the scaffold 1900a. In FIG. 21D, the tine 1916 extends at a first angle 1928 relative to the plane defined by the scaffold. In some examples, as depicted in FIG. 21D, the first angle 1928 may comprise a relatively shallow angle. In some examples the first angle 1928 can range from 0 to 90 degrees, from 0 to 45 degrees, from 5 to 45 degrees, and/or from 10 to 30 degrees. In some examples the first angle 1928 is in 10 degrees. In FIG. 21E, the tine 1916 extends at a second angle 1930 relative to the plane defined by the scaffold. In some examples, the second angle 1930 may comprise a relatively large angle. In some examples the second angle 1930 can range from 45 to 180 degrees, from 45 to 135 degrees, and/or from 60 to 90 degrees. In some examples the second angle 1930 is in 90 degrees. The angle between the tine 1916 and the plane defined by the scaffold may determine how atraumatic these features will be and how effective they are at maintaining stability.

The scaffold 1900a can be fitted with a flap, not shown in FIG. 21C but as described and shown elsewhere herein. The flap can be sized or adapted to fit over the scaffold to form a guard member having one or more of the advantages and features described and shown elsewhere herein. In some examples, the tines 1916 can extend through the flap such that the tines 1916 engage directly with the native tissue.

When a prosthetic implant is deployed within the docking device comprising the scaffold 1900a, portions of the docking device and/or guard member may rotate. In some examples, when the prosthetic implant is deployed within the docking device the docking device and or guard member may rotate relative to the native anatomy (e.g., counterclockwise up to 90 degrees). Therefore, the retention elements may move relative to the patient’s anatomy during prosthetic implant deployment. The retention elements may be oriented as to not engage native tissue during this docking device rotation. In some examples, the retention elements may be oriented a clockwise manner as to not cause damage during docking device rotation that is counterclockwise. In some examples, the docking device and/or guard member may rotate clockwise during the deployment of the valve within the docking device and the retention elements may be oriented counterclockwise.

As described above with respect to FIGS. 7A-7D, the guard members described herein can be movable between a radially compresses state (e.g., delivery orientation) and a radially expanded state (e.g., deployed orientation). For example, FIG. 7A shows the guard member 204 in the radially expanded state. FIG. 7B shows the guard member 204 in as being partially collapsed. FIG. 7C show the guard member 204 in the radially compressed state. FIG. 7D shows the guard member in the radially compressed state in a dock sleeve in a delivery orientation that is completely collapsed. Any of the guard members described herein, for example guard a guard member comprising the scaffold 1900 and/or a guard member comprising scaffold 1900a can be similarly movable between a radially compressed (e.g., delivery orientation) and a radially expanded state (e.g., deployed orientation).

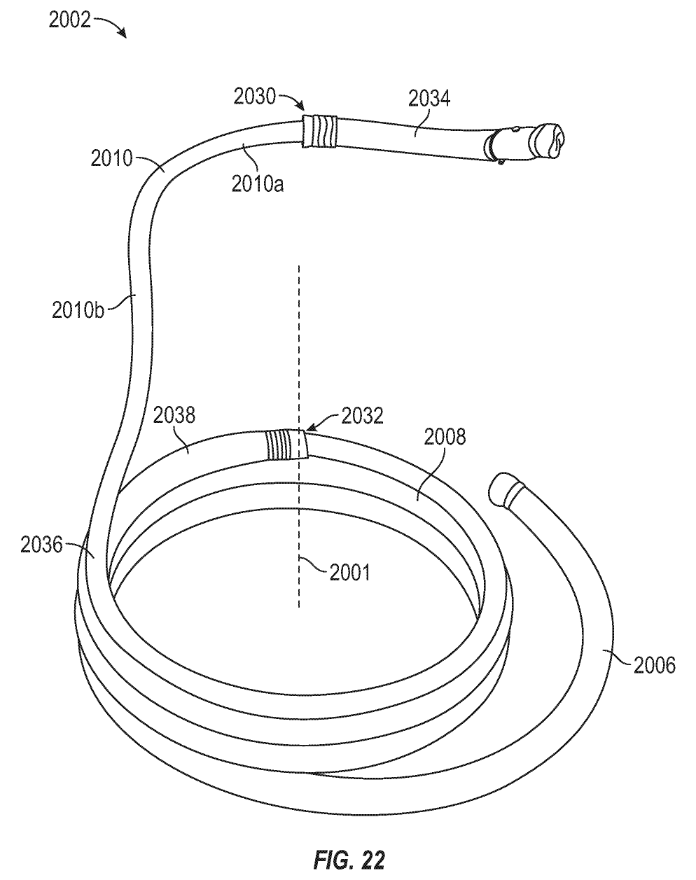

Turning to FIGS. 22-28, in some examples, at least a portion of a coil for a docking device can comprise a cover (such as cover 112 as shown in FIGS. 6C-6D) which at least partially surrounds the core. The cover can, for example, prevent or reduce trauma to native tissue and/or prevent or reduce damage to the delivery device, reduce friction with the native tissue, increase friction with the native tissue and/or prosthetic heart valve, etc. In some examples, the coil can comprise a plurality of covers and/or a plurality of sections of one or more covers, each configured for a particular purpose. For example, a first cover can be provided over all or at least substantially all of the coil, for example, to prevent or reduce trauma to the native tissue. A second cover can extend over a portion of the first cover and can, for example, be configured to increase friction between the cover and native leaflet tissue. In some examples, a plurality of covers of different outer diameters can be used to cover different portions of the coil. Additional information about the covers is provided below and can be found in International Publication No. WO 2022/087336.

In some examples, it is desirable to have a coil diameter that varies over the length of the coil. In some examples, there are two different coil diameters each extending along one or more portions of the length of the coil. In some examples, the docking device with the guard member can be configured with a lower profile when in the delivery orientation by reducing the diameter of a portion of the coil. In some examples, the coil can have a reduced diameter extending over at least the portion of the coil which is adjacent to the position where the guard member is attached. The reduced diameter may result in a lower profile by providing more space for the guard member while in the radially compressed delivery orientation. In some examples, a cover can result in the reduced coil profile (e.g., by comprising a reduced diameter in some portions of the coil).

Different cover diameters along the length of the coil may be provided in several ways. In some examples, a plurality of separate covers each with a different diameter may be attached to the core to result in a smaller outer diameter on portions of the coil while maintaining a larger outer diameter on other portions of the coil.

In some examples, two covers are used such that they partially overlap one another, this can help ensure a smooth transition between the two separate covers. In some examples, at least one cover comprising a smaller outer diameter can be used with at least one other cover having a larger outer diameter. In some examples, the smaller outer diameter cover can be flared radially outward at the junction of the two covers such that the smaller outer diameter cover axially overlaps the larger diameter cover. This is discussed in detail below.

In some examples, a single cover can be attached that provides a larger outer diameter along segments of the coil and a smaller outer diameter in other segments of the coil. In some examples, a single cover can be used on the coil and that cover can be compressed in certain sections to reduce the outer diameter. In some examples, a single cover is used on the coil and the cover can be drawn down in certain sections to reduce the outer diameter. In some examples two or more covers are used such that they overlap one another, with at least one cover having a smaller outer diameter and covering substantially all of the coil and at least one other cover having a larger outer diameter and being disposed radially outward and axially overlapping with the cover with the smaller outer diameter.

Depicted in FIG. 22 is a coil 2002 shown in the deployed configuration which can have similar features to the coils discussed elsewhere herein, including, for example, a leading turn 2006 (or “leading coil”), the central region 2008 (also referred to as a “second coil region”), and the stabilization turn 2010 (also referred to as a “stabilization coil” or a “first coil region”) around a longitudinal axis 2001 which extends through a central lumen of the coil. However, the coil 2002 need not include all of the components described above for any other coil. In some examples, the coil 2002, may omit ascending portion 2010b and the raised stabilization portion 2010a, in other words, the coil 2002 may have features similar to the coil 1702.

As depicted, the coil 2002 can comprise covers of different outer diameters which cover different segments of the coil 2002. These different outer diameters result from cover diameter changes, or transition regions, along the length of the coil 2002. In the depicted example, the coil 2002 comprises two transition regions. These are shown as a first transition region 2030 at the proximal tip portion of the coil and a second transition region 2032 between the central region 2008 and the atrial stabilization turn 2010. In the depicted example, there are three segments, a first segment 2034 which comprises a cover with a first cover diameter, a second segment 2036 which comprises a cover with a second cover diameter, and a third segment 2038 which comprises a cover with a third cover diameter. In some examples, there may be more or fewer transitions regions and more or fewer segments with different cover diameters. In some examples, the coil 2002 comprises a larger diameter cover in the distal ventricular portion of the dock and a smaller diameter cover for the proximal atrial portion of the dock.

The different segments comprising different cover diameters may provide several advantages. In some examples, the first segment 2034 extends along at least a portion of the proximal tip portion of the coil 2002 and comprises a larger diameter. There may be one or more advantages to having the larger coil outer diameter at the proximal tip of the coil such as to maintain implant release efficacy. In some examples, the larger coil outer diameter at the proximal tip of the coil can be useful for the delivery device release mechanism and interactions with the catheter system, for example, the docking device delivery apparatus 50.

In some examples, the second segment 2036 extends along at least a portion of the stabilization turn 2010 of the coil 2002 and comprises a smaller diameter cover. The smaller diameter cover on the stabilization turn 2010, to which the guard member is coupled, can have the advantage of a reduced profile delivery orientation by providing more space for the guard member while it is in the radially compressed delivery orientation. In other words, the reduced dimension in the second segment 2036 provides room for the arms and panels of the guard member to fold up, thus allowing for easier insertion and extraction from the delivery device. Refer to FIGS. 7A-7D and the associated specification for a description of the delivery orientation of the guard member and the transition between a radially compresses state (e.g., delivery orientation) and a radially expanded state (e.g., deployed orientation).

In some examples, the third segment 2038 extends along the central region 2008 of the coil 2002 and comprise a larger diameter cover. This larger diameter cover on the functional turns where the coil 2002 loops around the leaflets of the mitral valve helps to support prosthetic implant anchoring and retention in the anatomy.

In some examples, the first segment 2034 and the third segment 2038 can have diameters that range from 1.4 mm to 2.5 mm, from 1.5 mm to 2.2 mm, from 1.6 mm to 2.1 mm, or 1.8 mm to 2.0 mm, or 1.9 mm. In some examples, the second segment 2036 can have a diameter ranging from 1.1 mm to 1.6 mm, from 1.2 mm to 1.5 mm, from 1.3 mm to 1.4 mm. This configuration can be fit into the delivery device with less force is used for deployment of the guard member so that an easier installation can be achieved.

FIG. 22 also illustrates the first transition region 2030 between the first segment 2034 and the second segment 2036 and the second transition region 2032 between the second segment 2036 and the third segment 2038. The first transition region 2030 and/or the second transition region 2032 can include a coupling member or coupling adhesive that couples the cover of the first segment 2034 to the cover of the second segment 2036 and/or couples the cover of the second segment 2036 to the cover of the third segment 2038. The coupling member can also include suture material that stiches the covers of the different segments together. In some examples, the coupling member can comprise a crimp of a metallic tube (such as a radiopaque marker band). In some examples, the first transition region 2030 and/or the second transition region 2032 can comprise any of any other of the other coupling methods described herein.

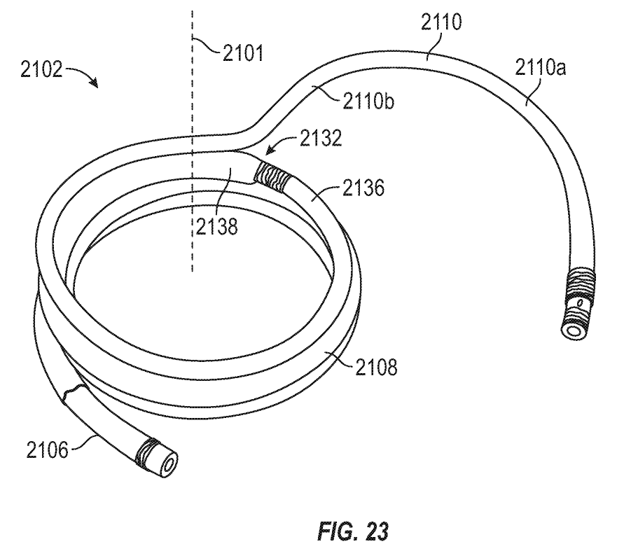

Depicted in FIG. 23 is a coil 2102 as shown in the deployed configuration which can have similar features to the coils discussed elsewhere herein. In some examples, the coil 2102 shares features with the coil 2002. In some examples, the coil 2102 described below is the same as the coil 2002, except for the differences described below. However, the coil described below need not include all the components described above. In some examples the coil 2102 comprises a leading turn 2106 (or “leading coil”), the central region 2108 (also referred to as a “second coil region”), and the stabilization turn 2110 (also referred to as a “stabilization coil” or a “first coil region”) around a longitudinal axis 2101 which extends through a central lumen of the coil 2102. In some examples, the coil 2102, may omit ascending portion 2110b and the raised stabilization portion 2110a, in other words, the coil 2102 may have features similar to the coil 1702.

The coil 2102 may be comprise one or more covers of different diameters which may extend over one or more segments of the coil 2102. In the depicted example, the coil 2102 comprises a transition region which is shown as transition region 2132 between a coil segment with a first diameter and a coil segment with a second diameter. In the depicted example, there are two segments with covers of different diameters, a first segment 2136 which comprises a cover with a first cover diameter, a second segment 2138 which comprises a cover with a second cover diameter.

In some examples, the first segment 2136 extends along at least a portion of the stabilization turn 2110 of the coil 2102 and comprises a smaller diameter cover. The smaller diameter cover on the stabilization turn 2110, to which the guard member is coupled, can result in the benefit of a reduced profile delivery orientation by providing more space for the guard member while it is in the radially compressed delivery orientation. In other words, the reduced dimension in the first segment 2136 provides room for the arms and panels of the guard member to fold up, thus allowing for easier insertion and extraction from the delivery device. Refer to FIGS. 7A-7D and the associated specification for a description of the delivery orientation of the guard member and the transition between a radially compresses state (e.g., delivery orientation) and a radially expanded state (e.g., deployed orientation).

In some examples, the second segment 2138 extends along at least a portion of the central region 2108 of the coil 2102 and comprise a larger diameter cover. This larger diameter cover on the functional turns of the central region 2108 where the coil 2102 loops around the leaflets of the mitral valve helps to support valve anchoring and implant retention in the anatomy.

FIG. 23 also illustrates a transition region 2132 between the first segment 2136 and the second segment 2138. This transition region 2132 can include a coupling member or coupling adhesive that couples the first segment 2136 to the second segment 2138. The coupling member can also include suture material that stiches the first segment 2136 and the second segment 2138 together. In some examples, the coupling member can comprise a crimp of a metallic tube (such as a radiopaque marker band). In some examples, the first segment 2136 and the second segment 2138 can be coupled by any other of the methods described herein.

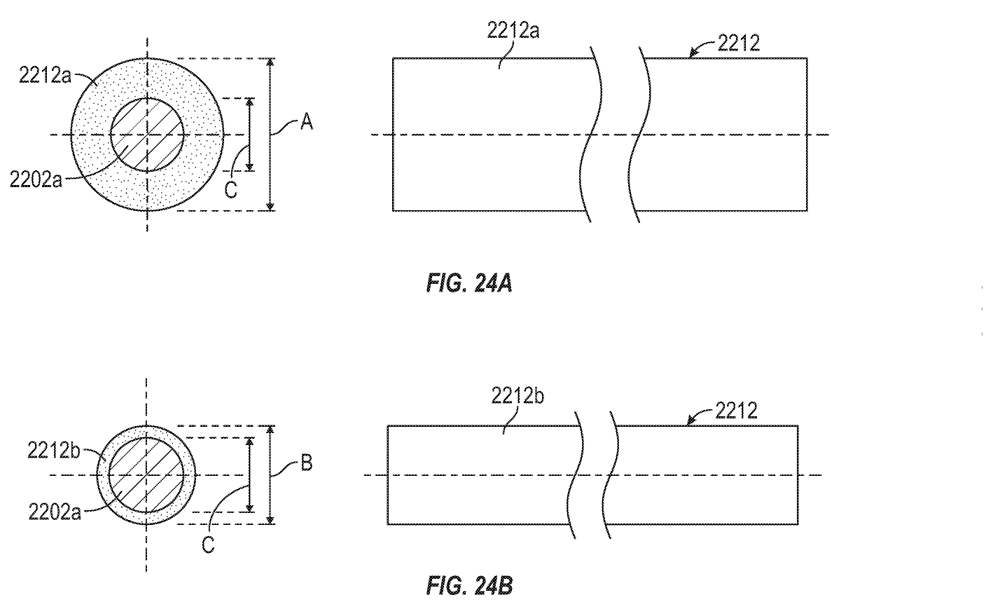

FIGS. 24A-24B depict segments and cross sections of a coil, for example any of the coils described above. The coil can comprise a core 2202a which can be surrounded by a cover 2212 (which can also be referred to as “an inner cover”). Here, the core 2202a of the coil is the structural part of the coil. The cover 2212 can have a tubular shape and can be configured to extend over the core 2202a. In some examples, the cover 2212 can cover an entire length of the core 2202a of the coil. In some examples, the cover 2212 covers only selected portion(s) of the core 2202a of the coil. In some examples, different segments of the cover 2212 can comprise different diameters. The outer diameter of the coil may be the result of the cover 2212 over the core 2202a. In some examples, the cover 2212 can comprise one or more cover segments 2212a with a first diameter A. In some examples, the cover can comprise one or more cover segments 2212b with a second diameter B. In some examples, the diameter of the core 2202a is constant along the length of the coil and defines a coil diameter C. In some examples, the diameter and or the shape of the core of the coil can vary over the length of the coil. In some examples, the diameter of both the core 2202a and the cover 2212 may vary along different portions of the coil.

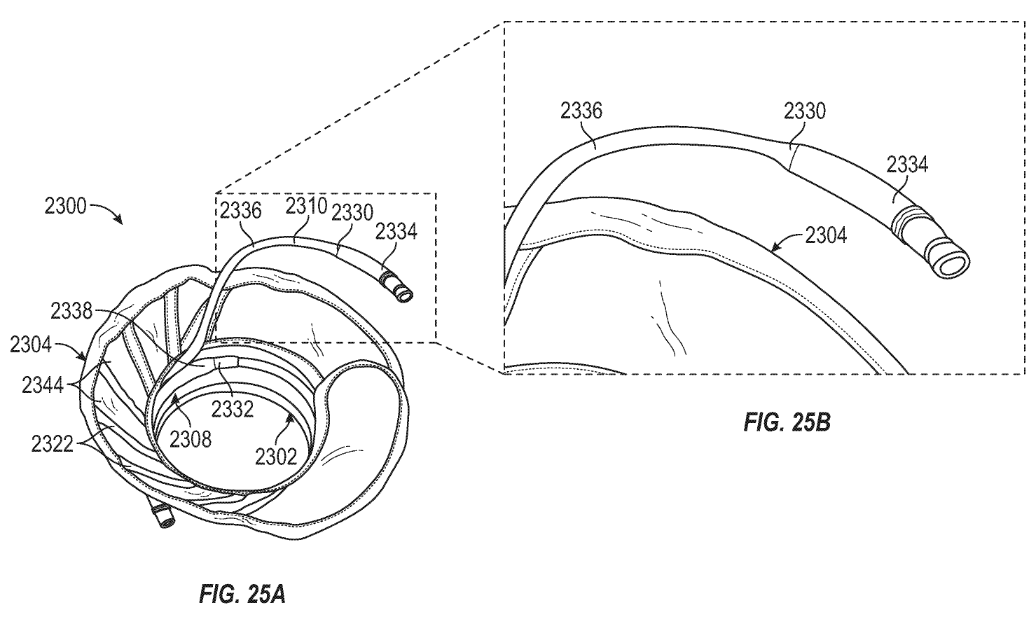

FIGS. 25A-25B depict a docking device 2300 comprising a coil 2302 with a guard member 2304 attached. In some examples, the coil 2302 may have some or all of the features described above with respect to the other coils described herein, for example either the coil 2002 or the coil 2102. In some examples, the guard member 2304 may have some or all of the features described above with respect to the other guard members described herein, for example the guard member 2304 may comprise either the scaffold 1900 or the scaffold 1900a.

The coil 2302 comprises different diameters result from cover diameter changes, or transition regions, along the length of the coil 2302. In the depicted example, the coil 2302 comprises two transition regions. These are shown as a first transition region 2330 at the proximal tip portion of the coil and a second transition region 2332 between the central region 2308 and the atrial stabilization turn 2310. In the depicted example, there are three segments, a first segment 2334 which comprises a cover with a first cover diameter, a second segment 2336 which comprises a cover with a second cover diameter, and a third segment 2338 which comprises a cover with a third cover diameter. In some examples, there may be more or fewer transitions regions and more or fewer segments with different cover diameters. In some examples, the coil 2302 comprises a larger diameter cover in the distal ventricular portion of the dock and a smaller diameter cover for the proximal atrial portion of the dock.

As depicted, the first segment 2334 extends along at least a portion of the proximal tip portion of the coil 2302 and comprises a larger diameter which may be useful for the implant release mechanism and interactions with the catheter system. As depicted, the second segment 2336 extends along at least a portion of the stabilization turn 2310 of the coil 2302 and comprises a smaller diameter cover. The smaller diameter cover on the stabilization turn 2310, to which the guard member 2304 is coupled, can have the advantage of a reduced profile delivery orientation by providing more space for the guard member 2304 while it is in the radially compressed delivery orientation. In other words, the reduced dimension in the second segment 2036 provides room for the arms 2322 and panels 2344 of the guard member 2304 to fold up, thus allowing for easier insertion and extraction from the delivery device. As depicted, the third segment 2338 extends along the central region 2308 of the coil 2302 and comprise a larger diameter cover this may help to support valve anchoring and implant retention in the anatomy.

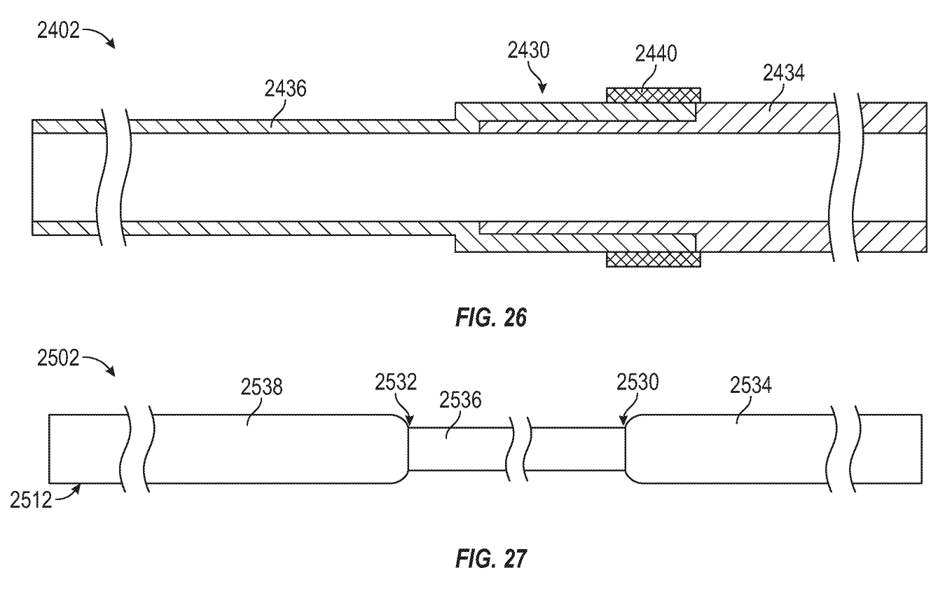

FIG. 26 depicts a portion of a cover assembly 2402 for a coil, comprising a transition region 2430 between a first diameter cover 2434 and a second diameter cover 2436. In the depicted example, the first diameter cover 2434 has a larger diameter than the second diameter cover 2436. The transition region 2430 comprises overlapping the second diameter cover 2436 on top of the first diameter cover 2434. This can be accomplished, for example, by flaring the second diameter cover 2436 radially outward so that it can axially overlap with the first diameter cover 2434, then fitting the first diameter cover 2434 inside of the second diameter cover 2436. The two covers are then secured using a coupling member 2440, coupling adhesive, and/or any other means for coupling the thinner diameter region to the thicker diameter region. The coupling member can also include suture material that stiches the thicker diameter region and the thinner diameter region together. In some examples, the thinner diameter region and the thicker diameter region are coupled together using a lasso stich technique. In some examples, the thinner diameter region and the thicker diameter region are coupled together with a suture wrap technique. This suture wrap technique can minimize penetrations of the cover material. This radial wrapping technique can, for example, reduce or eliminate tearing the cover material at the transition region.

FIG. 27 depicts a coil 2502 which can comprise a single cover 2512 with different diameters along the different segments of a coil. As depicted, there can be one or more transition regions between segments of the cover 2512 with different diameters. In some examples, the cover comprises a larger outer diameter cover along some segments of the coil and a smaller outer diameter cover in other segments of the coil with shoulders at transitions regions between the different diameters. In the depicted example, the coil 2502 comprises a first transition region 2530 between a first diameters cover 2534 and a second diameter cover 2536 and a second transition region 2532 between the second diameters cover 2536 and a third diameter cover 2538. In the depicted example, the transition regions 2530, 2532 comprise a relatively abrupt change of diameter between the first diameters cover 2534 and the second diameter cover 2536 and between the second diameters cover 2536 and the third diameter cover 2538. In some examples, the transition regions 2530, 2532 may comprise a more gradual change in diameter.

In some examples, the cover 2512 can be compressed at desired locations to provide a smaller diameter. In some examples, the cover is an ePTFE tube, and compression results in an increased density and stiffness in the compressed area. In some examples, starting with a cover (before compression) that has a lower density allows the compression to take place. It is important to note that ePTFE density is closely related with durability of the material and the tissue ingrowth properties (for example, high density may improve durability, and low density may improve cellular adhesion and ingrowth).

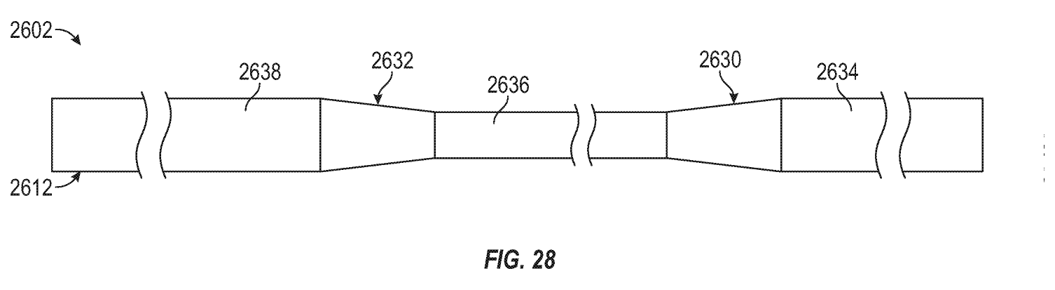

FIG. 28 depicts a coil 2602 which can comprise a single cover 2612 with different diameters along the different segments of the coil 2602. As depicted, there can be one or more transition regions between segments of the cover 2612 with different diameters. In some examples, the cover comprises a larger outer diameter cover along segments of the coil and a smaller outer diameter cover in other segments of the coil with gradual transition regions between the different diameters. In the depicted example, the coil 2602 comprises a first transition region 2630 between a first diameters cover 2634 and a second diameter cover 2636 and a second transition region 2632 between the second diameters cover 2636 and a third diameter cover 2638. In the depicted example, the transition regions 2630, 2632 are sloped.

In some examples, the cover 2612 can be drawn down at desired locations in order to provide the smaller cover diameter. In some examples, the cover is an ePTFE tube and drawing down of the cover can result in a decreased density and stiffness in this area. In some examples, starting with a cover (before drawing down) should be of higher density to allow the drawing down to take place. It is important to note that ePTFE density is closely related with durability of the material and the tissue ingrowth properties (for example, high density may improve durability, and low density may improve cellular adhesion and ingrowth).

As described above, it may be desirable to have a coil diameter that varies over the length of the coil. The coils described herein with different diameters may have the advantage of a lower profile when in the delivery orientation by reducing the diameter of a portion of the coil while maintaining a larger diameter cover on the functional turns where the coil loops around the leaflets of the mitral valve helps to support prosthetic implant anchoring and retention in the anatomy. In some examples, a larger coil outer diameter at the proximal tip of the coil can be useful for the delivery device release mechanism and interactions with the catheter system.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Implantation

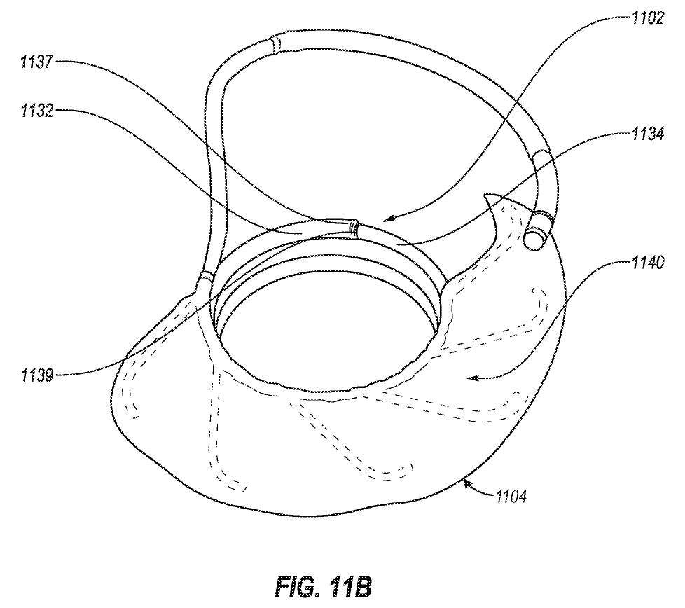

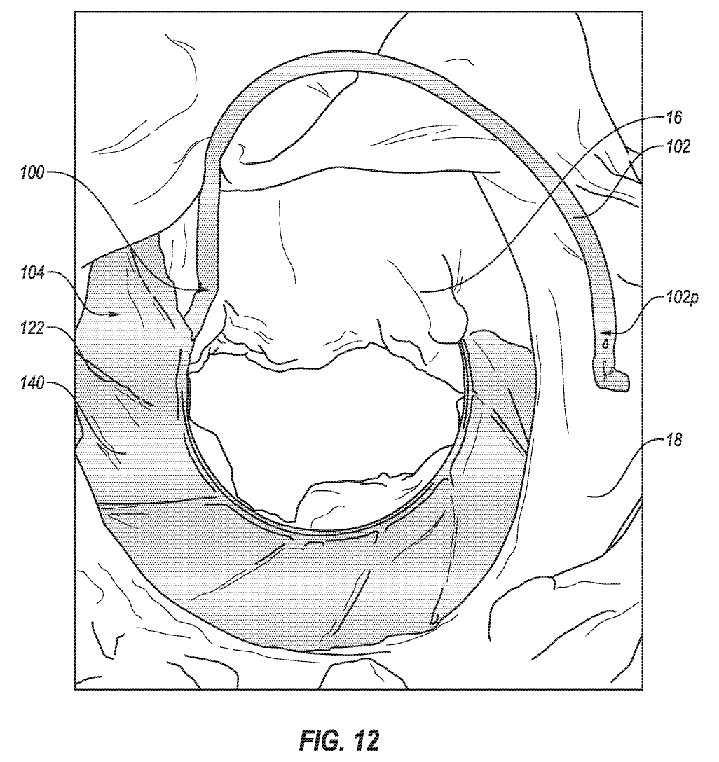

FIG. 12. shows the docking device 100 being implanted into the mitral valve 16 so that the guard member 104 is on the left atrium side, with the proximal coil region 102p extending into the left atrium 18. As shown, the arms 122 are extended in the deployed orientation so that the panels 140 provide cover over the mitral valve anatomy, which can block PVL.

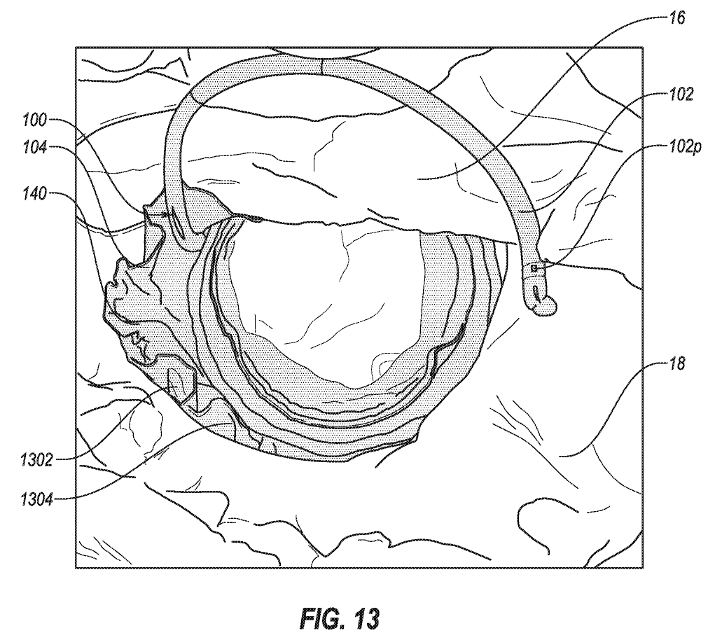

FIG. 13 shows the docking device 100 being implanted into the mitral valve 16 so that the guard member 104 is on the left atrium side, with the proximal coil region 102p extending into the left atrium 18. Compared to FIG. 12, the mitral anatomy in FIG. 13 is smaller. As shown, the arms 122 are extended in the deployed orientation so that the panels 140 are ruffled with protruding portions 1304 and gap portions 1302 at the proximal side. However, it was found that the panels 140 still provided sufficient protection and blockage, so that the guard member can block the PVL. However, FIG. 13 also shows that reducing arm length may be useful for configuring guard members for smaller mitral valve anatomies. This can allow for tailoring of the dimensions to target different age groups or size groups of human patients. In some aspects, the arms may still fully extend, but the guard member may extend up the atrial wall rather than extending straight out from the valve. This design allows deployment the guard member higher above the valve so the arms fully expand and then drop the docking device down onto the leaflets, thereby allowing the guard member to ride up the atrial wall.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking device for securing a prosthetic valve at a native valve, the docking device comprising: a coil comprising a plurality of helical turns when in a deployed orientation; and a guard member attached to the coil by being coupled to at least a portion of a helical turn thereof, wherein the guard member includes a scaffold with a spine and a plurality of arms extending from the spine, wherein the plurality of arms is coupled to a flap, wherein the guard member is movable between a radially compressed state in a delivery orientation and a radially expanded state in the deployed orientation.

Example 2. The docking device of Example 1, wherein when the guard member is in the radially compressed state, the plurality of arms and the flap are radially compressed against the coil in the delivery orientation so that a cross-sectional profile of the docking device includes a diameter that is smaller than a predefined threshold diameter.

Example 3. The docking device of Example 2, wherein the predefined threshold diameter ranges from about 2 mm to about 3 mm.

Example 4. The docking device of any one of Examples 1-3, wherein when the guard member moves from the radially compressed state of the delivery orientation to the radially expanded state of the deployed orientation, the guard member extends radially outwardly relative to the coil by the arms rotating outwardly and extending the flap.

Example 5. The docking device of any one of Examples 1-4, wherein when the guard member is in the radially expanded state in the deployed orientation, the plurality of arms and flap extend radially outward away from the coil and circumferentially along a portion of the coil.

Example 6. The docking device of Example 5, wherein the flap defines an inner edge that is coupled to the coil, wherein the inner edge of the flap has an arc angle that is greater than 180 degrees.

Example 7. The docking device of Example 6, wherein the arc angle ranges from about 240 degrees to about 360 degrees.

Example 8. The docking device of any one of Examples 1-7, wherein the number of arms range from three to eight.

Example 9. The docking device of Example 8, wherein the number of arms ranges from four to six.

Example 10. The docking device of any one of Examples 1-9, wherein the arms of the plurality of arms have substantially the same length.

Example 11. The docking device of any one of Examples 1-9, wherein the guard member includes at least one arm forming at least one lobe by having both ends of the respective at least one arm coupled to the spine.

Example 12. The docking device of Example 11, wherein the at least one lobe includes a terminal lobe a terminal position of the spine.

Example 13. The docking device of Example 12, wherein the terminal position is a trailing position of the spine with curvature of the plurality of arms oriented toward the trailing position.

Example 14. The docking device of any one of Examples 1-13, wherein for each arm, a base portion of the respective arm is attached to the spine and a head portion extends radially outwardly relative to the base portion from the spine when the guard member is in the radially expanded state in the deployed orientation.

Example 15. The docking device of any one of Examples 1-14, wherein each arm extends at an angle relative to the spine when the guard member is in the radially expanded state in the deployed orientation, wherein the angle is less than or about 80 degrees, less than or about 70 degrees, less than or about 60 degrees, less than or about 50 degrees, less than or about 40 degrees, or less than or about 30 degrees.

Example 16. The docking device of Example 15, wherein each arm of the the plurality of arms extends at the angle relative to the spine when the guard member is in the radially expanded state in the deployed orientation, wherein the angle for each arm is within about 10 degrees from each other.

Example 17. The docking device of Example 1, wherein when guard member is in the radially expanded state in the deployed orientation the flap extends from the coil with a radially expanded dimension of about 4 mm to about 30 mm, from about 6 mm to about 25 mm, or about 10.5 mm.

Example 18. The docking device of any one of Examples 1-17, wherein each arm of the plurality of arms has a length from the spine to a tip of about 4 mm to about 30 mm, from about 8 mm to about 25 mm, or about 10.5 mm.

Example 19. The docking device of any one of Examples 1-18, wherein the one or more arms are spaced apart on the spine with about equal distance when the guard member is in the radially expanded state in the deployed orientation, where gaps between the arms define panels of the flap.

Example 20. The docking device of any one of Examples 1-19, wherein the guard member is connected to the coil via one or more sutures.

Example 21. The docking device of any one of Examples 1-20, wherein the guard member is stitched to a cover member of the coil via the one or more sutures.

Example 22. The docking device of any one of Examples 1-21, wherein when the guard member is stitched to a retention member of the coil, wherein the coil includes a coil core, a tubular cover member over the coil core, and the retention member as a tube over the tubular cover member.

Example 23. The docking device of any one of Examples 1-22, wherein each arm is within a sleeve, wherein the sleeve is either coupled with the flap or formed from a sleeve sheet stitched to the flap to form the sleeve.

Example 24. The docking device of any one of Examples 1-22, wherein each arm is loop stitched to the flap.

Example 25. The docking device of any one of Examples 1-23, wherein the flap is stitched to the spine.

Example 26. The docking device of any one of Examples 1-25, wherein when the guard member is in the deployed orientation at the native valve, one or more proximal arms overlay or press against a first portion of a native heart chamber and one or more distal arms overlay or press against a second portion of the native heart chamber that is about opposite to the first portion.

Example 27. The docking device of Example 26, wherein the native valve is a mitral valve, wherein the first portion comprises an anterior leaflet or posterior leaflet of the mitral valve, and the second portion comprises the posterior leaflet of the mitral valve when the first portion comprises the anterior leaflet and comprises the anterior leaflet of the mitral valve when the first portion comprises the posterior leaflet.

Example 28. The docking device of any one of Examples 1-27, wherein when the guard member is in the deployed orientation at a mitral valve, the flap overlays or presses against a posterior leaflet or left atrium region thereof.

Example 29. The docking device of any one of Examples 1-27, wherein when the guard member is in the deployed orientation at a mitral valve, the flap overlays or presses against an anterior leaflet or left atrium region thereof.

Example 30. The docking device of any one of Examples 1-27, wherein when the guard member is in the deployed orientation at a mitral valve, the flap overlays or presses against an anterior leaflet and posterior leaflet or left atrium region thereof.

Example 31. The docking device of any one of Examples 1-30, wherein the scaffold comprises a shape memory material.

Example 32. The docking device of Example 31, wherein the shape memory material comprises nickel titanium alloy.

Example 33. The docking device of any one of Examples 1-32, wherein the flap includes at least one layer of a biocompatible material coupled to the scaffold.

Example 34. The docking device of Example 33, wherein the biological material is flexible so as to be capable of being folded in the delivery orientation and expanded in the deployed orientation.

Example 35. The docking device of any one of Examples 33-34, wherein the biological material is porous and configured for cellular ingrowth.

Example 36. The docking device of any one of Examples 1-35, wherein each arm has a head at a terminal end opposite of the spine, wherein each head includes a rounded shape.

Example 37. The docking device of Example 36, wherein each head includes a teardrop shape with a rounded distal end.

Example 38. The docking device of any one of Examples 1-37, wherein each arm includes a bend so that each arm is either bent clockwise or counter-clockwise.

Example 39. The docking device of Example 38, wherein the bend turns a distal region of the arm to be about parallel with the with the spine, wherein the distal region of each arm has an angle with respect to the spine to be less then or about 10 degrees.

Example 40. The docking device of any one of Examples 36-39, wherein each head of each arm is retained within a sleeve that is coupled with the flap of the guard member.

Example 41. The docking device of any one of Examples 1-40, wherein the flap is formed of at least one sheet of fabric formed by weaving, knitting, crocheting, or bonding fibers together, wherein the fibers are biocompatible. Additional examples include braiding, laminating (e.g., for polymeric coverings), electrospinning (ePTFE, etc), and extrusion (ePTFE), as well as other related methods.

Example 42. The docking device of any one of Examples 1-40, wherein the flap is formed of at least one sheet of material that is polymeric in a form of a membrane, film, plastic sheet, or foil, wherein the sheet material is biocompatible.

Example 43. The docking device of Example 42, wherein the sheet material is expanded polytetrafluoroethylene (ePTFE), polytetrafluoroethylene (PTFE), thermoplastic polyurethane, or silicone.

Example 44. The docking device of any one of Examples 1-43, wherein the flap comprises a peripheral lip configured as an edge proctor coupled to the flap, wherein the edge protector wraps around a distal region of the arms from a top surface to a bottom surface of the flap.

Example 45. The docking device of Example 44, wherein the edge protector is formed of a fabric or a polymeric sheet.

Example 46. The docking device of one of the Examples 1-45, wherein the scaffold includes a uniform thickness that ranges from about 0.10 mm to about 0.5 mm.

Example 47. The docking device of Example 46, wherein each arm has a width that ranges from about 0.10 mm to about 0.30 mm, wherein the width is orthogonal with the thickness.

Example 48. The docking device of one of Examples 46-47, wherein each arm has a length that ranges from about10 mm to about 60 mm.

Example 49. The docking device of one of Examples 1-48, wherein the flap has a dimension from the coil that ranges from about 30 mm to about 70 mm, from about 40 mm to about 60 mm, or about 45 mm to about 55 mm.

Example 50. The docking device of one of Examples 1-49, wherein the flap has a dimension from coil to edge boundary that ranges from about 4 mm to about 30 mm, from about 6 mm to about 20 mm, or about 8 mm to about 10 mm, or about 10.5 mm.

Example 51. The docking device of one of the Examples, wherein an edge protector on a peripheral edge of the guard member is a same material of the flap that is folded over the head of the arms.

Example 52. The docking device of one of the Examples, wherein a base portion of each arm has a width of about 0.1 mm and a region adjacent to the head portion of each arm has a width of about 0.25 mm.

Example 53. The docking device of one of Examples 1-52, wherein the guard member includes a marker band.

Example 54. The docking device of Example 53, wherein the marker band is crimped onto the guard device.

Example 55. The docking device of Example 53, wherein the marker band is crimped onto the guard device and coil.

Example 56. The docking device of one of Examples 1-55, further comprising a covering wrap that is wrapped around the coil and spine, and a marker band crimp at each end of the covering wrap.

Example 57. The docking device of one of Examples 1-56, wherein the spine has an arc in a relaxed state, wherein the arc is at least a portion of a circumference having a diameter, wherein the diameter ranges from about 0.29 mm to about 40 mm, from about 0.31 mm to about 0.38 mm, or about 33 mm to about 35 mm.

Example 58. The docking device of one of Examples 1-57, wherein the coil includes a cover having a first cover region outside of the guard member that is thicker than a second cover region at the guard member.

Example 59. The docking device of one of Examples 1-58, wherein the coil includes a cover having: a first cover region outside of the guard member with a thickness of about 1.6 mm to 2.2 mm or about 1.9 mm; and a second cover region at the guard member with a thickness of about 1.0 mm to about 1.6 mm or about 1.3 mm.

Example 60. The docking device of one of Examples 1-59, wherein the guard member includes a cover substantially enclosing the scaffold, wherein the cover forms the flap.

Example 61. The docking device of Example 60, wherein the cover is formed by two cover sheets coupled together with the scaffold therein.

Example 62. A method for making the docking device of one of Examples 1-61, the method comprising: obtaining the scaffold comprising the spine and a plurality of arms connected to and extending radially outwardly from the spine; coupling the scaffold to the flap to form the guard member; obtaining the coil; and coupling the guard member to the coil.

Example 63. The method of Example 62, further comprising enclosing the scaffold within a cover of the flap to form the guard member of the docking device.

Example 64. The method of one of Examples 62-63, wherein the obtaining the scaffold comprises cutting a substrate to form the spine and the plurality of arms.

Example 65. The method of Example 64, wherein the substrate comprises a nickel-titanium alloy (e.g., Nitinol) sheet, and wherein the cutting comprises laser cutting the Nitinol sheet.

Example 66. The method of any one of Examples 62-65, further comprising stacking two fabric layers together and cutting the two fabric layers using a mold placed over the two fabric layers to create the cover, wherein an outer periphery of the mold defines a rounded shape of the scaffold, and wherein an inner periphery of the mold defines a shape of the spine of the scaffold.

Example 67. The method of Example 66, wherein cutting the two fabric layers comprises moving a heated member (e.g., soldering iron) along the outer periphery of the mold so that the two fabric layers are heat-cut along the outer periphery of the mold and sealed together to form an outer edge of the cover.

Example 68. The method of Example 63, wherein enclosing the scaffold comprises inserting the scaffold between the two fabric layers through an opening that is located radially inwardly of the inner periphery of the mold, wherein the scaffold inserted between the two fabric layers is positioned so that the plurality of arms is aligned with the outer edge of the cover.

Example 69. The method of Example 68, wherein enclosing the wireframe further comprises moving the heated member (e.g., soldering iron) along the inner periphery of the mold so that the two fabric layers are heat-cut along the inner periphery the mold and sealed together to form an inner edge of the cover, wherein the spine of the scaffold extends along the inner edge of the cover.

Example 70. The method of any one of Examples 62-69, further comprising connecting the scaffold to the flap via a plurality of sutures.

Example 71. The method of Example 70, wherein at least some of the sutures extend across one or more arms or one or more spine regions of the scaffold.

Example 72. The method of any one of Examples 70-71, wherein at least some of the sutures stitch a sleeve for each arm to the flap.

Example 73. The method of any one of Examples 62-72, further comprising attaching the guard member to a coil of the docking device by stitching the flap to a coil cover member.

Example 74. The method of any one of Examples 62-73, comprising stitching the guard member to a retention member of the coil, wherein the coil includes a coil core, a tubular cover member over the coil core, and the retention member as a tube over the tubular cover member.

Example 75. The method of Example 72, further comprising forming the sleeve for each arm by cutting a fabric sheet into a shape of the sleeve and stitching the sleeve-shaped fabric sheet in the shape of the sleeve with the flap.

Example 76. The method of Example 72, further comprising forming the sleeve by forming a tube that fits around the respective arm and stitching the tubular sleeve to the flap.

Example 77. The method of one of the Examples 62-76, further comprising forming at least one lobe on the scaffold, wherein the lobe includes a lobe arm connected at both ends to the spine.

Example 78. The method of one of Examples 62-77, further comprising forming a head on each arm of the scaffold.

Example 79. The method of Example 78, wherein forming the head includes cutting the head with the respective arms from a sheet.

Example 80. The method of Example 78, wherein forming the head includes: cutting the arms from a sheet; and bending a distal end of the arm back onto itself to form a loop with a rounded end.

Example 81. The method of one of Examples 62-80, further comprising forming an edge protector on a peripheral lip of the flap.

Example 82. The method of one of Examples 62-81, further comprising forming a marker band onto the guard member.

Example 83. The method of one of Examples 62-81, further comprising forming a marker band onto the guard member and coil.

Example 84. The method of one of Examples 62-83, further comprising: wrapping a cover wrap around a core of the coil and the spine of the scaffold; and crimping or otherwise attaching each end of the cover wrap onto the core and spine.

Example 85. A method of configuring a docking device for delivery to a native valve, the method comprising: providing the docking device of one of the Examples 1-61; compressing the guard member by compressing the arms to fold the flap into the delivery orientation; and inserting the guard member in the delivery orientation into a dock sleeve of a dock delivery system.

Example 86. The method of Example 86, further comprising inserting the coil into the dock sleeve.

Example 87. A method of implanting a docking device into a native valve, the method comprising: providing the docking device of one of Examples 1-61; delivering the docking device to a native valve while the docking device is in a delivery orientation; deploying the coil of the docking device at an annulus of the native valve; and deploying the guard member into the deployed orientation at a position at the native valve so that the guard member overlays or presses against the native valve and/or native heart chamber associated with the native valve.

Example 88. The method of Example 87, further comprising deploying the guard member results in rotation of the plurality of arms and radial expansion of the flap.

Example 89. The method of one of Examples 87-88, wherein when the guard member is in the deployed orientation at the native valve, one or more proximal arms overlay or press against a first portion of a native heart chamber and one or more distal arms overlay or press against a second portion of the native heart chamber that is about opposite to the first portion.

Example 90. The method of Example 89, wherein the native valve is a mitral valve, wherein the first portion comprises an anterior leaflet or posterior leaflet of the mitral valve, and the second portion comprises the posterior leaflet of the mitral valve when the first portion comprises the anterior leaflet and comprises the anterior leaflet of the mitral valve when the first portion comprises the posterior leaflet.

Example 91. The method of one of Example 87-90, wherein when the guard member is in the deployed orientation at a mitral valve, the flap overlays or presses against a posterior leaflet or left atrium region thereof.

Example 92. The method of one of Example 87-91, wherein when the guard member is in the deployed orientation at a mitral valve, the flap overlays or presses against an anterior leaflet or left atrium region thereof.

Example 93. The method of one of Example 87-92, wherein when the guard member is in the deployed orientation at a mitral valve, the flap overlays or presses against an anterior leaflet and posterior leaflet or left atrium region thereof.

Example 94. The method of one of Examples 87-93, wherein the coil remains in a substantially straight configuration in the delivery orientation when delivering the docking device and the coil member moves to a helical configuration after the docking device is deployed.

Example 95. The method of one of Examples 87-94, wherein the guard member remains in the delivery orientation when delivering the docking device and moves to the deployed orientation after the docking device is deployed.

Example 96. The method of one of Example 87-95, wherein delivering the docking device comprises retaining the docking device within a dock sleeve, wherein deploying the docking device comprises moving the docking device out of the dock sleeve.

Example 97. The method of any one of Examples 87-96, wherein deploying the docking device comprises removing a delivery sleeve from the coil and guard member while at the native valve.

Example 98. A method if implanting a prosthetic valve, comprising: providing the docking device of one of Example 1-61; delivering the docking device to a native valve; deploying the docking device at an annulus of the native valve so that the guard member expands into the deployed orientation at a position at the native valve so that the guard member overlays or presses against the native valve and/or native heart chamber associated with the native valve; and deploying a prosthetic valve within the docking device, wherein the coil remains in a substantially straight delivery orientation when delivering the docking device and moves to a helical configuration after the docking device is in the deployed orientation, wherein the guard member remains in a folded delivery orientation when delivering the docking device and moves to an unfolded deployed orientation after the docking device is deployed.

Example 99. A coil for a docking device for securing a prosthetic valve, the coil comprising: a longitudinal axis extending through a lumen of the coil from an inflow side to an outflow side; a first coil region defining a first lumen diameter and configured to be disposed on the inflow side of a native annulus and to stabilize the coil relative to the native annulus; and a second coil region extending from a distal end of the first coil region and comprising one or more helical turns each defining a second lumen diameter and configured to be disposed on an outflow side of a native annulus and to receive a prosthetic valve, wherein a proximal end portion of the first coil region is lifted relative to a plane defined by the first coil region and normal to the longitudinal axis and wherein a proximal end of the first coil region is above the plane defined by the first coil region by less than 12 mm.

Example 100. The coil of any example herein, particularly example 99, further comprising a leading coil extending from the distal end of the second coil region and extending radially outward from the second diameter.

Example 101. The coil of any example herein, particularly any one of examples 99-100, wherein the proximal end portion of the first coil region is lifted at an angle relative to a plane defined by the first coil region and normal to the longitudinal axis.

Example 102. The coil of any example herein, particularly example 101, wherein the angle is within a range of 10 to 50 degrees.

Example 103. The coil of any example herein, particularly any one of examples 99-102, further comprising an attachment portion comprising one or more eyeholes disposed at the proximal end portion of the first coil region.

Example 104. The coil of any example herein, particularly any one of examples 99-103, wherein the first lumen diameter and the second lumen diameter are substantially equal.

Example 105. The coil of any example herein, particularly any one of examples 99-103, wherein the first lumen diameter is in a range of 10 percent to 30 percent greater than the second lumen diameter.

Example 106. The coil of any example herein, particularly one of examples 99-103, wherein the first lumen diameter is in a range of 25 mm to 30 mm and the second lumen diameter is in a range of 20 mm to 25 mm.

Example 107. The coil of any example herein, particularly any one of examples 99-106, wherein the first coil region comprises a single stabilization turn.

Example 108. A docking device comprising the coil of any example herein, particularly any one of examples 99-107, and further comprising a guard member coupled at least partially to an outflow side of the stabilization turn wherein the guard member is movable between a radially compressed state and a radially expanded state.

Example 109. The docking device of any example herein, particularly example 108, wherein the guard member comprises a scaffold and the scaffold comprises a spine, a plurality of arms, and one or more terminal lobes.

Example 110. The docking device of any example herein, particularly any one of examples 108-109, wherein the spine of the scaffolding further comprises a kickout portion configured to wrap around the stabilization turn.

Example 111. The docking device of any example herein, particularly example 110, wherein the guard member comprises a lateral terminal lobe and a medial terminal lobe, and wherein the kickout portion is located proximal to the medial terminal lobe.

Example 112. The docking device of any example herein, particularly any one of examples 109-111, wherein the scaffold further comprises one or more retention elements.

Example 113. The docking device of any example herein, particularly example 112, wherein the retention elements comprise tines, wherein the tines are attached to the arms at a base portion and wherein the tines taper to a point at a tip portion.

Example 114. A coil for a docking device for securing a prosthetic valve, the coil comprising: a longitudinal axis extending through a lumen of the coil from an inflow side to an outflow side; a first coil region configured to be disposed on an inflow side of a native annulus and to stabilize the coil relative to the native annulus; and a second coil region extending from a distal end of the first coil region and comprising one or more helical turns configured to be disposed on an outflow side of a native annulus and to receive a prosthetic valve wherein the coil omits a raised stabilization portion.

Example 115. The coil of any example herein, particularly example 114, further comprising a leading coil extending from the distal end of the second coil region and extending radially outward from a diameter of the second coil region.

Example 116. The coil of any example herein, particularly any one of examples 114-115, wherein a proximal end portion of the first coil region is lifted at an angle relative to a plane defined by the first coil region and normal to the longitudinal axis.

Example 117. The coil of any example herein, particularly example 116, wherein the angle is within a range of 10 to 30 degrees.

Example 118. The coil of any example herein, particularly any one of examples 116-117, wherein the proximal end of the first coil region is above the plane defined by the first coil region by less than 12 mm.

Example 119. The coil of any example herein, particularly any one of examples 114-118, wherein the first coil region defines a first lumen diameter and the second coil region defines a second lumen diameter.

Example 120. The coil of any example herein particularly example 119, wherein the first lumen diameter and the second lumen diameter are substantially equal.

Example 121. The coil of any example herein, particularly example 119, wherein the first lumen diameter is in a range of 10 percent to 30 percent greater than the second lumen diameter.

Example 122. The coil of any example herein, particularly example 119, wherein the first lumen diameter is in a range of 25 mm to 30 mm and the second lumen diameter is in a range of 20 mm to 25 mm.

Example 123. A docking device comprising the coil of any example herein, particularly any one of examples 114-122, and further comprising a guard member coupled at least partially to an outflow side of the stabilization coil wherein the guard member is movable between a radially compressed state and a radially expanded state

Example 124. The docking device of any example herein, particularly example 123, wherein the guard member comprises a scaffold and the scaffold comprises a spine, a plurality of arms, and one or more terminal lobes.

Example 125. The docking device of any example herein, particularly any one of examples 123-124, wherein the guard member is coupled to the outflow side of the stabilization coil and extends circumferentially between 180 and 330 degrees on the outflow side of the stabilization turn.

Example 126. The docking device of any example herein, particularly any one of examples 124-125, wherein the spine of the scaffolding further comprises a kickout portion configured to wrap around the stabilization turn.

Example 127. The docking device of any example herein, particularly any one of examples 124-126, wherein a portion of the guard member wraps around the stabilization turn and extends circumferentially between 30 and 135 degrees on an inflow side of the stabilization turn.

Example 128. The docking device of any example herein, particularly any one of examples 124-127, wherein the scaffold further comprises one or more retention elements.

Example 129. The docking device of any example herein, particularly example 128, wherein the retention elements comprise tines, wherein the tines are coupled to the arms at a base portion and wherein the tines taper to a point at a tip portion.

Example 130. A docking device for securing a prosthetic implant at a native valve, the docking device comprising: a coil defining a longitudinal axis extending through a lumen of the coil from an inflow side to an outflow side and comprising a plurality of helical turns when deployed at the native valve, wherein at least one of the helical turns comprises a first coil region configured to be disposed on the inflow side of a native annulus and to stabilize the coil relative to the native annulus wherein a proximal end portion of the first coil region is lifted relative to a plane defined by the first coil region and normal to the longitudinal axis and wherein a proximal end of the first coil region is above the plane defined by the first coil region by less than 12 mm and at least one of the helical turns comprises a second coil region extending from a distal end of the first coil region and configured to be disposed on the outflow side of a native annulus and to receive a prosthetic valve; and a guard member coupled at least partially to the outflow side of the first coil region wherein the guard member is movable between a radially compressed state and a radially expanded state.

Example 131. The docking device of any example herein, particularly example 130, wherein a portion of the guard member is coupled at least 225 degrees around a circumference of the first coil region.

Example 132. The docking device of any example herein, particularly example 130, wherein a portion of the guard member is coupled at least 270 degrees around a circumference of the first coil region.

Example 133. The docking device of any example herein, particularly example 130, wherein a portion of the guard member is coupled to the outflow side of the first coil region at least 270 degrees around a circumference of the first coil region and a portion of the guard member is disposed on the inflow side of the first coil region at least 15 degrees around a circumference of the of the first coil region.

Example 134. The docking device of any example herein, particularly any one of examples 130-133, wherein the guard member comprises a scaffold and the scaffold comprises a spine, a plurality of arms, and one or more terminal lobes.

Example 135. The docking device of any example herein particularly example 134, wherein the spine of the scaffold further comprises a kickout portion configured to wrap around the stabilization turn.

Example 136. The docking device of any example herein, particularly any one of examples 134-135, wherein the scaffold further comprises one or more retention elements.

Example 137. The docking device of any example herein, particularly example 136, wherein the retention elements comprise tines, wherein the tines are coupled to the arms at a base portion and wherein the tines taper to a point at a tip portion.

Example 138. A method comprising: delivering the docking device of any example herein, particularly any one of examples 108-113 and or 130-137 to a native valve; deploying the docking device at an annulus of the native valve; and deploying a prosthetic valve within the docking device, wherein the coil remains in a substantially straight configuration when delivering the docking device and moves to a helical configuration after the docking device is deployed.

Example 139. A coil for a docking device for securing a prosthetic valve, the coil comprising: a core comprising a plurality of helical turns and defining a longitudinal axis extending through a lumen of the plurality of helical turns from an inflow side to an outflow side when deployed at a native valve, wherein at least one of the helical turns comprises a first region configured to be disposed on the inflow side of a native annulus and to stabilize the coil relative to the native annulus and at least one of the helical turns comprises a second region extending from a distal end of the first region and configured to be disposed on the outflow side of a native annulus and to receive a prosthetic valve; and a cover encompassing at least a portion of the core and comprising a first outer diameter and a second outer diameter which is larger than the first outer diameter, wherein the second region comprises the second outer dim.

Example 140. The coil of any example herein, particularly example 138, wherein at least a portion of the first region is covered by a cover of the second diameter.

Example 141. The coil of any example herein, particularly any one of examples 139-140, wherein the cover comprises a first cover with a first outer diameter and as second cover with a second outer diameter, wherein the first cover and the second cover are two separate pieces.

Example 142. The coil of any example herein, particularly example 141, further comprising a transition region, in which the first cover is flared radially outward such that it overlaps with the second cover in an axial direction.

Example 143. The coil of any example herein, particularly example 142, further comprising a coupling member wrapped around the first cover.

Example 144. The coil of any example herein, particularly example 143, wherein the coupling member comprises suture.

Example 145. A guard member, for a docking device for securing a prosthetic implant at a native valve, the guard member comprising a scaffold with a spine and a plurality of arms extending from the spine; and one or more retention elements coupled to one of the arms of the plurality of arms; wherein the guard member is configured to be attached to a coil by being coupled to at least a portion of a helical turn thereof, wherein the guard member is movable between a radially compressed state in a delivery orientation and a radially expanded state in a deployed orientation.

Example 146. The guard member of any example herein, particularly example 145, further comprising a flap wherein the plurality of arms are coupled to the flap and at least a portion of the scaffold is encompassed by the flap, and wherein the one or more retention elements extend though the flap.

Example 147. The guard member of any example herein, particularly any one of examples 145-146, wherein the retention elements are tines configured to engage heart tissue to help ensure device stability.

Example 148. The guard member of any example herein, particularly example 147, wherein the tines comprise a base portion which is coupled to the arm and has a first width, and a tip portion comprising a second width, wherein the second width is smaller than the first width.

Example 149. The guard member of any example herein, particularly any one of examples 147-148, wherein the tines comprise a tip portion that is a point.

Example 150. The guard member of any one of any example herein, particularly any one of examples 145-149, further comprising one or more terminal lobes.

Example 151. The guard member of any example herein, particularly any one of examples 145-150, wherein when the guard member is in the radially expanded state in the deployed orientation, the plurality of arms and flap extend radially outward away from the coil and circumferentially along a portion of the coil.

Example 152. The guard member of any example herein, particularly any one of examples 145-151, wherein when the guard member is in the radially expanded state, the retention elements extend from the arms in a clockwise direction.

Example 153. The guard member of any example herein, particularly any one of examples 147-152, wherein the tines extend out of a plane defined by the scaffold.

Example 154. The guard member of any example herein, particularly example 153, wherein an angle in a range of 0 to 90 degrees is formed between the tines and the plane defined by the scaffold.

Example 155. The guard member of any example herein particularly example 153, wherein an angle in a range of 0 to 45 degrees is formed between the tines and the plane defined by the scaffold.

Example 156. The guard member of any example herein, particularly example 153, wherein an angle in a range of 90 to 180 degrees is formed between the tines and the plane defined by the scaffold.

Example 157. A method comprising sterilizing the docking device, coil, or guard member of any example herein, particularly any one of examples 1-61 or examples 99-156.

Example 158. A method of treating a heart on a simulation, the method comprising: deploying a docking device at a target location; and deploying a prosthetic valve within the docking device; wherein the docking device is according to any one of examples 1-61 or examples 99-156.

The features described herein with regard to any example can be combined with other features described in any one or more of the other examples, unless otherwise stated. For example, any one or more of the features of one docking device can be combined with any one or more features of another docking device. As another example, any one or more features of one guard member can be combined with any one or more features of another guard member.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A docking device for securing a prosthetic valve at a native valve, the docking device comprising:
a coil comprising a plurality of helical turns when in a deployed orientation; and
a guard member attached to the coil by being coupled to at least a portion of a helical turn thereof, wherein the guard member includes a scaffold with a spine and a plurality of arms extending from the spine, wherein the plurality of arms is coupled to a flap, wherein the guard member is movable between a radially compressed state in a delivery orientation and a radially expanded state in the deployed orientation.

2. The docking device of claim 1, wherein when the guard member is in the radially compressed state, the plurality of arms and the flap are radially compressed against the coil in the delivery orientation so that a cross-sectional profile of the docking device includes a diameter that is smaller than a predefined threshold diameter.

3. The docking device of claim 2, wherein the predefined threshold diameter ranges from about 2 mm to about 3 mm.

4. The docking device of claim 1, wherein when the guard member moves from the radially compressed state of the delivery orientation to the radially expanded state of the deployed orientation, the guard member extends radially outwardly relative to the coil by the arms rotating outwardly and extending the flap.

5. The docking device of claim 1, wherein the plurality of arms is in a range from three to eight arms.

6. The docking device of claim 1, wherein the guard member is stitched to a cover member of the coil via one or more sutures.

7. A coil for a docking device for securing a prosthetic valve, the coil comprising:
a longitudinal axis extending through a lumen of the coil from an inflow side to an outflow side;
a first coil region defining a first lumen diameter and configured to be disposed on the inflow side of a native annulus and to stabilize the coil relative to the native annulus; and
a second coil region extending from a distal end of the first coil region and comprising one or more helical turns each defining a second lumen diameter and configured to be disposed on an outflow side of a native annulus and to receive a prosthetic valve,
wherein a proximal end portion of the first coil region is lifted relative to a plane defined by the first coil region and normal to the longitudinal axis and wherein a proximal end of the first coil region is above the plane defined by the first coil region by less than 12 mm.

8. The coil of claim 7, further comprising a leading coil extending from the distal end of the second coil region and extending radially outward from the second diameter.

9. The coil of claim 7, wherein the proximal end portion of the first coil region is lifted at an angle relative to a plane defined by the first coil region and normal to the longitudinal axis.

10. The coil of claim 9, wherein the angle is within a range of 10 to 50 degrees.

11. The coil of claim 7, further comprising an attachment portion comprising one or more eyeholes disposed at the proximal end portion of the first coil region.

12. The coil of claim 7, wherein the first lumen diameter is in a range of 25 mm to 30 mm and the second lumen diameter is in a range of 20 mm to 25 mm.

13. A docking device for securing a prosthetic implant at a native valve, the docking device comprising:
a coil defining a longitudinal axis extending through a lumen of the coil from an inflow side to an outflow side and comprising a plurality of helical turns when deployed at the native valve, wherein at least one of the helical turns comprises a first coil region configured to be disposed on the inflow side of a native annulus and to stabilize the coil relative to the native annulus wherein a proximal end portion of the first coil region is lifted relative to a plane defined by the first coil region and normal to the longitudinal axis and wherein a proximal end of the first coil region is above the plane defined by the first coil region by less than 12 mm and at least one of the helical turns comprises a second coil region extending from a distal end of the first coil region and configured to be disposed on the outflow side of a native annulus and to receive a prosthetic valve; and
a guard member coupled at least partially to the outflow side of the first coil region wherein the guard member is movable between a radially compressed state and a radially expanded state.

14. The docking device of claim 13, wherein a portion of the guard member is coupled at least 225 degrees around a circumference of the first coil region.

15. The docking device of claim 13, wherein a portion of the guard member is coupled at least 270 degrees around a circumference of the first coil region.

16. The docking device of claim 13, wherein a portion of the guard member is coupled to the outflow side of the first coil region at least 270 degrees around a circumference of the first coil region and a portion of the guard member is disposed on the inflow side of the first coil region at least 15 degrees around a circumference of the of the first coil region.

17. A coil for a docking device for securing a prosthetic valve, the coil comprising:
a core comprising a plurality of helical turns and defining a longitudinal axis extending through a lumen of the plurality of helical turns from an inflow side to an outflow side when deployed at a native valve, wherein at least one of the helical turns comprises a first region configured to be disposed on the inflow side of a native annulus and to stabilize the coil relative to the native annulus and at least one of the helical turns comprises a second region extending from a distal end of the first region and configured to be disposed on the outflow side of a native annulus and to receive a prosthetic valve; and
a cover encompassing at least a portion of the core and comprising a first outer diameter and a second outer diameter which is larger than the first outer diameter, wherein the second region comprises the second outer dim.

18. The coil of claim 17, wherein at least a portion of the first region is covered by a cover of the second diameter.

19. The coil of claim 18, wherein the cover comprises a first cover with a first outer diameter and as second cover with a second outer diameter, wherein the first cover and the second cover are two separate pieces.

20. The coil of claim 19, further comprising a transition region, in which the first cover is flared radially outward such that it overlaps with the second cover in an axial direction.
